(19) United States
(12) Patent Application Publication
(10) Pub. No.: US 2024/0384269 A1
(43) Pub. Date: Nov. 21, 2024

(54) COMPOSITIONS CONTAINING NUCLEIC ACID NANOPARTICLES AND PROCESSES RELATED TO ALTERATION OF THEIR PHYSIOCHEMICAL CHARACTERISTICS

(71) Applicant: Sixfold Bioscience Ltd., London (GB)

(72) Inventors: George William Foot, Dorchester (GB); Anna Perdrix Rosell, London (GB); James Luke Rushworth, London (GB)

(21) Appl. No.: 18/555,456

(22) PCT Filed: Apr. 15, 2022
(86) PCT No.: PCT/IB2022/000218

§ 371 Date: Oct. 13, 2023

Related U.S. Application Data
(60) Provisional application No. 63/175,185, filed on Apr. 15, 2021.

(51) Int. Cl. C12N 15/11

(52) U.S. Cl. CPC C12N 15/111; C12N 2310/314; C12N 2310/3341; C12N 2310/3513; C12N 2310/3519; C12N 2310/51; C12N 2320/32; C12N 2320/50; C12N 2330/30

(57) ABSTRACT

The invention provides therapeutic compositions that include nucleic acid nanoparticles for delivery of cargo and methods of using the same.

### FIELD OF THE INVENTION

The invention relates generally to therapeutic compositions that include nucleic acid nanoparticles for delivery of cargo and methods of using the same.

### BACKGROUND

Nucleic acids (NAs) nanotechnology offers an exciting opportunity to assemble structures with precise control of their properties at the nanoscale. By harnessing the base-pairing interactions, the size, shape and placement of cargo molecules of the self-assembled construct can be fully controlled. Interestingly, the resulting structures have different properties compared to their linear components. For example, they can resist enzymatic degradation or can be retained in tissues longer. Some structures have even been shown to enter cells without the need for transfection agents. Therefore, NA constructs have gained interest for various biological applications, including imaging, sensing, and delivery. They offer a particularly interesting platform to improve NA therapeutics delivery. Indeed, while NA therapeutics, made of linear oligonucleotides, have a huge potential to address unmet pharmaceutical needs, they still suffer from challenges that need to be urgently addressed: their delivery is restricted to the liver, they have poor cellular uptake and are retained in the endosome.

By integrating NAs therapeutics within the programmable assemblies, the PK/PD properties are expected to be altered, resulting in new distribution and cellular uptake profiles.

Indeed, the shape, size, charge, and hydrophobicity are known parameters that affect tissue distribution. While the shape and size can be tuned by changing structural design, the other parameters can be changed by integrating chemical modifications. Oligonucleotides are synthesized with precise sequence control and offer a platform to introduce selected chemical modifications at the desired stoichiometry. For example, 2′F-modifications are introduced in RNA strands to increase nuclease resistance. One of the main goals of affecting PK/PD properties is to achieve precise tissue and cell-targeting-a particularly urgent need in the context of cancer treatments, where current therapies are efficient, but cause too many off-target side effects.

### SUMMARY

In this invention, we describe how to introduce chemical modifications within the composition in an efficient manner, to systematically screen the resulting effects of both the position and the nature of the modification. Ultimately, this will result in favorable PK/PD properties and improve therapeutics delivery. This invention is particularly exciting in the context of tumor delivery of NA therapeutics, but not limited to, and can be applied to the delivery of all types of therapeutics, as the composition can be conjugated with small molecules, peptides, antibodies and any relevant therapies.

Use of phosphoramidite chemistry to alter the physicochemical characteristics of nucleic acid nanoparticles Generation of RNA strands and functionalization post-synthetically is ideal for a research and development setting, however there may be issues with scale-up, particularly for some bioconjugation reactions. To overcome these potential pitfalls, it would be extremely useful to develop a new type of phosphoramidite to which any modification could be attached. These should be subdivided into two distinct categories: universal internal modifications and polymeric tails that can drastically alter both the pharmacokinetics (PK) and pharmacodynamics (PD) of the nucleic acid nanoparticles (for example FIG. 7).

The universal internal modification will be inosine modified with an appropriate reactive click handle. Inosine is considered to be a “universal base”, as it can form hydrogen bonds with all four canonical bases (G. Butora, D. M. Kenski, A. J. Cooper, W. Fu, N. Qi, J. J. Li, W. M. Flanagan, I. W. Davies, Nucleoside Optimization for RNAi: A High-Throughput Platform, J. Am. Chem. Soc. 133 (2011) 16766-16769). From a synthetic point of view, gram-scale synthesis of this universal base would be simpler than modifying all four bases (A, G, C, U). Alternatively, to inosine, any of the following moieties might be used as a universal base: 2′-deoxynebularine. 3-nitropyrrole 2′-deoxynucleoside, 5′-nitroindole 2′-deoxynucleoside (D. Loakes, D. M. Brown, 5-Nitroindole As an Universal Base Analogue, Nucleic Acids Res. 22 (1994) 4039-4043), 6H, 8H-3,4-dihydro-pyrimido[4,5-c][1,2] oxazin-7-one (P) and 2-amino-9-(2-deoxy-β-ribofuranosyl)-6-methoxyaminopurine.

The aforementioned modifications could be used for coupling in solution. However, a solid phase synthesis approach is also possible. Solid phase oligonucleotide synthesis allows one to introduce a wide variety of different chemically modified moieties into the sequence utilizing phosphoramidite building blocks. The presence of a 4,4′-dimethoxytrityl (DMT) group at the modification of interest allows further elongation of a growing oligonucleotide resulting in formation of an oligonucleotide that can contain different types of customized moieties, such as spacers, linkers or molecules that alter the overall physicochemical properties (K. Bartosik, K. Debiec, A. Czarnecka, E. Sochacka, G. Leszczynska, Synthesis of nucleobase-modified RNA oligonucleotides by post-synthetic approach, Molecules. 25 (2020)).

One very interesting exploitation of such modifications in the nanoparticle field is an approach to generate a desired conjugate straight during the solid phase oligonucleotide synthesis (T. Yamamoto, C. Terada, K. Kashiwada, A. Yamayoshi, M. Harada-Shiba, S. Obika, Synthesis of Monovalent N-Acetylgalactosamine Phosphoramidite for Liver-Targeting Oligonucleotides, Curr. Protoc. Nucleic Acid Chem. 78 (2019) e99); (I. Cedillo, D. Chreng, E. Engle, L. Chen, A. K. McPherson, A. A. Rodriguez, Synthesis of 5-GalNAc-conjugated oligonucleotides: A comparison of solid and solution-phase conjugation strategies, Molecules. 22 (2017) 1-12). In contrast to post-synthetic conjugation in solution, this approach allows one to potentially reduce the number of impurities, time and resources and can significantly improve the overall yield of a conjugation.

Herein, we present a successful incorporation of a disulfide linkage into the growing oligonucleotide to increase an overall yield of siRNA-oligonucleotide conjugation when compared to standard approach in solution.

As an extension to the solid phase approach, a potentially straightforward way of altering PK/PD without having too much of an effect on assembly would be to develop a simplified “modifier”-type molecule that can be extended to form oligophosphates. Synthesis of these types of molecules has been explored in the literature (D. de Rochambeau, Y. Sun, M. Barlog, H. S. Bazzi, H. F. Sleiman, Modular Strategy To Expand the Chemical Diversity of DNA and Sequence-Controlled Polymers, J. Org. Chem. 83 (2018) 9774-9786) and is known in the art (US 2019/0060324), however, using them for specific modulation of PK/PD properties has not been widely explored. The flexibility and control of solid phase phosphoramidite synthesis will also allow for a high degree of fine-tuning and will be possible at a high-throughput level.

In addition to these novel PK/PD altering phosphoramidites, the proposed invention will also utilise bioconjugation strategies using known and novel compounds, which are described herein (M. L. W. J. Smeenk, J. Agramunt, K. M. Bonger, Recent developments in bioorthogonal chemistry and the orthogonality within, Curr. Opin. Chem. Biol. 60 (2021) 79-88); ([1] B. L. Oliveira, Z. Guo, G. J. L. Bernardes, Inverse electron demand Diels-Alder reactions in chemical biology, Chem. Soc. Rev. 46 (2017) 4895-4950). Functionalization can be performed according to proposed strategies either on pre-assembled constructs or directly to the core strands followed by assembly. The reactive moiety can be introduced more than once to the 5′ end of the core strand or as various combinations of reactive moieties following the principles of orthogonal labelling.

Alterations to Nucleic Acid Nanoparticles to Alter their Physicochemical Properties

### 1. Cargo

Accessing the right organ is one of the most important steps for any delivery agent. The organs can be targeted by modulating the charge, size, and the shape of the agent, along with the protein corona. Peptides are naturally occurring biopolymers that possess different amino acid side chains with varying desirable properties for charge, size, and nature. Depending on the chosen amino acids, peptides can be classified as positively charged, negatively charged or neutral and hydrophilic or hydrophobic peptides. Such a diverse set of peptides can contribute to alter PK/PD properties of cargo (see for example FIG. 7), behave as a targeting moiety of a particular cell type in an organ and/or aid in endosomal escape (see for example Table 1 and Table 5). In addition, the incorporation of peptides can target a particular type of protein that can change the type of protein corona being formed which can then facilitate differential organ targeting. The peptides can be conjugated internally or at the terminus. The peptides can be conjugated using NHS-Maleimide-Thiol reactions, Copper catalyzed click reactions, Inverse electron demand Diels-Alder reactions, strain promoted alkyne-azide cycloaddition (Table 2) or enzymatic conjugation methods (Table 3).

### 2. Changing the Nucleic Acid Backbone for Enhanced Stability Against Nuclease Degradation.

L-RNA is the left-turning and mirror image version of natural RNA, as opposed to the naturally occurring right-turning version called D-RNA (FIG. 11). L-RNA is important for two reasons: (i) its stability over nuclease degradation and (ii) its ability to assemble and produce nanoconstructs. L-RNA is significantly more stable than D-RNA against degradation by various commercial phosphodiesterases or by nucleases in human serum (M. J. Damha, P. A. Giannaris, P. Marfey, Antisense L/D-Oligodeoxynucleotide Chimeras: Nuclease Stability, Base-Pairing Properties, and Activity at Directing Ribonuclease H, Biochemistry. 33 (1994) 7877-7885); (S. Klulβmann, A. Nolte, R. Bald, V. A. Erdmann, J. P. Fürste, Mirror-image RNA that binds D-Adenosine, Nat. Biotechnol. 14 (1996) 1112-1115). This alleviates the need for further modifications on the nanoconstruct. Since the two enantiomers are identical in structure other than their chiral differences, their intrinsic physical properties are generally equal to each other. This includes duplex stability, solubility, and selectivity as D-RNA, but form a left-helical double-helix. Because of the helicity, the construct should be made fully L-RNA or D-RNA (for successful assemblies, as each addition will reduce Tm). The constructs can be constructed using the routine solid phase manufacturing (phosphoramidite commercially available). Compared to other modifications such as 2′F or 2′OMe, L-RNA bases show reduced protein binding profiles when incorporated into 3D structure (H. B. D. Thai, K. R. Kim, K. T. Hong, T. Voitsitskyi, J. S. Lee, C. Mao, D. R. Ahn, Kidney-Targeted Cytosolic Delivery of siRNA Using a Small-Sized Mirror DNA Tetrahedron for Enhanced Potency, ACS Cent. Sci. 6 (2020) 2250-2258). L-RNA nanoconstructs exhibit an increased circulation time and result in extrahepatic tissue accumulation.

### 3. Increased Loading Capacity

Many different chemical strategies are currently used to successfully deliver a molecule of interest into a biological target. As a result of bioconjugation strategies (see for example Table 2 and Table 3), upon which a new covalent bond is formed, many targeting units, i.e., peptides, can be attached to a therapeutic molecule in order to increase internalization into tumor cells. The nature of a chemical bond allows scientists to predict not only the design of the most efficient pathway for successful delivery but also to control the mechanism of release of the therapeutic moiety. (E. I. Vrettos, T. Karampelas, N. Sayyad, A. Kougioumtzi, N. Syed, T. Crook, C. Murphy, C. Tamvakopoulos, A. G. Tzakos, Development of programmable gemcitabine-GnRH pro-drugs bearing linker controllable “click” oxime bond tethers and preclinical evaluation against prostate cancer, Eur. J. Med. Chem. 211 (2021) 113018); (M. Dirin, E. Urban, B. Lachmann, C. R. Noe, J. Winkler, Concise postsynthetic preparation of oligonucleotide-oligopeptide conjugates through facile disulfide bond formation, Future Med. Chem. 7 (2015) 1657-1673).

Increasing the complexity of the delivery system by adding more units, especially if more than one chemical approach has been utilized, increases the complexity of the synthesis, and can have a counterproductive effect on biological activity. To overcome any potential issues, the applied synthetic strategy should be designed and executed in a way that the newly formed molecule is compatible with all subsequent modifications and therefore the previously introduced bonds remain intact throughout the whole manufacturing process in addition to surviving metabolic pathways further downstream.

The main goal of that application is to improve the loading capacity, targeting and controlled release of each component as a result of attaching a therapeutic together with targeting moiety into the drug delivery system. This can be achieved by implementing effective design changes to the nucleic acid nanoparticle.

The efficiency of a drug delivery vehicle can be estimated with how much drug reaches the target. Minimum dosage to reach therapeutic activity is the target. Increasing the number of therapeutic cargoes loaded onto the drug delivery systems, e.g., prepare multivalent systems, is one way to achieve this. Multivalency aims at delivering multiple drugs at once for synergistic effects and/or higher therapeutic index. Multivalent strategies have been shown to increase efficacy, e.g., divalent siRNAs where effects can be seen up to 6 months post-treatment (J. F. Alterman, B. M. D. C. Godinho, M. R. Hassler, C. M. Ferguson, D. Echeverria, E. Sapp, R. A. Haraszti, A. H. Coles, F. Conroy, R. Miller, L. Roux, P. Yan, E. G., Knox, A. A. Turanov, R. M. King, G. Gernoux, C. Mueller, H. L. Gray-Edwards, R. P. Moser, N. C. Bishop, S. M. Jaber, M. J. Gounis, M. Sena-Esteves, A. A. Pai, M. DiFiglia, N. Aronin, A. Khvorova, A divalent siRNA chemical scaffold for potent and sustained modulation of gene expression throughout the central nervous system, Nat. Biotechnol. 37 (2019) 884-894). Yet, common synthetic approaches span from two extremes: either two to three molecules can be attached, or hundreds (G. Yamankurt, R. J. Stawicki, D. M. Posadas, J. Q. Nguyen, R. W. Carthew, C. A. Mirkin, The effector mechanism of siRNA spherical nucleic acids, Proc. Natl. Acad. Sci. U.S.A 117 (2020) 1312-1320). Multivalent systems carrying therapeutic moieties with perfect control over their identity and numbers are still lacking (for example, the ability to attach three different drugs in different ratios). Currently attachment of functional RNAs to nucleic acid-based delivery vehicles is achieved by strand hybridization or using various bioconjugation chemistries such as CuAAC, IEDDA, SPAAC, etc. (Q. Hu, S. Wang, L. Wang, H. Gu, C. Fan, DNA Nanostructure-Based Systems for Intelligent Delivery of Therapeutic Oligonucleotides, Adv. Healthc. Mater. 7 (2018) 1-19); (H. Zhang, G. S. Demirer, H. Zhang, T. Ye, N. S. Goh, A. J. Aditham, F. J. Cunningham, C. Fan, M. P. Landry, DNA nanostructures coordinate gene silencing in mature plants, Proc. Natl. Acad. Sci. U.S.A 116 (2019) 7543-7548); (H. Xue, F. Ding, J. Zhang, Y. Guo, X. Gao, J. Feng, X. Zhu, C. Zhang, DNA tetrahedron-based nanogels for siRNA delivery and gene silencing, Chem. Commun. 55 (2019) 4222-4225); (K. Astakhova, R. Ray, M. Taskova, J. Uhd, A. Carstens, K. Morris, “Clicking” Gene Therapeutics: A Successful Union of Chemistry and Biomedicine for New Solutions, Mol. Pharm. 15 (2018) 2892-2899).

The proposed approach will simplify how to increase loading capacity, while allowing to retain full control over the stoichiometry. Additionally, the approach makes processes more environmentally friendly and sustainable by minimizing the amounts of material required for delivery vehicles, cargo to be loaded (such as functional RNAs) as well as reagents needed.

The invention shows a novel use of incorporation of branching phosphoramidites in adding more functional NAs such as siRNA, mRNA, miRNA, shRNA, InRNA, antisense oligonucleotides, aptamers etc. to the composition. Branching units will be introduced in one or more of the nucleic acid nanoparticle component strands (for example FIG. 8-10). Cargoes are not limited to nucleic acids if they are compatible with phosphoramidite chemistry/or any bioconjugation chemistries (see for example Table 5). Loading capacity can be expanded from 2 to exponential numbers depending on the number of branching units introduced.

### Methods for the Compaction of Nucleic Acids Using Origami

Scaffolded origami offers a programmable nanoscale platform for the controlled self-folding of nucleic acids into arbitrary geometric shapes with precisely defined properties (FIG. 42). Invented in 2006 by Paul Rothemund (P. W. K. Rothemund, Folding DNA to create nanoscale shapes and patterns, Nature. 440 (2006) 297-302), who constructed two-dimensional (2D) assemblies from bacteriophage-derived genomic DNA, the method has evolved over the past decade and has been applied to form complex 2D and three-dimensional (3D) nanoarchitectures on the 5-450 nanometer-scale with near-quantitative yields and spatial addressability (M. A. Dobrovolskaia, M. Bathe, Opportunities and challenges for the clinical translation of structured DNA assemblies as gene therapeutic delivery and vaccine vectors, WIREs Nanomedicine and Nanobiotechnology. 13 (2021) e1657); (A. A. Arora, C. de Silva, Beyond the smiley face: applications of structural DNA nanotechnology, Nano Rev. & Exp. 9 (2018) 1430976); (K. F. Wagenbauer, C. Sigl, H. Dietz, Gigadalton-scale shape-programmable DNA assemblies, Nature. 552 (2017) 78-83).

In origami nanostructures, a scaffold nucleic acid molecule of up to several thousand bases in length is packed into bundles of double-helical structure. The “glue” that holds these bundles together is Watson-Crick base pairing between complementary sequence segments. Typically, though not necessarily required, hundreds of staple strands are designed to hybridize to two or more segments of the scaffold, thereby creating crosslinks between neighboring helix bundles (FIG. 40).

Staple strands can be manufactured by standard solid-phase oligonucleotide synthesis, which allows on-column incorporation of backbone and nucleotide modifications such as 2′Fluoro (2′F), 2′O-Methyl (2′OMe) and phosphorothioate linkages. Biological methods for staple strand production based on bacteriophages (F. Praetorius, B. Kick, K. L. Behler, M. N. Honemann, D. Weuster-Botz, H. Dietz, Biotechnological mass production of DNA origami, Nature. 552 (2017) 84-87) or rolling circle amplification (C. Ducani, C. Kaul, M. Moche, W. M. Shih, B. Hogberg, Enzymatic production of “monoclonal stoichiometric” single-stranded DNA oligonucleotides, Nat. Methods. 10 (2013) 647-652); (T. L. Schmidt, B. J. Beliveau, Y. O. Uca, M. Theilmann, F. Da Cruz, C.-T. Wu, W. M. Shih, Scalable amplification of strand subsets from chip-synthesized oligonucleotide libraries, Nat. Commun. 6 (2015) 8634) have also been published but are primarily used for the synthesis of unmodified strands.

By introducing modifications in staple strands, the molecular characteristics of origami nanostructures can be tuned. For example, but without limitation, a targeting group can be attached to target specific cells. Similarly, a cell penetrating peptide or lipid can be attached to help overcome certain physical barriers like the endosome. Staple strands modified with 2′F, 2′OMe and/or phosphorothioate linkages may be used to control immunomodulation and serum stability.

Independent of the staple strands, the folding of the nucleic acid into a more compressed structure could make it less immunogenic and less prone to nuclease degradation (in comparison to single stranded mRNA), improving the safety and half-life of the nucleic acid drug. Furthermore, the size and shape of the origami could be designed to direct NA therapeutics to specific organs. For instance, particles with sizes of more than 100 nm are likely to accumulate in the spleen and liver. Hence, the specific folding of the origami gives control over the biodistribution and potential therapeutic targets.

Methods for the Decoration of NA Origamis with Functional Groups

In compositions of the invention, cargo molecules (see for example Table 5) may be attached to nucleic acid origami structures, functional elements, or both via linkers. The attachments may be covalent or non-covalent. The attachments may be reversible. Particularly useful are reversible attachments that bind the cargo molecule to the nanoparticle or functional element while the composition is being transported to a target and then release the cargo molecule from the nanoparticle or functional element when the cargo molecule has been delivered to the target. Examples of reversible linkers that may be used in compositions of the invention include acetals, acid-labile linkages, amino esters, azide-alkyne bonds, biotin-streptavidin linkages, disulfide bonds, dithiopyridyls, enzymatically cleavable linkages, hydrazones, imines, maleic anhydrides, maleimides, nucleotide base pairs, ribozyme linkages, Schiff-base linked imidazoles, thioethers, and triethylene glycol (see for example Table 4).

### Therapeutic Application of Decorated Origamis

Efficient delivery of nucleic acid therapeutics to target cells remains one of the greatest challenges in the field. Naked administration can lead to uptake by macrophages, dendritic cells, and lung epithelial cells (M. Y. T. Chow, Y. Qiu, J. K. W. Lam, Inhaled RNA Therapy: From Promise to Reality. Trends Pharmacol Sci. 41(2020) 715-729). To achieve cellular internalization in other cell types, however, delivery vehicles are required. LNPs are currently the leading choice and the most clinically advanced vehicles. A typical LNP consists of (i) an amino lipid that aids NA encapsulation, cellular uptake, endosomal escape, and improves tolerability, (ii) a phospholipid that stabilizes the bilayer and contributes to endosomal escape, (iii) cholesterol or a sphingolipid for enhanced stability; and (iv) polyethylene glycol (PEG) to reduce nonspecific binding to proteins and increase bioavailability.

### BRIEF DESCRIPTION OF THE DRAWINGS

FIG. 1 is a schematic showing example nucleic acid nanoconstructs (also referred to herein as Mergo(s)). The nucleic acid core nanostructure can be any shape (e.g., square, hexamer), but is presented as a square in this schematic depiction. [A] Modifications to base/sugar/backbone RNA and/or DNA to alter PK/PD properties. [B] RNA and/or DNA (modified and unmodified) oligonucleotide therapeutic or nucleic acids (modified and/or unmodified) that form part of a nucleic acid nanostructure. [C] Linker mechanism to join cargo to RNA and/or DNA. [D] Cargo, e.g., Peptide, oligonucleotide, aptamer and/or small molecule.

FIG. 2 is a schematic showing the location of core modifications. Nucleic acid core nanostructure can be any shape (e.g., square, hexamer), but is presented as a square in this schematic depiction. (i): Modifications to RNA and/or DNA to alter pharmacological properties and/or the position of L-RNA; (ii) RNA and/or DNA (modified and unmodified) oligonucleotide therapeutic or nucleic acids (modified and/or unmodified) that form part of a nucleic acid nanostructure; (iii) Linker mechanism to join cargo to RNA and/or DNA; Cargo molecule, e.g., Peptide, oligonucleotide therapeutic, aptamer and/or small molecule.

FIG. 3 is a schematic showing: Circular combinatorial chains (row A) and linear combinatorial chains (row B) for simplified manufacture of multi payload therapeutics. (i) DNA (e.g., plasmid) or in vitro transcribed RNA; (ii) sense/antisense RNAi strand and/or ASO; (iii) ds RNAi therapeutic; (iv) Cargo molecule, e.g., one or more of siRNA, peptide, small molecule; (v) linker mechanism (e.g., one or more of, click chemistries, complementary binding); (vi) Cleaver sequence.

FIG. 4 is a schematic showing compositions in embodiments of the invention. Row A shows DNA, RNA and/or chimeric DNA/RNA that is both unmodified and modified (to alter stability/pk/pd/ADME, etc.). Row B shows Therapeutic RNA, e.g., ds RNAi, ASO.

FIG. 5 is a schematic showing compositions in embodiments of the invention. Row C shows a linker mechanism. Row D shows a cargo molecule.

FIG. 6 is a schematic depicting nucleic acid variable regions. Therapeutic oligonucleotides can be linked to a nucleic acid of one or more nucleobases that can be shortened in length (bp) or extended to modify the overall size of the entire structure. The variable regions can be therapeutic (or non-therapeutic), contain modified nucleic acids (DNA and/or RNA), both single stranded and double stranded. The variable regions are designed to alter key pharmacological profiles of the construct (PK/PD/ADME/charge/toxicity/hydrophobicity/Protein binding properties, etc.) to enhance the performance of the therapeutic cargo molecule. Additional cargo molecules can be linked to the variable region at the internal and terminal positions. (i): Variable region (ii): Variable region can be extended by adding additional nucleobases (e.g., i vs. ii). (iii): Variable region is made of DNA, RNA and/or chimeric DNA/RNA that is both unmodified and modified (to alter stability/pk/pd/ADME, etc.). (iv): Therapeutic RNA, e.g., ds RNAi, ASO (v): Linker mechanism; and (vi): Cargo molecule.

FIG. 7 is a chart showing the in-silico evaluation of the pharmacological profiles of building blocks to be attached to RNA and/or DNA constructs.

FIG. 8 is a schematic showing triple (v) and two (vi) point branches and linked molecules. (i) NA (RNA and/or DNA), including chemically modified and therapeutic NAs; (ii) NA cargo of different composition to iv; (iii) linker molecule, for example, click chemistry; (iv) NA or non-nucleic acid cargo molecule.

FIG. 9 is a schematic showing triple (vi) and two (vi) point branches and linked molecules linked to another branched unit. (i) NA (RNA and/or DNA), including chemically modified and therapeutic NAs; (ii) NA cargo of different composition to iv; (iii) linker molecule, for example, click chemistry; (iv) NA or non-nucleic acid cargo molecule; (vii) branched units linked together via complementary binding.

FIG. 10 is a schematic showing triple and two-point branches on a nucleic acid nanoparticle. (i) Triple point branching unit; (ii) Two-point branching unit; (iii) non-nucleic acid cargo molecule; (iv) NA (RNA and/or DNA), including chemically modified and therapeutic NAs; (iv) NA cargo of different composition to (iv).

FIG. 11 is a schematic showing the molecular structure of L-RNA as compared to R-RNA.

FIG. 12 is a UV trace at 260 nm of a CuAAC reaction monitorization together with the corresponding trace of the starting material for an oligonucleotide with 12 possible modifications. Each one of the peaks observed in the chromatogram corresponds to the different possible clicked products (confirmed by MS).

FIG. 13 is a UV trace at 260 nm of a click reaction monitorization together with the corresponding trace of the starting material for an oligonucleotide with 19 possible modifications. The full conversion was confirmed with the MS (bottom).

FIG. 14 shows collective MS spectra of selected endosomal escape-mediating peptides.

FIG. 15 shows an anion exchange (IEX) preparative HPLC trace of an RNA-peptide conjugate. The latest eluting fractions were collected as pure material.

FIG. 16 is a schematic depicting small molecule oligophosphate monomers that can be functionalized with PK/PD modulating modifications, or have the modifications integrated directly.

FIG. 17 is a schematic depicting: (A) the PK/PD modulating oligophosphate functionalized with a terminal norbornene. R′ can be any given modification to alter that biological activity of the molecule. (B) is a schematic showing example of an siRNA, or any given therapeutic oligonucleotide, conjugated to a PK/PD modulating oligonucleotide, whereby the 2′ positions of the component nucleotides are modified to change either the charge, Tm, protein binding ability or hydrophobicity. (C) shows an siRNA, or any given therapeutic oligonucleotide, conjugated to a PK/PD modulating oligonucleotide, whereby the 2′ positions of the component nucleotides are modified to change either the charge, Tm, protein binding ability or hydrophobicity. The therapeutic oligonucleotides may be conjugated at either the 5′ or 3′ terminus of the PK/PD modulating strand, or both. The siRNA may be attached to the PK/PD modulating strand via click chemistry of via hybridization.

FIG. 18 depicts some exemplary oligophosphate monomers. These are based around a central amine or serinol core. The R′ group extending from the variable region could be any given click handle or modification to change the biological function of the molecule. A is a norbornene (amine core), B is a norbornene (serinol core), C is a guanine (amine core), D is a histamine-like (amine core).

FIG. 19 is a proposed synthetic route of a norbornene oligophosphate monomer (amine core).

FIG. 20 is a proposed synthetic route of oligophosphate monomers that incorporate PK/PD modulating modifications directly. R could be, but is not limited to, (Z)-N-(N-(6-aminohexyl)-N′-benzoylcarbamimidoyl)benzamide or (4-(aminomethyl)-1H-imidazol-1-yl)methyl pivalate.

FIG. 21 is a proposed synthetic route of oligophosphate monomers with a serinol core. R could be, but is not limited to, (Z)-6-(2,3-dibenzoylguanidino)hexanoic acid, 4-oxo-4-(((1-((pivaloyloxy)methyl)-1H-imidazol-4-yl)methyl)amino)butanoic acid, bicyclo[2.2.1]hept-5-ene-2-carboxylic acid.

FIG. 22 is a schematic showing the use of nucleic acids to modulate PK/PD parameters. Nucleic acid strand can assume any secondary or tertiary structure. Length of each component can vary. Additional cargo molecules can be linked at internal and terminal positions.
(i): RNA and/or DNA (modified and unmodified) (single strand or double or triple helix) of one or more nucleobases;
(ii): RNA and/or DNA (modified and unmodified) of known, unknown or random tertiary structure;
(iii): Therapeutic RNA (or non-therapeutic cargo RNA), e.g., siRNA, ASO, miRNA, saRNA (can be single or double stranded, but is presented as a double line in this schematic depiction).
(iv): Linker mechanism to join cargo to RNA and/or DNA, e.g., IEDDA, SPAAC, CuAAC, hybridisation;
(v): Cargo molecule, e.g., Peptide, aptamer and/or small molecule;
(vi): Modifications to RNA and/or DNA to alter key pharmacological properties (PK/PD/ADME/charge/toxicity/hydrophobicity/Protein binding properties, etc)

FIG. 23 shows atomic force microscopy (AFM) images of nucleic acid nanoparticles unloaded (Mergo X), loaded with x2 siRNA (Mergo Y) and loaded with ×4 siRNA (Mergo Z).

FIG. 24 native PAGE showing the assembly of chemically modified nucleic acid nanoparticles via one-pot thermal anneal protocol in near quantitative yields.

FIG. 25 is a schematic showing size and charge measurements of different nucleic acid nanoparticle constructs and comparison to Lipid Nanoparticles (LNP). Left panel: Surface charge expressed as zeta potential. Right panel: Size (hydrodynamic diameter) plotted as a curve of particle number distribution.

FIG. 26 is a schematic showing the effect of modifications on melting temperature (Tm). The Tm can be calculated by calculating the maximum of the first derivative. The widening of the peak, and the appearance of a second peak in SQ-B indicates destabilization of the structures.

FIG. 27 is a schematic showing the effect of various modifications on protein binding. A) Electrophoretic Mobility Shift Assay (EMSA) example with unmodified Mergo. Increasing % of serum of human serum are added, leading to a shift to lower mobility band (indicating protein binding). EC₅₀ (concentration of serum at which half of Mergo are bound) can be calculated. Human serum concentration is ˜55% in the blood. B) EC₅₀ can be measured for different Mergo in Human serum or Cerebrospinal fluid (CSF). Modifications change the protein binding profile compared to unmodified Mergo or to siRNAs.

FIG. 28 is a schematic showing the effect of various modifications on nuclease susceptibility. Chemical modifications increase half-life of Mergo towards enzymatic degradation compared to unmodified Mergo. (A) Representative graph of degradation of Mergo B (modified) in snake venom phosphodiesterase (SV). Quantification from the gel and fit with one phase decay exponential model. Error bars represent standard deviation (triplicates). (B) Half-life of different Mergo in Snake Venom Phosphodiesterase (SV) (average of triplicates) and RnaseIII (singlicate).

FIG. 29 are graphs showing that covalently linked siRNA cargo molecules retain silencing activity. Data represent mean±SEM of two independent experiments. (A) Comparison of gene silencing activity between free siRNA, siRNA covalently linked to a single RNA strand and siRNA covalently linked to to a Mergo RNA nanoconstruct via IEDDA. Human A549 lung cancer cells were forward transfected with 20 nM of the indicated RNA using lipofectamine 2000, followed by RNA purification and RT-qPCR 48 hours post-transfection. (B) Dose-response analysis of PPIB mRNA levels in A549 cells transfected with M-14 (an RNA nanoconstruct carrying 2 IEDDA-linked PPIB-targeting siRNA cargo molecules). Reverse transfections were performed using lipofectamine RNAiMAX and gene expression was measured 48 hours later using the FastLane Cell SYBR® Green Kit (Qiagen). (C) Effect of various chemical modifications of Mergos on gene silencing activity. A549 cells were reverse transfected with 10 nM of RNA nanoconstructs using lipofectamine RNAiMAX and 48 hours later, cells were lysed and subjected to RT-qPCR with the Luna® Cell Ready One-Step RT-qPCR Kit (New England Biolabs). NTC, non-targeting transfection control (NTC).

FIG. 30 is a graph showing the comparison of gene silencing activity between Mergos loaded with 1× or 2× mono-siRNA, 1× or 2× di-siRNA and unloaded mono- or di-siRNA at equivalent construct concentrations. Data represent mean±SEM of two independent experiments. NTC, non-targeting transfection control (NTC).

FIG. 31 is a graph showing that the effectiveness of endosomal escape peptides depends on the cell uptake pathway. Human MDA-MB-231 breast cancer cells were incubated with 200 nM (free uptake) or transfected with 20 nM RNA constructs loaded with two PPIB-targeting siRNAs and 0, 1 or 2 GFWFG peptides. Gene expression levels were measured 48 hours later by RT-qPCR. Mergos loaded with endosomal escape domain-containing peptides show a trend towards increased gene silencing activity after free uptake (A) but not after lipid-mediated transfection (B). Data represent mean±SD of two independent experiments.

FIG. 32 is a schematic showing uptake and endosomal escape (EE) with Cy3-Gal9 recruitment. (A) Representative images of Hela GFP-GAL9 cells following 24 h incubation with 200 nM Mergo (SQ) or 75 μM chloroquine. Yellow indicates SQ uptake (Cy3-Uptake puncta) and green indicates endosomal escape (GAL9-EE puncta). (B) Sum of Cy3 puncta across a 0-24 h time course dosed with 200 nM SQ. Values were normalized to Cy3 intensity of each SQ and presented as a fold change compared to untreated. (C) Sum of GAL9 puncta across a 0-24 h time course dosed with 200 nM SQ or 75 μM chloroquine. Values were normalized to untreated to obtain fold changes. Data is presented in combined replicates as means±SEM.

FIG. 33 shows biodistribution of Cy3 labeled Mergo in mice on day 7, receiving two injections on day 0 and day 3. Heatmap of the Mean Fluorescent Intensity of different Mergo in liver, kidney, spleen, pancreas, lung, and heart. Values were compared using One-way analysis of variance (ANOVA) with Tukey's multiple comparisons: **P≤0.01, ***P≤0.001, ****P≤0.0001 when compared with the Vehicle control.

FIG. 34 shows biodistribution of Cy3 labelled Mergo in mice on day 7, receiving two injections on day 0 and day 3. Scatter bar graph of the Mean Fluorescent Intensity of different Mergo in lung and heart. Values were compared using One-way analysis of variance (ANOVA) with Tukey's multiple comparisons: **P≤0.01, ***P≤0.001, ****P≤0.0001 when compared with the Vehicle control.

FIG. 35 shows PPIB (Peptidylprolyl Isomerase B) silencing induced by the siRNA delivered by different Mergo in the heart and lung. Values were compared using One-way analysis of variance (ANOVA) with Tukey's multiple comparisons: **P≤0.01, ***P K0.001, ****P≤0.0001 when compared with the Vehicle control.

FIG. 36 shows assessment of toxicity induced in mice. (A) Cytokines were analysed at 2 h post-injection. (B) Biochemical analysis of liver markers. (C) Weight evolution of mice 7 days post-injection.

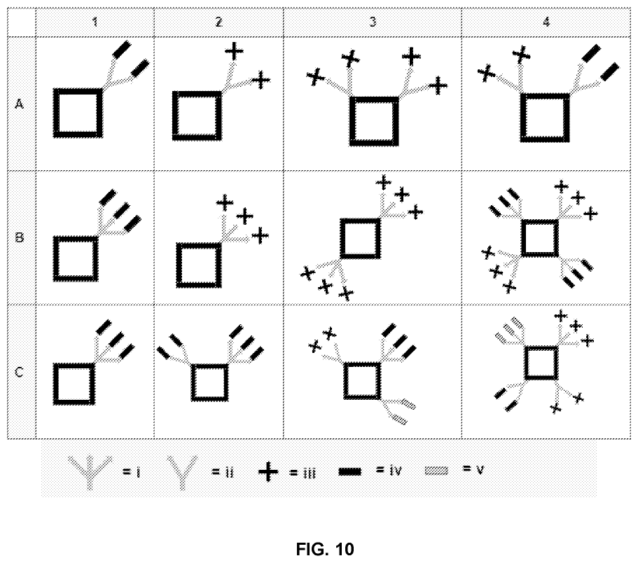

FIG. 37 shows a native analysis showing increased loading Mergo, 1. Mergo without siRNA; 2. Mergo with two single siRNAs; 3. Mergo with one double siRNA; 4. Mergo with two double siRNA; 5. Mergo with four double siRNA. This is a further example of the structures listed in FIG. 10.

FIG. 38 is a proposed synthetic route of a novel serinol-based branching unit for three-way branching FIG. 39 shows an analytical IPLC trace of a dual siRNA linked together by a branching unit. This was purified with a PL-SAX 1000 Å 20 mm prep column using NaClO₄ buffers.

FIG. 40 is a schematic showing an example nucleic acid origami construct composed of an RNA scaffold and unmodified DNA or RNA staple strands.

FIG. 41 is a schematic showing the intracellular dissociation of staple strands from an mRNA origami construct. Upon uptake into a cell (outlined in grey), the staple strands dissociate and release the unpacked, intact mRNA into the cytoplasm. The mRNA is then recognized by ribosomes and translated into the protein.

FIG. 42 is a schematic showing DNA staples binding mRNA in compact configuration through (A) complementary binding.

FIG. 43 is a schematic of an NA origami and sites of cargo attachment, including use of DNA staples for mRNA origami. (A) Staple can be binding (i.e., construct origami) or active (i.e., act as linker to cargo molecule or have function such as IRES or alter charge). (B) Terminal Nucleic Acid to Nucleic Acid bioconjugation (e.g., click). (C) Nucleic Acid linker, e.g., AAAA. Can also act as a cleavage site or IRES position. (D) Bioconjugation, e.g., click chemistry of cargo molecule to nucleic acid on internal backbone, and terminal position. (E) Cargo molecule linked to nucleic acid staple at terminal and/or internal position. (F) Binding Staple

(G) RNA and/or DNA drug, e.g., mRNA. (H) Complementary binding of staple to nucleic acid drug. Modifications to staple free end is designed to alter physiochemical properties. (I) Staple complementary binding from nucleic acid drug to another nucleic acid cargo. (J) Multiple cargoes can be linked onto the origami construct.

FIG. 44 is a schematic showing exemplary arrangements of linking cargo molecules and mRNA origami.

FIG. 45 is a schematic depicting the layout of a 10HB_rectangle mRNA origami.

FIG. 46 is a schematic depicting the layout of a 6HB_tube mRNA origami.

FIG. 47 is a schematic depicting the layout of a 10HB_block mRNA origami.

FIG. 48 is a schematic depicting an oxDNA simulation of the 101113_rectangle mRNA origami.

FIG. 49 is a schematic depicting an oxDNA simulation of the 6HB_tube mRNA origami.

FIG. 50 is a schematic depicting an oxDNA simulation or 10HB_block mRNA origami.

FIG. 51 shows a band-shift assay to confirm assembly of 101113_rectangle, 61113_tube, and 10HB_block on a 2% agarose gel (stained with SYBR Gold).

FIG. 52 shows exemplary AFM images of 10HB_rec. Recorded via an Asylum Research Cypher ES AFM. Found average size: 27.5(±3.3)×23.5(±4.4)×2.0 (10.2) nm. Estimated size: 25.7×23.0×2.3 nm.

FIG. 53 shows four circular mRNA designs. These are either covalently linked at the ends (circular) or are joined via intra-mRNA binding staples (handle).

FIG. 54 is a schematic showing circular combinatorial chains (row A) and linear combinatorial chains (row B) for simplified manufacture of multi payload therapeutics. (i) in vitro transcribed RNA; (ii) single-stranded DNA staples and/or sense/antisense RNAi strand and/or ASO; (iii) chemical modification/conjugate/linker on the staple strand; (iv) Schematic modification on staple strands to improve stability/cell internalization/detachment(v) eukaryotic viral IRES or eukaryotic IRES; (vi) non-coding linker region.

FIG. 55 is a graph showing the results of a fundamental study to determine the chemical nature of the staples that will be used in the origami designs. The antisense oligos were designed to have complementarity to a region in the open reading frame of the Trilink eGFP mRNA. Unmodified DNA antisense oligos were compared to ps-DNA, 2′OMe-DNA and unmodified RNA.

FIG. 56A shows a schematic representation of where the single-stranded staples bind on the scaffold ((i) shows the open reading frames, (ii) shows the 3′UTR); FIG. 56B is a graph which shows the fluorescence, measured 22 h post-transfection, in A549 cells. The data points indicate technical triplicates, the values have been blanked by subtraction of the negative control (only cells) and normalised to the positive control (mRNA, non-hybridised).

FIG. 57 is an RNase H assay with semi-assembled 101113_rectangle variant P with non-modified and 2′OMe-modified staple strands. 2% agarose gel, SYBR Gold stained.

FIG. 58 is a schematic showing the 2′ nucleotide modifications used in this invention.

FIG. 59 is a schematic showing alternative backbone modifications. These include, A) phosphodiester backbone B) phosphorothioate C) alkylated phosphorothioate D) methylphosphonate E) amide F) phosphorodiamidate morpholino oligomers (PMO) G) phosphoramidate H) phosphonoacetate (PACE).

FIG. 60 (left): is an analytical denaturing PAGE gel with batches of C-1.4 (A) and C1.1 (B). (right): Analytical IEX chromatogram of C-1.4 run on a DNAPacP PA200RS with NaCl buffers. Purification of siRNA-modified-RNA conjugates, particularly 2′OMe modified strands, can be non-trivial; A and B tend to co-elute at even very shallow gradients.

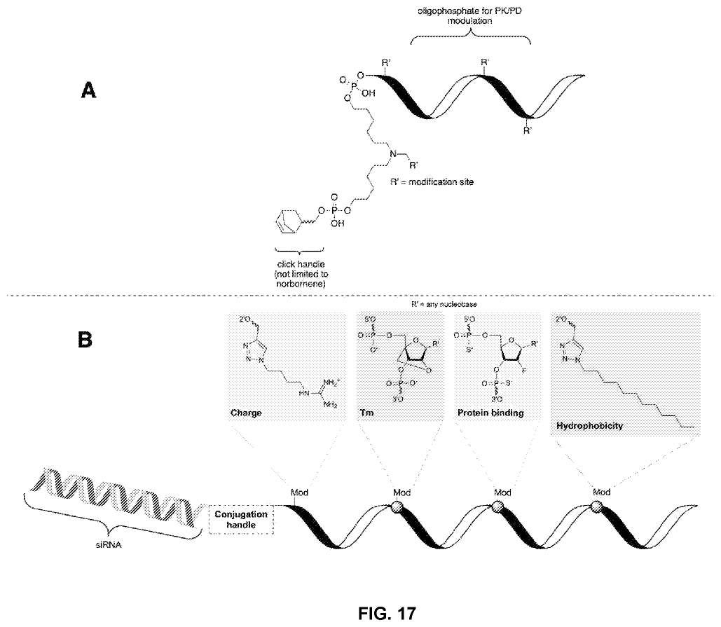

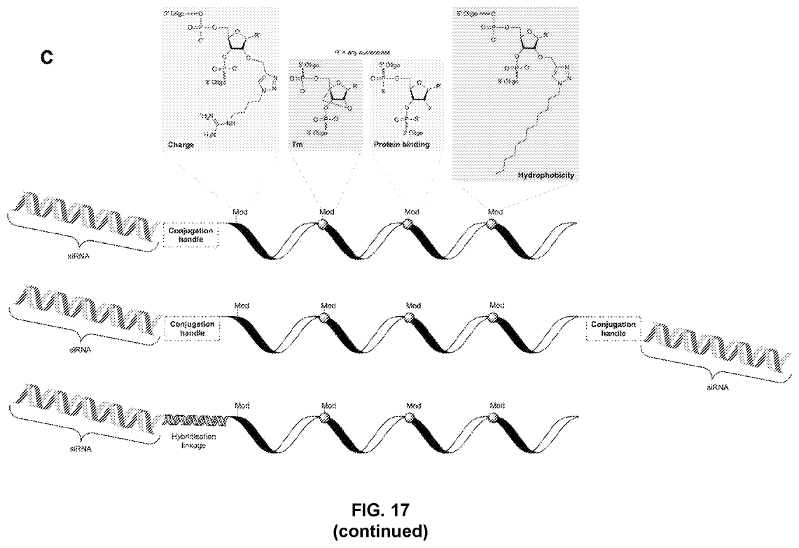

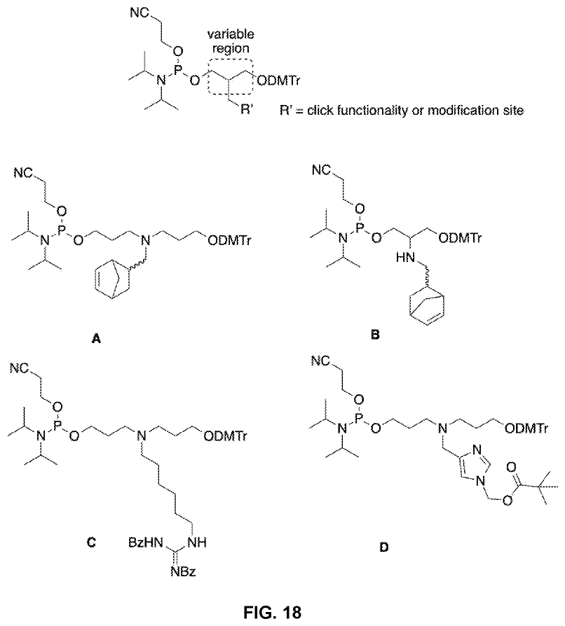

FIG. 61 shows two analytical IEX traces and corresponding denaturing PAGE gels. (A) is a 2′F modified oligonucleotide conjugated to an siRNA via IEDDA. (B) is a 2′OMe modified oligonucleotide conjugated to an siRNA via IEDDA. These molecules can be incorporated into a higher order construct or act as a standalone conjugate, as described in FIG. 17. Chromatographic separation needs to be optimized for each PK/PD modulating tail.

FIG. 62 shows optimization of the assembly of M-1 along with an image of a gel.

FIG. 63 shows quality control of a raw material and an image of a gel.

FIG. 64 shows the effect of various modifications on physicochemical properties.

### DETAILED DESCRIPTION

### Nanoparticles, Including Nucleic Acid Nanoparticles

In certain embodiments, compositions of the invention include nanoparticles. As used herein, “nanoparticle” refers to particles having dimensions that are measured on the nanometer scale. For example, a nanoparticle may have a diameter, length, width, or depth of from 1 to 1000 nm.

RNA nanoparticles are formed from the ordered arrangement of individual RNA molecules having defined secondary structures. RNA molecules form a variety of structural motifs, such as pseudoknots, kissing hairpins, and hairpin loops, that affect both the geometry of the molecule and its ability to form stable interactions with other RNA molecules via base pairing. Typically, individual RNA molecules have double-stranded regions that result from intramolecular base pairing and single-stranded regions that can for base pairs with other RNA molecules or can otherwise bind to other types of molecules.

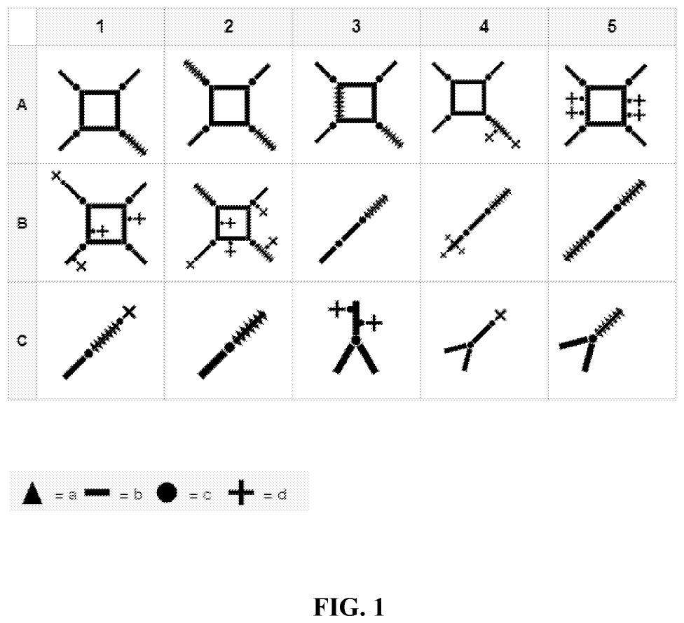

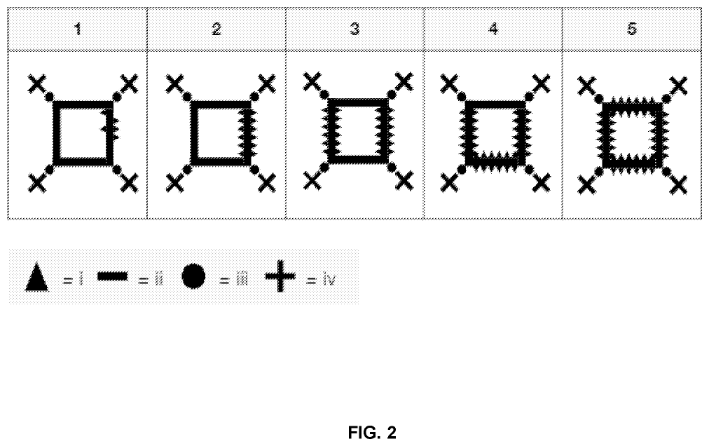

Various RNA nanostructures having ordered two-dimensional or three-dimensional structures are known, including, for example and without limitation, nanoarrays, nanocages, nanocubes, nanoprisms, nanorings, nanoscaffolds, and nanotubes. Nanorings may be symmetrical structures that include 3, 4, 5, 6, 7, 8, or more RNA molecules arrayed around an axis. Thus, nanorings may be trimers, tetramers, pentamers, hexamers, heptamers, oxamers, or higher-numbered polymers. Nanorings may be circular, triangular, square, pentagonal, hexagonal, heptagonal, octagonal, or otherwise polygonal in shape. Other types of RNA nanoparticles, such as sheets, cages, dendrimers and clusters, are also possible and within the scope of the invention. “Nanoscaffold” refers generally to a nanostructure to which other molecules can be attached. RNA nanoparticles of various structural arrangements are described in, for example, WO 2005/003,293; WO 2007/016,507; WO 2008/039,254; WO 2010/148,085; WO 2012/170,372; WO 2015/042,101; WO 2015/196,146; WO 2016/168,784; and WO 2017/197,009, the contents of each of which are incorporated herein by reference. Nucleic acid nanoparticles may contain naturally occurring nucleotides, or they may contain chemically modified nucleotides (for example FIG. 1, FIG. 2). Chemically modified nucleotides are known in the art and described in, for example, WO 2018/118587, the contents of which are incorporated herein by reference. For example, and without limitation, nucleic acid nanoparticles, therapeutics and aptamers may contain one or more of a 2′ fluoro, 2′ O-methyl, 2-thiouridine, 2′-O-methoxyethyl, 2′-amine, 5-methoxyuridine, pseudouridine, 5-methylcytidine, N1-methyl-pseudouridine, locked nucleic acid (LNA), morpholino, and phosphorothioate modification. Other modified nucleotides include 5caC, 5fC, 5hoC, 5hmC, 5meC/5fu, 5meC/5moU, 5meC/thG, 5moC, 5meC/5camU, 5meC, ψ, 5meC/ψ, 5moC/5moU, 5moC/5meU, 5hmC/5meU, me1ψ, 5meC/me1ψ, 5moU, 5camU, m6A, 5hmC/ψ, 5moC/ψ, me6DAP, me4C, 5fu, 5-methoxyuridine, 2-aminoadenine, 2-thiocytosine, 2-thiothymine, 2-thiouracil, 3-methyladenine, 4-thiouracil, 5,6-dehydrouracil, 5-allylcytosine, 5-allyluracil, 5-aminoallylcytosine, 5-aminoallyluracil, 5-bromouracil, 5-ethynylcytosine, 5-ethynyluracil, 5-fluorouracil, 5-hydroxymethylcytosine, 5-hydroxymethyluracil, 5-iodouracil, 5-methylcytosine, 5-methyluracil, 5-propynylcytosine, 5-propynylcytosine, 5-propynyluracil, 5-propynyluracil, 6-O-methylguanine, 6-thioguanine, 7-deaza-8-azaguanine, 7-deazaadenine, 7-deazaguanine, 7-deazaguanine, 8-oxoadenine, 8-oxoguanine, 5-methylcytidine, pseudouridine, inosine, 2′-O-methyladenosine, 2′-O-methylcytidine, 2′-O-methylguanosine, 2′-O-methyluridine, 2′-O-methyl-pseudouridine, 2′-O-methyl 3′-phosphorothioate adenosine, 2′-O-methyl 3′-phosphorothioate cytidine, 2′-O-methyl 3′-phosphorothioate guanosine, 2′-O-methyl 3′-phosphorothioate uridine, a conformationally-restricted nucleotide, and 2′-O-methyl 3′-phosphorothioate pseudouridine. The nucleic acids of the nanoparticles may contain sugar modifications (for example FIG. 1, FIG. 2). For example and without limitation, the nucleic acids of the nanoparticles may contain one or more of 2′MOE, 2′OMe, 2′F, 2-′O-acetalesters, GMEBuOM, AMPrOM, AMEBuOM, PivOM, 2′ amino locked nucleic acids (LNA) modified with amines or peptides mentioned above, 2′-O-[N,N-dimethylamino)ethoxy]ethyl, 2′-N-[N,N-dimethylamino)ethoxy]ethyl, 2′-N-imidazolacetyamide, 2′-O-[3-(guanidinium)propyl], 2′-N-[3-(guanidinium)propyl], 2′-O-[3-(guanidinium)ethyl], 2′-N-[3-(guanidinium)ethyl], 2′-O-(N-(aminoethyl)carbamoyl)methyl, 2′-N-(N-(aminoethyl)carbamoyl)methyl, 2′-O-[N-(2-((2-aminoethyl)amino)ethyl)]acetamide, 2′-N-[N-(2-((2-aminoethyl)amino)ethyl)]acetamide, 2′-N-2,2,3,3,4,4,5,5,6,6,7,7,8,8,8-pentadecafluorooctanamide, 2′-N-imidazolacetamide, 2′-O-imidazole methyl, 2′-N-guanidylbenzylamide, and 4′-C-guanidinincarbohydrazidomethyl, 2′-O-imidazolemethyl, 2′-N-imidazolemethylamine ethyl.

### Use of Phosphoramidite Chemistry to Alter the Physicochemical Characteristics of Nucleic Acid Nanoparticles

In certain embodiments, compositions of the invention include phosphoramidites that provide stimuli-responsive characteristics to the nucleic acid nanoparticle. The first aspect of the invention relates to a compound of formula (I):

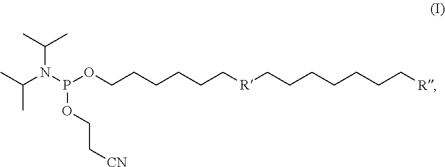

wherein R′ is a functional group that will allow for cleavage of the hydrocarbon chain in response to a range of stimuli. This may include, but is not limited to, a redox-responsive disulfide (J. Winkler, Oligonucleotide conjugates for therapeutic applications, Ther. Deliv. 4 (2013) 791-809), pH responsive hydrazone (N. Ollivier, C. Olivier, C. Gouyette, T. Huynh-Dinh, H. Gras-Masse, O. Melnyk, Synthesis of oligonucleotide-peptide conjugates using hydrazone chemical ligation, Tetrahedron Lett. 43 (2002) 997-999), hydrazine (S. Raddatz, J. Mueller-Ibeler, J. Kluge, L. Wäß, G. Burdinski, J. R. Havens, T. J. Onofrey, D. Wang, M. Schweitzer, Hydrazide oligonucleotides: New chemical modification for chip array attachment and conjugation, Nucleic Acids Res. 30 (2002) 4793-4802), acetal (S. Matysiak, R. Frank, W. Pfleiderer, Acetal oligonucleotide conjugates in antisense strategy, Nucleosides and Nucleotides. 16 (1997) 855-861), or benzoic imine (Y. Wang, Q. Luo, R. Sun, G. Zha, X. Li, Z. Shen, W. Zhu, Acid-triggered drug release from micelles based on amphiphilic oligo(ethylene glycol)-doxorubicin alternative copolymers, J. Mater. Chem. B. 2 (2014) 7612-7619), or ROS-responsive thioketal (Y. Zhang, J. Zhou, S. Ma, Y. He, J. Yang, Z. Gu, Reactive Oxygen Species (ROS)-Degradable Polymeric Nanoplatform for Hypoxia-Targeted Gene Delivery: Unpacking DNA and Reducing Toxicity, Biomacromolecules. 20 (2019) 1899-1913), and wherein R″ is a functional group that will allow for the formation of covalent bonds via reactions selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reactions (spring-loaded reactions), traceless Staudinger ligation (see for example Table 2).

Examples of suitable groups for R′ are provided below:

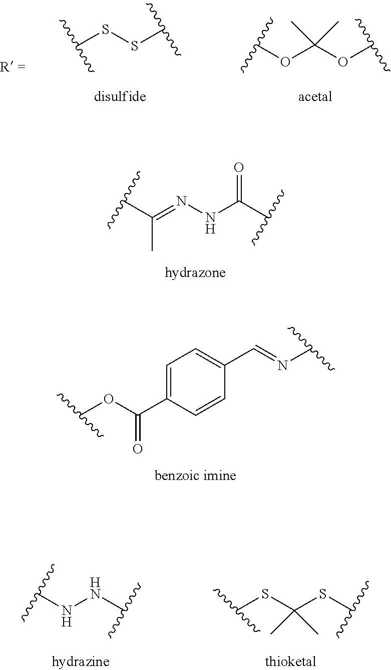

Other suitable R′ groups include variations of the structures illustrated above in which the length of one more aliphatic hydrocarbon chains is altered. For example, an acetal group has the general formula R^{a}₂C(ORb)₂, and both R^{a} and R^{b} may have any length and may be, for example and without limitation, methyl, ethyl, propyl, butyl, pentyl, hexyl, heptyl, octyl, or nonyl groups. Similar substitutions are permitted, where applicable, for any of the other groups illustrated above.

R″ may be synthesized from the group consisting of, but not limited to, ADIBO-PEG4, N-[(1R,8S,9s)-bicyclo[6.1.0]non-4-yn-9-ylmethyloxycarbonyl]-1,8-diamino-3,6-dioxaoctane, (1R,8S,9s)-bicyclo[6.1.0]non-4-yn-9-ylmethanol, bromoacetamido-dPEG®₄-amido-DBCO, bromoacetamido-dPEG®₁₂-amido-DBCO, bromoacetamido-dPEG®₂₄-amido-DBCO, dibenzocyclooctyne-acid, dibenzocyclooctyne-N-hydroxysuccinimidyl ester, dibenzocyclooctyne-PEG4-acid, dibenzocyclooctyne-PEG4-alcohol, dibenzocyclooctyne-PEG4-N-hydroxysuccinimidyl ester, (4-(1,2,4,5-tetrazin-3-yl)phenyl)methanamine hydrochloride, (E)-cyclooct-4-enol, (E)-cyclooct-4-enyl 2,5-dioxo-1-pyrrolidinyl carbonate, 2,5-Dioxo-1-pyrrolidinyl 5-[4-(1,2,4,5-tetrazin-3-yl)benzylamino]-5-oxopentanoate, 5-[4-(1,2,4,5-tetrazin-3-yl)benzylamino]-5-oxopentanoic acid, 5-norbornene-2-acetic acid succinimidyl ester, 5-norbornene-2-endo-acetic acid, methyltetrazine-NHS ester, methyltetrazine-PEG4-NHS ester, TCO PEG4 succinimidyl ester, TCO-amine, tetrazine-PEG5-NHS ester, alkyne-PEG5-acid, (R)-3-amino-5-hexynoic acid hydrochloride, (S)-3-amino-5-hexynoic acid hydrochloride, (S)-3-(boc-amino)-5-hexynoic acid, N-boc-4-pentyne-1-amine, boc-propargyl-Gly-OH, 3-ethynylaniline, 4-ethynylaniline, propargylamine hydrochloride, propargyl chloroformate, propargyl-N-hydroxysuccinimidyl ester, propargyl-PEG2-acid, 3-(4-azidophenyl)propionic acid, 3-azido-1-propanamine, 3-azido-1-propanol, 4-carboxybenzenesulfonazide, O-(2-aminoethyl)-O′-(2-azidoethyl)heptaethylene glycol, O-(2-aminoethyl)-O′-(2-azidoethyl)nonaethylene glycol, O-(2-aminoethyl)-O′-(2-azidoethyl)pentaethylene glycol, azido-dPEG®4(n)acid (where n could be 4, 8, 12, 24), azido-dPEG® (n)-amine (where n could be 7, 11, 23, 35), azido-dPEG®4(n) NHS ester (where n could be 4, 8, 12, 24), azido-dPEG® (n)-TFP ester (where n could be 4, 8, 12, 24, 36), 2-[2-(2-azidoethoxy)ethoxy]ethanol, O-(2-azidoethyl)-O-[2-(diglycolyl-amino)ethyl]heptaethylene glycol, O-(2-azidoethyl)heptaethylene glycol, O-(2-azidoethyl)-O′-methyl-triethylene glycol, O-(2-azidoethyl)-O′-methyl-undecaethylene glycol, 17-azido-3,6,9,12,15-pentaoxaheptadecan-1-amine, 14-azido-3,6,9,12-tetraoxatetradecanoic acid, 11-azido-3,6,9-trioxaundecan-1-amine, bromoacetamido-dPEG® (n)azide (where n could be 3, 11, 23).

Other examples of phosphoramidites included are provided below.

### Formula (II):

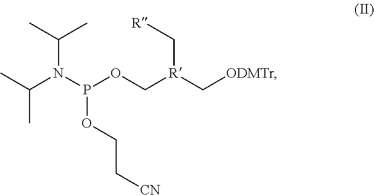 [0108] wherein:
R′ is CH orN; and
R″ is a reactive moiety that permits covalent conjugation.

### Formula (III):

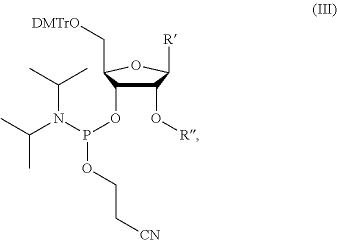 [0112] wherein:
R′ is selected from the group consisting of 2′-deoxyinosine, 2′-deoxynebularine. 3-nitropyrrole 2′-deoxynucleoside, 5′-nitroindole 2′-deoxynucleoside, 6H, 8H-3,4-dihydro-pyrimido[4,5-c][1,2] oxazin-7-one (P), and 2-amino-9-(2-deoxy-β-ribofuranosyl)-6-methoxyaminopurine, adenine, guanine, cytosine, thymine, and uridine; and
R″ is reactive in a reaction a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

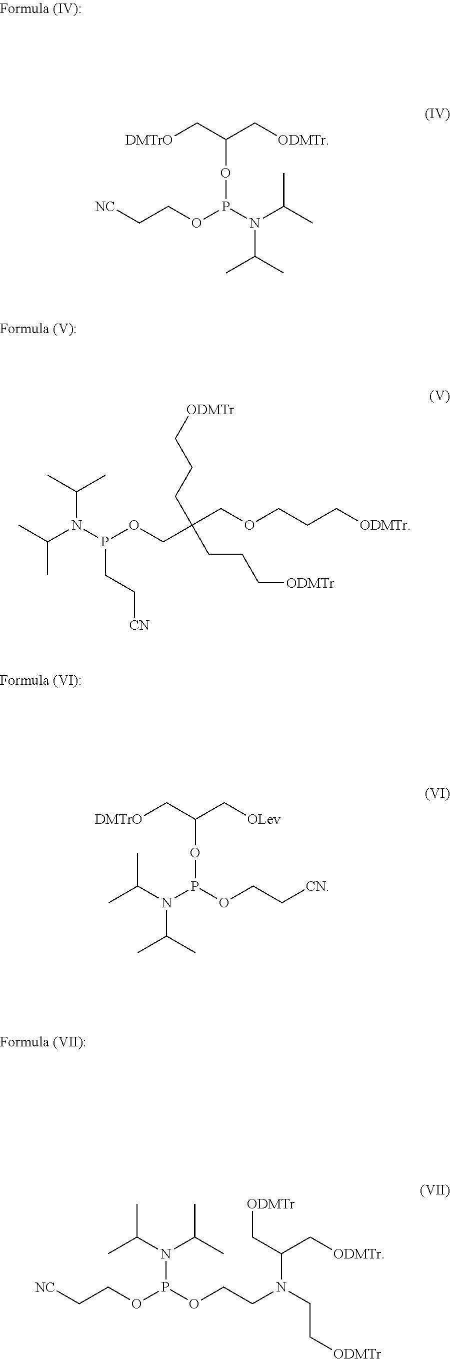

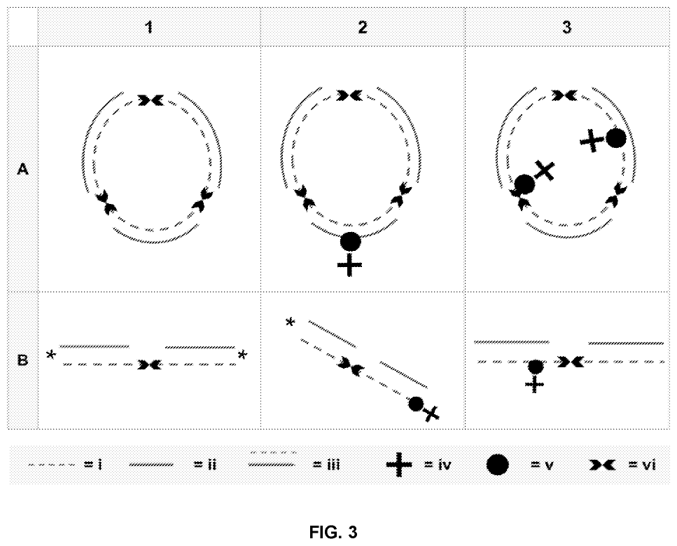

In addition to direct attachment of singular cargo molecules at each attachment point on a nanoparticle, compositions of the present invention may also be used to modify oligonucleotides so that they can be linked to cargo molecules, which are then linked to other cargo molecules (for example FIG. 3).

These linked cargo molecules, also referred to as ‘Combinatorial chains’, could include, but are not limited to, molecules that promote a function and/or biological effect inside or outside a cell (e.g., IRES, ribosomal recruitment, cytokine stimulation), molecules that promote entry into a cell (e.g., peptides, endosomal escape compounds), molecules that bind to target cells (e.g., aptamers, antibodies, ligands), cytotoxic compounds (e.g., cytotoxic nucleosides), molecules that express a gene product inside a cell (e.g., mRNA), chemotherapeutic compounds (e.g., alkylating agents, antimetabolites, topoisomerase inhibitors), molecules that silence or alter a gene inside a cell (e.g., siRNA, miRNA, antisense therapy, lncRNA), CRISPR molecules (e.g., gRNA, Cas9 protein, Cas9 mRNA), small molecule therapies (e.g., protein-tyrosine kinase inhibitors, proteasome inhibitors), proteins, peptides, and diagnostic agents.

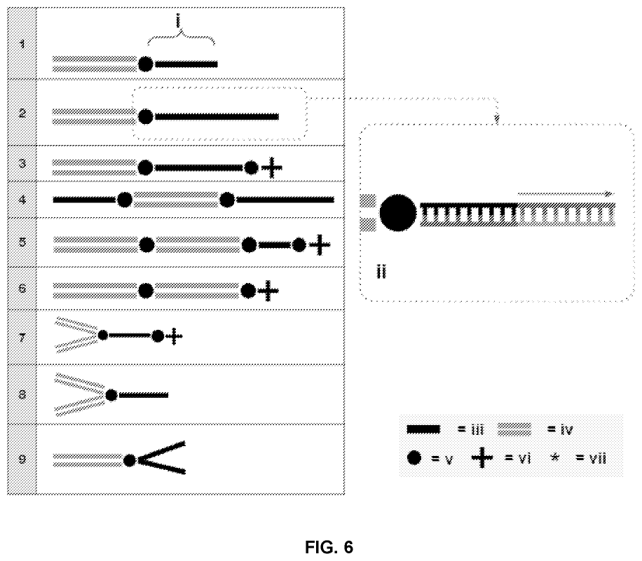

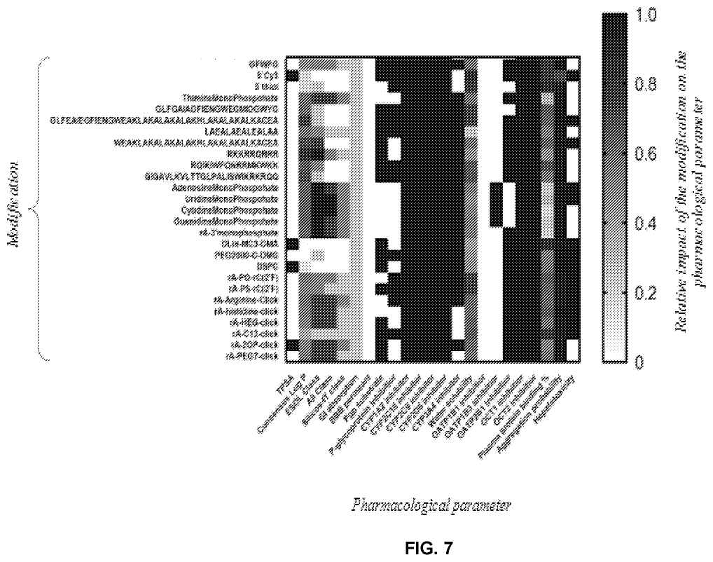

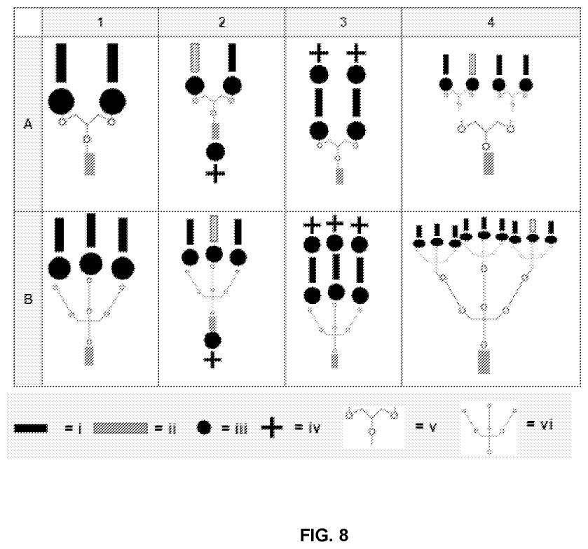

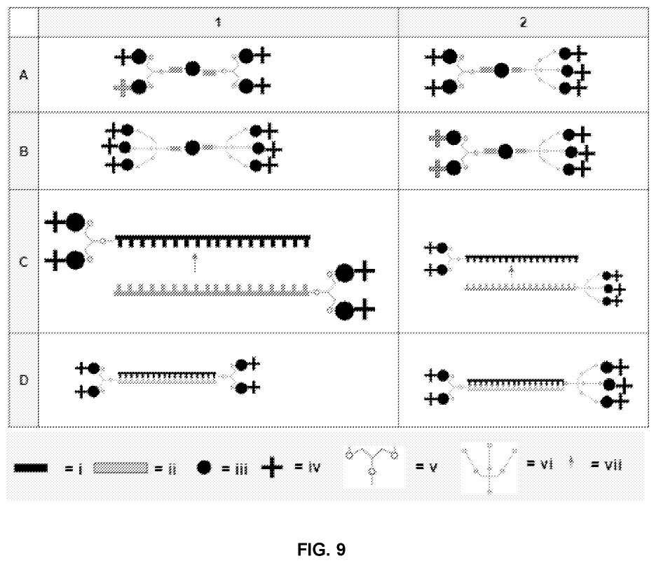

The labile nature of the linkages (see for example Table 4) will allow for these chains to be broken in response to certain stimuli, thereby releasing the payload only when desired. Furthermore, the high reactivity imparted by the reactive handles will allow for comparatively easy assembly of these complex constructs (for example FIG. 6).

In certain embodiments, compositions of the invention include the building blocks that are used to create oligonucleotides and their modifications. DNA and RNA relies on a molecular self-assembly process that is driven by supramolecular interactions between four units that are placed in a defined order. Extending the structural diversity of these recognition units, and even replacing them with alternate functionalities, allows for precise control of the oligo structure at both strand and assembly level (M. Vybornyi, Y. Vyborna, R. Haner, DNA-inspired oligomers: From oligophosphates to functional materials, Chem. Soc. Rev. 48 (2019) 4347-4360); (A. Al Ouahabi, L. Charles, J.-F. Lutz, Synthesis of Non-Natural Sequence-Encoded Polymers Using Phosphoramidite Chemistry, J. Am. Chem. Soc. 137 (2015) 5629-5635). The contents of which are incorporated herein by reference. Chemical alternatives to nucleic acids are indispensable in generating materials that are amenable to a high degree of fine tuning with regards to their physicochemical characteristics.

Nanoparticles may contain any monomeric building blocks that are introduced via phosphoramidite chemistry for direct alteration of its physicochemical properties (for example FIG. 1; FIG. 2). For example, and without limitation, the oligomer unit could contain 2,2-dimethylpropane, propane, tetradecane, N,N-dipropylprop-2-yn-1-amine, methyl dipropylglycyl-L-phenylalaninate, 2-(2-aminopyrimidin-5-yl)-N-((3R,4S)-3-methylheptan-4-yl)acetamide, N-((3R,4S)-3-methylheptan-4-yl)-2-(2,4,6-trioxo-1,3,5-triazinan-1-yl)acetamide, N¹,N⁸-dibutyl-3,5a¹-dihydropyrene-1,8-dicarboxamide, 3,6-di(pent-1-yn-1-yl)-1,5a¹-dihydropyrene, 1,2-dimethoxyethane.

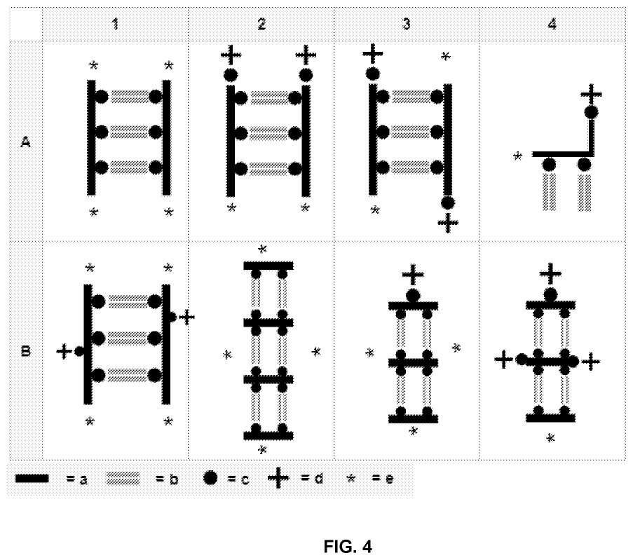

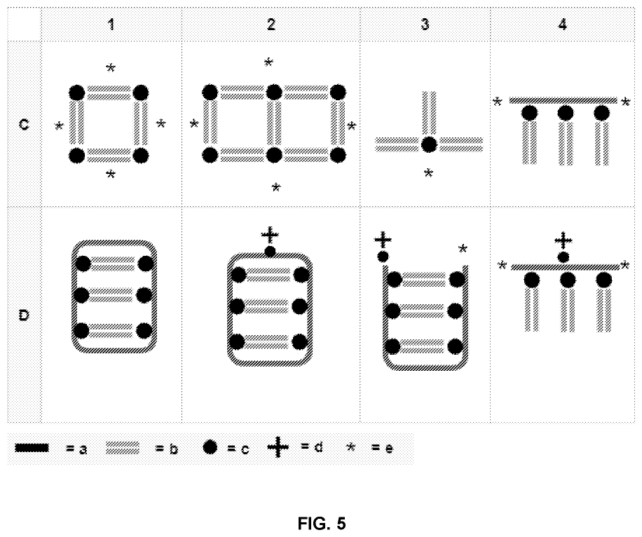

In addition to monomeric building blocks that affect the physical properties of the nanoparticle, the nanoparticle may contain an oligomeric component that has monomeric building blocks functionalized with reactive handles (for example FIG. 4, FIG. 5, FIG. 6). These reactive handles will be compatible with bioorthogonal click chemistries. For example, and without limitation, the oligomer unit could contain any reactive handle protruding from a central amine moiety, which includes, N,N-diethylprop-2-yn-1-amine, N-(bicyclo[2.2.1]hept-5-en-2-ylmethyl)-N-ethylethanamine, 6-(dibenzocyclooctyne)-N,N-diethyl-6-oxohexanamide, (E)-cyclooct-4-en-1-yl (3-ethyl-7,10,13-trioxa-3-azahexadecan-16-yl)carbamate, N,N-diethyl-2-(6-methyl-1,2,4,5-tetrazin-3-yl)ethan-1-amine.

The aforementioned modifications are ideal for alteration of the physicochemical properties of the nucleic acid nanoparticle and would be ideal to add as a tail extending from the central core. In some cases, however, it may be beneficial to incorporate the modifications within the central core itself. These modifications can be incorporated pre- or post-synthetically and would usually require a functionalized nucleotide so that the appropriate conjugation chemistries can occur. In our prior invention (SIX-003/00US 34514/9), we outlined the reactive handles needed for several click chemistry strategies at both strand and nucleotide level. These modifications were on the canonical bases adenine (A), cytosine (C), guanine (G), and thymine (T)/uridine (U). From a manufacturing perspective, the synthesis of multiple modifications on multiple bases would be costly and time consuming. Therefore, it would be beneficial to choose a “universal” base onto which these modifications are attached. Universal bases are used to reduce the degeneracy of the four canonical bases. The most widely used are 2′-deoxyinosine and 2′-deoxynebularine. 3-Nitropyrrole 2′-deoxynucleoside and 5′-nitroindole 2′-deoxynucleoside are also used to some extent. Unfortunately, all these bases have a destabilizing effect on DNA/RNA duplexes, so the search for a true universal base is still widely underway. 6H, 8H-3,4-dihydro-pyrimido[4,5-c][1,2] oxazin-7-one (P) and 2-amino-9-(2-deoxy-β-ribofuranosyl)-6-methoxyaminopurine (dK) show considerable promise as degenerate bases and can effectively H-bond to A or G and C or T, respectively ((P. K. T. Lin, D. M. Brown, Synthesis and duplex stability of oligonucleotides containing cytosine-thymine analogues, Nucleic Acids Res. 17 (1989) 10373-10383); (D. M. Brown, P. K. Thoo Lin, Synthesis and duplex stability of oligonucleotides containing adenine-guanine analogues, Carbohydr. Res. 216 (1992) 129-139)).

The composition may include variants of any given universal base. The base will be attached to a chemically or enzymatically modified nucleotide. Universal nucleotides may be modified on the sugar, at the 2′ position, on the phosphate, or on the universal base (for example FIG. 1, FIG. 2). For example, and without limitation, nucleic acid nanoparticles may be synthesized with any of the following phosphoramidites (with any given variation in the linker length at the 2′ position): (2R,3R,4R,5R)-2-((bis(4-methoxyphenyl)(phenyl)methoxy)methyl)-5-(7-oxo-3,4,7,8-tetrahydro-6H-pyrimido[4,5-c][1,2]oxazin-6-yl)-4-(prop-2-yn-1-yloxy)tetrahydrofuran-3-yl (2-cyanoethyl) diisopropylphosphoramidite, (2R,3R,4R,5R)-4-(bicyclo[2.2.1]hept-5-en-2-ylmethoxy)-2-((bis(4-methoxyphenyl)(phenyl)methoxy)methyl)-5-(7-oxo-3,4,7,8-tetrahydro-6H-pyrimido[4,5-c][1,2]oxazin-6-yl)tetrahydrofuran-3-yl (2-cyanoethyl) diisopropylphosphoramidite, (2R,3R,4R,5R)-2-((bis(4-methoxyphenyl)(phenyl)methoxy)methyl)-4-(2-(((E)-cyclooct-4-en-1-yl)oxy)ethoxy)-5-(7-oxo-3,4,7,8-tetrahydro-6H-pyrimido[4,5-c][1,2]oxazin-6-yl)tetrahydrofuran-3-yl (2-cyanoethyl) diisopropylphosphoramidite; (2R,3R,4R,5R)-4-(2-aminoethoxy)-2-((bis(4-methoxyphenyl)(phenyl)methoxy)methyl)-5-(7-oxo-3,4,7,8-tetrahydro-6H-pyrimido[4,5-c][1,2]oxazin-6-yl)tetrahydrofuran-3-yl (2-cyanoethyl) diisopropylphosphoramidite; (2R,3R,4R,5R)-2-((bis(4-methoxyphenyl)(phenyl)methoxy)methyl)-4-(2-mercaptoethoxy)-5-(7-oxo-3,4,7,8-tetrahydro-6H-pyrimido[4,5-c][1,2]oxazin-6-yl)tetrahydrofuran-3-yl (2-cyanoethyl) diisopropylphosphoramidite.

### Cargo

TABLE 1
List of peptides to be incorporated into compositions of the invention.

| No. | Peptide | Length | Application |
|---|---|---|---|
| 1 | GFWFG | 5 | Endosomal escape |
| 2 | GLFGAIAGFIENGWEGMIDGWYG | 23 | Endosomal escape |
| 3 | GLFEAIEGFIENGWEGMIDGWYG | 23 | Endosomal escape |
| 4 | LAEALAEALEALAA | 14 | Endosomal escape |
| 5 | WEAKLAKALAKALAKHLAKALAKA | 30 | Endosomal escape |
| | LKACEA | | |
| 6 | Poly (Arg) | 2-20 | Change in charge and |
| | | | pKa-PK and PD |
| | | | properties |
| 7 | Poly (Glu) | 2-20 | Change in charge and |
| | | | pKa-PK and PD |
| | | | properties |
| 8 | Poly (His) | 2-20 | Change in charge and |
| | | | pKa-PK and PD |
| | | | properties |
| 9 | Poly (Leu) | 2-20 | Hydrophobic- |
| | | | Change in PK/PD |
| | | | properties |

mRNA Origami

Nucleic acid (NA) therapies aim to cure genetic or acquired diseases caused by aberrant gene expression. Broadly, the therapeutic approaches developed to date can be classified into three main categories. The first category, gene therapy, involves the introduction of corrective genetic material to restore the expression of a missing or defective gene. The second strategy, RNA-based therapy, involves delivery of nucleic acids that reduce the expression levels of defective or overexpressed messenger RNAs (mRNAs), or alternatively provide functional mRNA molecules to increase the expression levels of underexpressed or missing proteins. The third approach, gene editing, allows scientists to correct mutations in endogenous DNA or mRNA sequences.

Compared with DNA gene therapies, the use of RNA therapeutics is considered markedly safer. Not only is there no risk of stable genome integration, but RNA also possesses a short half-life in vivo and is readily degraded by ribonucleases (RNases), ensuring its activity is non-permanent. Moreover, due to the predominantly cytoplasmic localization of RNA, transport across the nuclear membrane is not required, which facilitates delivery. The FDA approval of six antisense oligonucleotides (ASOs), four small interfering RNA (siRNA) therapeutics and the recent success of two mRNA vaccines against COVID-19 demonstrates the therapeutic potential of RNA drugs and, in particular, has put mRNA drugs into spotlight.

Messenger RNA is a temporary copy of genetic information that is copied from DNA and translated into a protein. Mature mRNA is a single-stranded polynucleotide with an average length of 2,000-2,500 bases (T. Ota, et al., Complete sequencing and characterization of 21,243 full-length human cDNAs, Nat Genet. 36 (2004), 40-45). It is characterized by a 5′ 7-methylguanosine cap (m7G), which protects the mRNA from degradation and promotes translation initiation, 5′ and 3′ untranslated regions (UTRs) that flank the protein-coding open-reading frame (ORF), and a 3′ polyA-tail that regulates mRNA stability. Synthetic mRNA can be produced by in vitro transcription (IVT). To prevent immunostimulation, modified nucleobases are introduced during IVT.

Despite the advantages of nucleic acid drugs and continuous progress in the field, mRNA delivery in vivo remains a major challenge. The intrinsically negative charge of mRNA prevents its translocation across negatively charged cell membranes. Moreover, mRNA needs to be protected from enzymatic degradation by ubiquitously expressed RNases. Thirdly, due to mRNA's large size of approximately 105-106 Da, encapsulation in delivery vehicles is more difficult to achieve than for smaller payloads (K. A. Hajj, K. A. Whitehead, Tools for translation: non-viral materials for therapeutic mRNA delivery. Nat. Rev. Mater. 2 (2017) 17056) (C. Zeng, C. Zhang, P. G. Walker, Y. Dong, Formulation and Delivery Technologies for mRNA Vaccines, in: Current Topics in Microbiology and Immunology, Springer, Berlin, Heidelberg, 2020).

Various delivery strategies have been developed to overcome these bottlenecks. (1) The most clinically advanced systems are lipid nanoformulations such as liposomes and lipid nanoparticles that encapsulate the mRNA in a hydrophilic interior surrounded by a protective outer layer of lipids. Although efficacious and successful in the clinics, these delivery vehicles are often associated with toxicity and immunogenicity in vivo, which can be mitigated by using ionizable over cationic lipids. Furthermore, they often provide only limited control over particle size, may suffer from high batch-to-batch variability, and display low encapsulation efficiencies. (2) Viral lentiviruses, adeno-associated viruses and virus-like replicon particles have also been employed as nucleic acid carriers. Whilst allowing efficient cytoplasmic delivery of mRNAs, their application is limited by unwanted immune responses and issues with large-scale production. (3) Cationic polymer shuttles deliver nucleic acids into the cytosol via electrostatic interactions. However, their use is associated with toxicities related to high molecular weight, highly branched formulations, and aggregation. (4) Other delivery strategies based on transcript-activated matrices, exosomes, peptides and nanoemulsions have been reviewed (K. A. Hajj, K. A. Whitehead, Tools for translation: non-viral materials for therapeutic mRNA delivery, Nat. Rev. Mater. 2 (2017) 17056); (C. Zeng, C. Zhang, P. G. Walker, Y. Dong, Formulation and Delivery Technologies for mRNA Vaccines, in: Current Topics in Microbiology and Immunology, Springer, Berlin, Heidelberg, 2020); (T. C. Roberts, R. Langer, M. J. A. Wood, Advances in oligonucleotide drug delivery, Nat Rev Drug Discov 19 (2020), 673-694); (S. Uchida, F. Perche, C. Pichon, and H. Cabral, Nanomedicine-Based Approaches for mRNA Delivery, Molecular Pharmaceutics 17 (2020), 3654-3684).

Overall, there remains an unmet need in the art for improved nucleic acid compositions suitable for general clinical use. Current research is largely devoted to fine tuning the composition of delivery vehicles and enhancing the tolerability thereof. The present invention, in contrast, describes the use of NA nanotechnology to precisely tweak the characteristics of the NA drug molecule itself. By exploiting the programmability of NA base-pairing, compact structures of controlled shape, size and complexity can be formed. Compaction occurs through a process of molecular self-folding termed origami, in which a single-stranded DNA or RNA molecule (scaffold, e.g., an mRNA) hybridizes to one or more DNA or RNA molecules, for example hundreds of short complementary 20-60-mer staple strands. (P. Rothemund, Folding DNA to create nano-scale shapes and patterns, Nature. 440 (2006) 297-302); (S. Douglas, H. Dietz, T. Liedl, et al. Self-assembly of DNA into nanoscale three-dimensional shapes. Nature 459 (2009) 414-418); (N. Seeman, “Nanomaterials based on NA”, An. Ref Biochem. 79 (2010) 65-87); (X. Qi, X. Liu, L. Matiski, R. R. Del Villar, T. Yip, F. Zhang, S. Sokalingam, S. Jiang, L. Liu, H. Yan, and Y. Chang, ACS Nano 14 (2020), 4727-4740).

The present invention provides compositions and methods that can be used to reversibly compact nucleic acids into defined origami shapes and sizes with tunable pharmacokinetic and pharmacodynamic (PK/PD) properties. The compactness of the origami structure may protect the therapeutic moiety against chemical, biochemical or mechanical stresses and increase its resistance against nucleases. Size, rigidity, and shape of the construct can be varied to modulate packaging and achieve a favorable PK/PD profile. In addition, each of the origami building blocks can be selectively modified to tune the PK/PD properties of the origami structure, such as serum stability, biodistribution and cellular uptake. Upon internalization by living cells, the construct may dissociate and release the decompacted therapeutic molecule(s) (FIG. 41). The compositions and methods of the invention are particularly useful for, but not limited to, the delivery of therapeutic mRNA molecules into live cells for treating diseases caused by the deficiency or insufficient levels of a functional protein. The compositions and methods of the invention are also useful for the delivery of mRNA vaccines into live cells to induce expression and presentation of antigens for immune stimulation. The principles outlined in this invention can be extended to multiple mRNAs to create synergistic effects. This invention can be applied to other types of therapeutics, as the composition can serve as a platform for the conjugation with small molecules, peptides, antibodies and any other therapeutic.

Other objects, features, and advantages of the present invention will be apparent to one of skill in the art from the following detailed description and figures.

TABLE 2
Overview over chemical conjugation reactions between reactive
groups X and Y.

| Row | | | | |
|---|---|---|---|---|
| id | Chemistry | X | Y | Linkage |
| 1 | CuAAC | alkyne | azide | triazole |
| 2 | SPAAC | cycloalkyne | azide | triazole |
| 3 | RuAAC | alkyne | azide | triazole |
| 4 | IEDDA | tetrazine | dienophile/ | pyridazine |
| | | | olefin | |
| 5 | NHS/amine | NHS ester | amine | amide |
| 6 | thiol/ | maleimide | thiol | thioether |
| 7 | maleimide | alkoxyamine/ | aldehyde or | oxime/ |
| | oxime/ | hydrazine | ketone | hydrazone |
| | hydrazone | | | |
| 8 | SuFEx | sulfonyl | amine, | sulfonamide, |
| | | fluoride, | aryl silyl | sulfonate or |
| | | fluorosulfate | ether | sulfate |
| 9 | SPANC | strained alkyne | nitrone | isoxazoline |
| 10 | thiol-ene | thiol | alkene, | thioether |
| | radical addition | | acrydite | |
| 11 | thiol-yne | thiol | alkyne | alkenyl sulfide |
| | addition | | | |
| 12 | thiol-isocyanate | thiol | isocyanate | thiourethane |
| 13 | thiol-epoxy | thiol | epoxide | thioether |
| 14 | amine-epoxy | amine | epoxide | hydroxyl amine |
| 15 | Staudinger | phosphine, | azide | imino- |
| | ligation | phosphite | | phosphorane |
| 16 | Disulphide | thiol | thiol | disulphide |
| 17 | Carbodiimide | carboxylate or | amine | amide or |
| | activation | phosphate | | phosphoramidate |

X = CuAAC = copper(I)-catalyzed alkyne-azide cycloaddition,
SPAAC = strain-promoted azide-alkyne cycloaddition,
RuAAC = ruthenium-catalyzed azide-alkyne cycloaddition,
IEDDA = inverse electron demand Diels-Alder reaction,
NHS = N-hydroxysuccinimide,
SuFEx = sulfur(VI) fluoride Exchange,
SPANC = strain-promoted alkyne-nitrone cycloaddition

TABLE 3
Chemoenzymatic conjugation between functional groups X and Y.

| Row id | Position | Enzyme | X | Y | Linkage |
|---|---|---|---|---|---|
| 1 | 5′ | T4 poly- | phosphate or | DNA 5′OH | phospho- |
| | | nucleotide kinase | phosphate | | monoester |
| | | | isotopes | | |
| 2 | internal/ | DNA or RNA | modified | DNA 3′OH | phosphodiester |
| | 5′/3′ | polymerase, | nucleotides | RNA 3′OH | |
| 3 | 3′ | Terminal | modified | DNA 3′OH | phosphodiester |
| | | transferase | nucleotides | | |
| 4 | 3′ | Ligase | DNA or RNA | DNA or | phosphodiester |
| | | | 3′OH | RNA | |
| | | | | 5′phosphate | |
| 5 | internal/ | Methyltransferase | aziridine or N- | nucleotide | alkyl |
| | 5′/3′ | (e.g., SMILing) | mustard | (e.g., amino | |
| | | | mimics of | group of | |
| | | | cofactor | adenosine) | |
| 6 | internal/ | Methyltransferase | double | nucleotide | alkyl |
| | 5′/3′ | (e.g., for mTAG) | activated | (e.g., amino | |
| | | | AdoMet | group of | |
| | | | analogues | adenosine) | |
| 7 | internal | β-glucosyl- | UDP glucose | 5-hydroxy- | glucosyl |
| | | transferase | functionalized | methyl- | |
| | | | with amine, | cytosine | |
| | | | ketone or | | |
| | | | azide group | | |
| 8 | internal | tRNA^{Ile2}_ | agmatine | tRNA^{Ile2} tag | agmatidine |
| | | agmatidine | analog | | |
| | | synthetase (Tias) | | | |
| 9 | internal | tRNA guanine | preQ1 | tRNA^{Tyr} tag | glycosyl |
| | | transglycosylase | analogue | | |
| 10 | internal/ | Ribozyme | electrophile | nucleophile | N/A |
| | 5′/3′ | | | | |
| 11 | internal | DNAzyme | functionalized | RNA 2′OH | 2′,5′-linked |
| | | | NTP | | phosphodiester |
| 12 | N- or C-term | Modified O⁶- | O⁶- | SNAP-tag | thioether |
| | | alkylguanine-DNA- | benzylguanine | labelled | |
| | | alkyltransferase | (BG) | protein | |
| | | (AGT) | derivative | | |
| 13 | N- or C-term | Modified O⁶- | O²- | CLIP-tag | thioether |
| | | alkylguanine-DNA- | benzylguanine | labelled | |
| | | alkyltransferase | (BG) | protein | |
| | | (AGT) | derivative | | |
| 14 | N- or C-term | modified | Halo Tag | Halo-tag | ester |
| | | haloalkane | ligand | labelled | |
| | | dehalogenase | (chloroalkane | protein | |
| | | | linker) | | |
| 15 | C-term | protein | FPP | Cys near C- | thioether |
| | | farnesyltransferase | analogues | terminus | |
| | | (PFTase) | | | |
| 16 | internal/ | transglutaminase | acyl moiety of | primary | isopeptide |
| | N- or C-term | | glutamine | amine | |
| | | | residue | | |
| 17 | C-term | sortase A (SrtA) | aminoglycine | signal | amide |
| | | | or amine | peptide | |
| | | | | LPXTG | |
| 18 | internal | formylglycine- | aminooxy- or | CXPXR | oxime |
| | | generating enzyme | hydrazide- | pentapeptide | |
| | | (FGE) | bearing | tag | |
| 19 | 5′, | HUH-endonuclease | oligo bearing | HUH tagged | phospho- |
| | N- or C-term | domain | target | protein | tyrosine |
| | | | sequence | | |

SMILing = sequence-specific methyltransferase-induced labeling,
NTP = nucleoside triphosphate,
mTAG = methyltransferase- directed transfer of activated groups,
AdoMet = S-Adenosyl-1-methionine,
N- or C-term = N- or C-terminal

TABLE 4
Linker methodologies

| | | Functional | Functional | | |
|---|---|---|---|---|---|
| | | group of | group of | | |
| Row id | Linker | reactant A | reactant B | Cleavability | Charge |
| 1 | Nucleic acid | 3′ hydroxyl | 5′ phosphate | nuclease sensitive | negative |
| 2 | SPP | amine | thiol | redox sensitive | neutral |
| 3 | SPDB/ | amine | thiol | redox sensitive | neutral/ |
| | sulfo-SPDB | | | | negative |
| 4 | SPDP | amine | thiol | redox sensitive | neutral |
| 5 | AcBut | amine | hydrazine | acid sensitive | neutral |
| 6 | Hydrazine | carboxyl | aldehyde | acid sensitive | neutral |
| 7 | Hydrazide | variable | aldehyde | acid sensitive | neutral |
| | | (e.g., alkyne) | | | |
| 8 | Hydrazone or | variable | variable | acid sensitive | neutral |
| | N-acyl hydrazone | (e.g., alkyne) | (e.g., thiol) | | |
| 9 | Cis-aconityl | amine | hydroxyl | acid sensitive | neutral |
| | | (EDC-NHS) | (carbodiimide) | | |
| | | | or amine | | |
| 10 | Acetal | variable | variable | acid sensitive | neutral |
| | | (e.g., | (e.g., | | |
| | | hydroxyl) | hydroxyl) | | |
| 11 | Imine | variable | variable | acid sensitive | neutral |
| | | (e.g., | (e.g., amine) | | |
| | | aldehyde) | | | |
| 12 | Orthoester | variable | variable | acid sensitive | neutral |
| | | (e.g., | (e.g., thiol) | | |
| | | hydroxyl) | | | |
| 13 | Trans-cyclooctene | variable | variable | tetrazine sensitive | neutral |
| | | (e.g., azide) | (e.g., amine) | (via IEDDA) | |
| 14 | Peptide | variable | variable | protease sensitive | variable |
| | e.g., Phe-Lys, Val- | (e.g., amine) | (e.g., imide) | | |
| | Ala, Val-Ci, Glu-Val- | | | | |
| | Cit | | | | |
| 15 | Peptide-PABC | amine | amine | protease sensitive | variable |
| | ( Phe-Lys-PABC, Val- | | | | |
| | Ci-PABC, cBu-Cit- | | | | |
| | PABC) | | | | |
| 16 | β-glucuronide | thiol | variable | β-glucuronidase | neutral, |
| | | | (e.g., amine, | sensitive | hydrophilic |
| | | | phenol) | | |
| 17 | β-galactoside | thiol | variable | β-galactosidase | neutral, |
| | | | (e.g., carboxy) | sensitive | hydrophilic |
| 18 | pyrophosphate | variable | variable | pyrophosphatase/ | negative |
| | | (e.g., azide) | (e.g., | acid phosphatase | |
| | | | hydroxyl) | sensitive | |
| 19 | SMCC/ | amine | thiol | non-cleavable | neutral/ |
| | sulfo-SMCC | | | | negative |
| 20 | mc | amine | thiol | non-cleavable | neutral |
| 21 | PEG4Mal | variable | thiol | non-cleavable | neutral, |
| | | (e.g., thiol, | | | hydrophobic |
| | | alkene) | | | |
| 22 | Iodoacetyl | thiol | | non-cleavable | positive |
| | polyhistidine | | | | |
| 23 | HyNic-4FB | amine | amine | non-cleavable | neutral |
| 24 | SMPB | amine | thiol | non-cleavable | neutral |
| 25 | SIAB/ | amine | thiol | non-cleavable | neutral/ |
| | sulfo-SIAB | | | | negative |
| 26 | EDC | carboxyl | amine | non-cleavable | positive, |
| | | | | | zero-length |

AcBut = 4-(4-acetylphenoxy)butanoic acid
EDC = 1-ethyl-3-(3-dimethylaminopropl)-carbodiimide hydrochloride
EPP = farnesyl diphosphate, formylbenzoate,
PABC = para-aminobenzyl carbamate,
HyNic & 4FB = succinimidyl 6-hydrazinonicotinate acetone hydrazone & succinimidyl 4-formylbenzoate
mc = maleimidocaproyl linker,
mcc = maleimidomethyl cyclohexane-1-carboxylate,
SMPB = (succinimidyl 4-(p-maleimidophenyl)butyrate),
SIAB = (succinimidyl (4-iodoacetyl)aminobenzoate),
SPP = N-succinimidyl-4-(2-pyridyldithio)pentanoate,
SPDB = N-succinimidyl-4-(2-pyridyldithio)butyrate,
SPDP = N-Succinimidyl 3-[2-pyridyldithio]-propionate
SMCC = N-succinimidyl-4-(N-maleimidomethyl)cyclohexane-1-carboxylate,
EDC = 1-ethyl-3-(3-dimethylaminopropl)-carbodiimide hydrochloride
EPP = farnesyl diphosphate, formylbenzoate,
PABC = para-aminobenzyl carbamate,
HyNic & 4FB = succinimidyl 6-hydrazinonicotinate acetone hydrazone & succinimidyl 4-formylbenzoate
mc = maleimidocaproyl linker,
mcc = maleimidomethyl cyclohexane-1-carboxylate,
SMPB = (succinimidyl 4-(p-maleimidophenyl)butyrate),
SIAB = (succinimidyl (4-iodoacetyl)aminobenzoate),
SPP = N-succinimidy1-4-(2-pyridyldithio)pentanoate,
SPDB = N-succinimidyl-4-(2-pyridyldithio)butyrate,
SPDP = N-Succinimidyl 3-[2-pyridyldithio]-propionate
SMCC = N-succinimidy1-4-(N-maleimidomethyl)cyclohexane-1-carboxylate

TABLE 5
Cargo types

| Row id | Cargo type | Function | Example |
|---|---|---|---|
| 1 | Nucleic acid | therapeutic | mRNA, microRNA, siRNA, shRNA, saRNA, |
| | | | antimir, ASO, gapmer, splice-switching |
| | | | oligomer, aptamer, gRNA (CRIPSR, ADAR), |
| | | | plasmid, ribozyme, BERA, |
| | | targeting | aptamer, spiegelmer |
| | | recruiting | poly A motif, AREs, CREs, IREs, restriction |
| | | | site, nuclear localization signal, G quadruplex, |
| | | | pseudoknot |
| | | reporting | barcode sequence, hybridization probe, G |
| | | | quadruplex, ribozyme |
| | | packaging/ | hybridization arm, self-dimerization domain, |
| | | carrier | toehold, RNA nanoparticle, DNA/RNA origami |
| | | | construct |
| 2 | Peptide | therapeutic | insulin, adrenocorticotropic hormone (ACTH), |
| | | | calcitonin, oxytocin, vasopressin, octreotide, |
| | | | exenatide, carfilzomib, voclosporin, glatiramer, |
| | | | dulaglutide, anti-microbial peptides, leuprolide, |
| | | | goserelin, and octreotide |
| | | targeting | receptor binding (e.g., iRGD, Cilengitide, |
| | | | SFITGv6), ECM targeting (e.g., CNGRC, |
| | | | RGDechi, DAG, ZD2, CSG), tumor associated |
| | | | macrophage targeting (e.g., RP-182, M2pep, |
| | | | mUNO), bicyclic peptides |
| | | cell penetrating/ | Penetratin, R8, TAT, Transportan, Xentry, |
| | | endosomal escape | MAP, IVV-14 |
| | | Packaging/ | cationic peptides (NA binding) |
| | | transporting | |
| | | localizing | Nuclear localization sequence, mitochondrial |
| | | | targeting signal, lysosome sorting signal, ER |
| | | | localization signal, peroxisomal targeting signal |
| 3 | Protein | therapeutic | antibody, enzyme, anticoagulant, growth factor, |
| | | | hormone, interferon, interleukin, proteinase |
| | | | inhibitor |
| 3 | | targeting | antibody, nanobody, affibody, affilin, anticalin, |
| | | | atrimer, DARPin, FN3 scaffold, fynomer, |
| | | | kunitz domain, pronectin, OBody, avimer, |
| | | | lectin |
| 4 | | carrier | human serum albumin (HSA), bovine serum |
| | | | albumin (BSA, milk beta-lactoglobulin (β-LG), |
| | | | immunogenic proteins (e.g., diphtheria toxoid, |
| | | | tetanus toxoid), keyhole limpet hemocyanin |
| 5 | Carbohydrate | therapeutic | Anti-inflammatory (e.g., hyaluronic acid), anti- |
| | | | coagulant (e.g., heparin) and antithrombotic |
| | | | agents (e.g., fondaparinux) |
| 6 | | targeting | galactose, galactosamine, N-formyl- |
| | | | galactosamine, N-acetylgalactosamine, N- |
| | | | propionyl-galactosamine, N-n-butanoyl- |
| | | | galactosamine, N-isobutanoylgalactose-amine, |
| | | | lactose, mannose, mannose-6-phosphate, D- |
| | | | rhamnose, heparin, heparan sulfate, sLe^{x} or sLe^{a} |
| 7 | | carrier | chitosan, hyaluronic acid, amylose, dextran, |
| | | | pectin |
| 9 | Polymer | carrier | Polyamines [triethylenetetraamine, spermine, |
| | | | polyspermine, spermidine, synnorspermidine, |
| | | | C-branched spermidin], polymethacrylates |
| | | | (e.g., hydroxylpropyl methacrylate (HPMA)), |
| | | | poly(L-lactide), poly(DL lactide-co-glycolide |
| | | | (PGLA), dendrimers, polyacrylic acids, |
| | | | polyethylenimines (PEI), polyalkylacrylic |
| | | | acids, polyurethanes, polyacrylamides, N- |
| | | | alkylacrylamides, polyspermine (PSP), |
| | | | polyethers, cyclodextrins, derivatives thereof |
| | | | and co-polymers thereof |
| 11 | Hydrophobic or | PK/PD modulation, | C16-20 hydrophobic group, a sterol, |
| | lipophilic group | carrier | cholesterol |
| | | | palmitoyl, hexadec-8-enoyl, oleyl, (9E, |
| | | | 12E)-octadeca-9,12-dienoyl, dioctanoyl, |
| | | | and C16-C20 acyl |
| | | | thiocholesterol, |
| | | | lanosterol, |
| | | | coprostanol, |
| | | | stigmasterol, |
| | | | ergosterol, calciferol, cholic acid, |
| | | | deoxycholic acid, estrone, estradiol, |
| | | | estratriol, progesterone, stilbestrol, |
| | | | testosterone, androsterone, |
| | | | deoxycorticosterone, cortisone, 17- |
| | | | hydroxycorticosterone |
| 12 | vitamin | targeting | folate and derivatives, folate analogues, |
| | | | thiamine, riboflavin, nicotinic acid or |
| | | | niacin, vitamin B6, pantothenic acid, |
| | | | biotin, folic acid, inositol, choline and |
| | | | ascorbic acid, vitamin A, vitamin D |
| 13 | active drug | therapeutic | aspirin, ibuprofen, antidiabetics, antibacterials, |
| | | | antibiotics, antivirals, chemotherapeutics |
| 14 | label | reporting | biotin, fluorescein, coumarin |
| | | | fluorescent/chemiluminescent/bioluminesc |
| | | | ent marker compounds |
| 15 | Other molecules | miscellaneous | flavonoids, chelators, intercalators |

### EXAMPLES

### Example 1—Nomenclature of Molecules Used in this Invention

The nucleic acid nanoparticles used in this invention are interchangeably referred to as Mergo.

RNA Strands Covered in this Invention

TABLE 6
construct strands

| | | Sequence | |
|---|---|---|---|
| | | length | |
| Identifier | Sequence | (RNA) | Modifications/comments |
| C-1.0 | GGGAAAcuc | 47 nt | 2′F U, C (on all pyrimidines) |
| C-1.1 | uGucGuGGG | 47 nt | 2′F U, C (on all pyrimidines), 5′ norbornene modifier |
| C-1.2 | AcGGucAGA | 47 nt | 2′F U, C (on all pyrimidines), full phosphorothioate |
| | cuGuucAAcc | | backbone, 5′ norbornene modifier |
| C-1.3 | Acuccucuuc | 47 nt | 2′F U, C (on all pyrimidines), 2′OMe A, G (on all |
| | | | purines), 5′ norbornene modifier |
| C-1.4 | | 47 nt | Fully 2′OMe modified, 5′ norbornene modifier |
| C-1.5 | | 47 nt | 2′F U, C (on all pyrimidines), conjugated to 2 x PPIB |
| | | | via combinatorial cargo strategy |
| C-1.6 | | 47 nt | 2′F U, C (on all pyrimidines), conjugated to PLK1 |
| | | | via disulfide |
| C-2.0 | GGGAAAGA | 47 nt | 2′F U, C (on all pyrimidines) |
| C-2.1 | AGAGGAGu | 47 nt | 2′F U, C (on all pyrimidines), full phosphorothioate |
| | GGAcGGuAc | | backbone |
| C-2.2 | uGuGuuucAA | 47 nt | 2′F U, C (on all pyrimidines), 2′OMe A, G (on all |
| | ccuGucucuGA | | purines) |
| C-2.3 | c | 47 nt | 2′F U, C (on all pyrimidines), histamine azide clicked |
| | | | onto the 2′ position of A (at 8 locations) |
| C-2.4 | | 47 nt | 2′F U, C (on all pyrimidines), 1-azidododecane |
| | | | clicked onto the 2′ position of A (at 12 locations) |
| C-2.5 | | 47 nt | 2′F U, C (on all pyrimidines), arginine azide clicked |
| | | | onto the 2′ position of A (at 12 locations) |
| C-2.6 | | 47 nt | 2′F U, C (on all pyrimidines), PEG-7 clicked onto the |
| | | | 2′ position of A (at 12 locations) |
| C-2.7 | | 47 nt | 2′F U, C (on all pyrimidines), 5′ TEG cholesterol |
| C-2.8 | | 47 nt | Fully 2′OMe modified |
| C-2.9 | | 47 nt | 2′F U, C (on all pyrimidines), conjugated to peptide |
| | | | with linear sequence GFWFG |
| C-3.0 | GGGAAAGc | 47 nt | 2′F U, C (on all pyrimidines) |
| C-3.1 | AGuGuAGcG | 47 nt | 2′F U, C (on all pyrimidines), full phosphorothioate |
| | GAcGGuGuG | | backbone |
| C-3.2 | ucAGuucAAc | 47 nt | 2′F U, C (on all pyrimidines), 2′OMe A, G (on all |
| | ccAcGAcAG | | purines) |
| C-3.3 | AG | 47 nt | 2′F U, C (on all pyrimidines), histamine azide clicked |
| | | | onto the 2′ position of A (at 8 locations) |
| C-3.4 | | 47 nt | 2′F U, C (on all pyrimidines), PEG-7 clicked onto the |
| | | | 2′ position of A (at 12 locations) |
| C-3.5 | | 47 nt | 2′F U, C (on all pyrimidines), arginine azide clicked |
| | | | onto the 2′ position of A (at 12 locations) |
| C-3.6 | | 47 nt | Fully 2′OMe modified |
| C-3.7 | | 67 nt | 2′F U, C (on all pyrimidines), extended region for |
| | | | hybridization of aptamer sequence |
| C-4.0 | GGGAAAGu | 47 nt | 2′F U, C (on all pyrimidines) |
| C-4.1 | CAGAGACAG | 47 nt | 2′F U, C (on all pyrimidines), 5′ norbornene modifier |
| C-4.2 | GAcGGucuA | 47 nt | 2′F U, C (on all pyrimidines), full phosphorothioate |
| | GGucuucAAc | | backbone, 5′ norbornene modifier |
| C-4.3 | cGcuAcAcuG | 47 nt | 2′F U, C (on all pyrimidines), 2′OMe A, G (on all |
| | c | | purines), 5′ norbornene modifier |
| C-4.4 | | 47 nt | Fully 2′OMe modified, 5′ norbornene modifier |
| C-4.5 | | 47 nt | 2′F U, C (on all pyrimidines), conjugated to 2 x PPIB |
| | | | via combinatorial cargo strategy |
| C-4.6 | | 47 nt | 2′F U, C (on all pyrimidines), conjugated to PLK1 |
| | | | via disulfide |
| C-5.0 | GGGAAAcu | 58 nt | 2′F U, C (on all pyrimidines) |
| C-5.1 | AGAuuGGA | 58 nt | 2′F U, C (on all pyrimidines), 5′ Cy3 |
| C-5.2 | AcAcAGuAu | 58 nt | 2′F U, C (on all pyrimidines), 5′ Cy3, 1- |
| | uGGAcAGuc | | azidododecane clicked onto the 2′ position of A (at 8 |
| | uGAuuGGAc | | locations) |
| C-5.3 | uGAcAcAuu | 58 nt | 2′F U, C (on all pyrimidines), 5′ Cy3, full |
| | GGAGAc | | phosphorothioate backbone |
| C-5.4 | | 58 nt | 2′F U, C (on all pyrimidines), 5′ Cy3, 1- |
| | | | azidododecane clicked onto the 2′ position of A (at 4 |
| | | | locations) |
| C-5.5 | | 58 nt | 2′F U, C (on all pyrimidines), 2′OMe A, G (on all |
| | | | purines), 5′ Cy3 |
| C-5.6 | | 58 nt | 2′F U, C (on all pyrimidines), 5′ Cy3 |
| C-5.7 | | 58 nt | 2′F U, C (on all pyrimidines), PEG-7 clicked onto the |
| | | | 2′ position of A (at 19 locations), 5′ Cy3 |
| C-5.8 | | 58 nt | 2′F U, C (on all pyrimidines), 3′ TEG cholesterol, 5′ |
| | | | Cy3 |
| C-5.9 | | 58 nt | Fully 2′OMe modified, 5′ Cy3 |
| C-6.0 | AccuGAccAu | 13 nt | 2′F U, C (on all pyrimidines) |
| | Guc | | |
| C-7.0 | AcuGAucGu | 28 nt | 2′F U, C (on all pyrimidines) |
| | AGcAcuGuAc | | |
| | AAGucAccG | | |
| C-8.0 | cGGuGAcuu | 13 nt | 2′F U, C (on all pyrimidines) |
| | GuAc | | |
| C-9.0 | AGuGcuAcG | 28 nt | 2′F U, C (on all pyrimidines) |
| | AucAGuGAc | | |
| | AuGGucAGG | | |
| | u | | |

TABLE 7
siRNA strands

| Identifier | Sequence | Modifications/comments |
|---|---|---|
| S-1.0 | CAA AUU CCA UCG UGA | Ppib sense strand unmodified |
| S-1.1 | c*mA*amAumUcmCamUcmGu*m | Ppib sense strand, 2′ F C/A/U indicated |
| | G*a | with lowercase, 2′ O me A/U/G/C |
| | | indicated with ′m′, PTO indicated with* |
| S-1.2 | 5′ amine | Ppib sense strand, 2′ F C/A/U indicated |
| | c*mA*amAumUcmCamUcmGu*m | with lowercase, 2′ O me A/U/G/C |
| | G*a | indicated with ′m′, PTO indicated with*, 5′ |
| | | amino modifier |
| S-1.3 | 5′ tetrazine | Ppib sense strand, 2′ F C/A/U indicated |
| | c*mA*amAumUcmCamUcmGu*m | with lowercase, 2′ O me A/U/G/C |
| | G*a | indicated with ′m′, PTO indicated with*, 5′ |
| | | tetrazine |
| S-1.4 | Cy3- | Ppib sense strand, 2′ F C/A/U indicated |
| | c*mA*amAumUcmCamUcmGu*m | with lowercase, 2′ O me A/U/G/C |
| | G*a | indicated with ′m′, PTO indicated with*, 5′ |
| | | Cy3 |
| S-2.0 | UCA CGA UGG AAU UUG CUG | Ppib antisense strand unmodified |
| | UU | |
| S-2.1 | mU*c*mAcmGamUgmGamAumU | Ppib antisense strand, 2′ F C/A/U/G |
| | *u*mG*c*mU*g*mU*u | indicated with lowercase, 2′ O me A/U/G |
| | | indicated with ′m′, PTO indicated with * |
| S-3.0 | GcAAuuAcAuGAGcGAGcATT | Sense strand, PLK1-targeting canonical |
| | | siRNA, 2′F U, C |
| S-3.1 | GCAAUUACAUGAGCGAGCAT | Sense strand, PLK1-targeting canonical |
| | T | siRNA |
| S-4.0 | uGcucGcucAuGuAAuuGcGG | Antisense strand, PLK-1 targeting |
| | | canonical siRNA, 2′F U, C |
| S-4.1 | UGCUCGCUCAUGUAAUUGCG | Antisense strand, PLK1-targeting |
| | G | canonical siRNA |

TABLE 8
siRNA duplexes

| Identifier | siRNA strands used | Conformation |
|---|---|---|
| si-1.0 | S-1.4, S-2.1 | Simple duplex |
| si-2.0 | S-1.1, S1.4, S-2.1 | Two siRNA molecules linked |
| | | together via a poly-T region |
| si-3.0 | S-3.0, S-4.0 | Simple duplex |
| si-4.0 | S-3.1, S-4.1 | Simple duplex |

TABLE 9
RNA constructs

| Identifier | Strands used | Modifications |
|---|---|---|
| M-1 | C-1.0, C-2.0, C-3.0, | 2′F U, C (on all pyrimidines) |
| | C-4.0, C-5.0 | |
| M-2 | C-1.0, C-2.0, C-3.0, | 2′F U, C (on all pyrimidines), 5′ |
| | C-4.0, C-5.1 | Cy3 |
| M-3 | C-1.1, C-2.0, C-3.0, | 2′F U, C (on all pyrimidines), 5′ |
| | C-4.1, C-5.2, S-1.3, | Cy3, 1-azidododecane clicked onto |
| | S-2.1 | the 2′ position of A (at 8 locations |
| | | on C-5.2), 2 × PPIB siRNA |
| | | conjugated via IEDDA |
| M-4 | C-1.1, C-2.0, C-3.0, | 2′F U, C (on all pyrimidines), 5′ |
| | C-4.1, C-5.3, S-1.3, | Cy3, full phosphorothioate |
| | S-2.1 | backbone on one strand (C-5.3), 2 × |
| | | PPIB siRNA conjugated via |
| | | IEDDA |
| M-5 | C-1.1, C-2.0, C-3.0, | 2′F U, C (on all pyrimidines), 5′ |
| | C-4.1, C-5.4, S-1.3, | Cy3, 1-azidododecane clicked onto |
| | S-2.1 | the 2′ position of A (at 4 locations |
| | | on C-5.4), 2 × PPIB siRNA |
| | | conjugated via IEDDA |
| M-6 | C-1.1, C-2.1, C-3.1, | 2′F U, C (on all pyrimidines), full |
| | C-4.1, C-5.3, S-1.3, | phosphorothioate backbone (C-2.1, |
| | S-2.1 | C-3.1, C-5.3), 5′ Cy3, 2 × PPIB |
| | | siRNA conjugated via IEDDA |
| M-7 | C-1.1, C-2.2, C-3.2, | 2′F U, C (on all pyrimidines), |
| | C-4.1,C-5.5, S-1.3, | 2′OMe A, G (on all purines) (on C- |
| | S-2.1 | 2.2, C-3.2, C-5.5), 5′ Cy3, 2 × PPIB |
| | | siRNA conjugated via IEDDA |
| M-8 | C-1.1, C-2.3, C-3.3, | 2′F U, C (on all pyrimidines), |
| | C-4.1, C-5.1, S-1.3, | histamine azide clicked onto the 2′ |
| | S-2.1 | position of A (at 8 locations on C- |
| | | 2.3, C-3.3), 5′ Cy3, 2 × PPIB |
| | | siRNA conjugated via IEDDA |
| M-9 | C-1.1, C-2.6, C-3.4, | 2′F U, C (on all pyrimidines), PEG- |
| | C-4.1, C-5.7, S-1.3, | 7 clicked onto the 2′ position of A |
| | S-2.1 | (C-2.6, C-3.4, C-5.7), 5′ Cy3, 2 × |
| | | PPIB siRNA conjugated via |
| | | IEDDA |
| M-10 | C-1.2, C-2.1, C-3.1, | 2′F U, C (on all pyrimidines), 5′ |
| | C-4.2, C-5.3, S-1.3, | Cy3, full phosphorothioate |
| | S-2.1 | backbone on all strands, 2 × PPIB |
| | | siRNA conjugated via IEDDA |
| M-11 | C-1.4, C-2.8, C-3.6, | Fully 2′OMe modified, 5′ Cy3, 2 × |
| | C-4.4, C-5.9, S-1.3, | PPIB siRNA conjugated via |
| | S-2.1 | IEDDA |
| M-12 | C-1.1, C-2.5, C-3.5, | 2′F U, C (on all pyrimidines), |
| | C-4.1, C-5.1, S-1.3, | arginine azide clicked onto the 2′ |
| | S-2.1 | position of A (C-2.5, C-3.5), 5′ |
| | | Cy3, 2 × PPIB siRNA conjugated |
| | | via IEDDA |
| M-13 | C-1.1, C-2.7, C-3.0, | 2′F U, C (on all pyrimidines), 3′ |
| | C-4.1, C-5.8, S-1.3, | TEG cholesterol, 5′ Cy3, 2 × PPIB |
| | S-2.1 | siRNA conjugated via IEDDA |
| M-14 | C-1.1, C-2.0, C-3.0, | 2′F U, C (on all pyrimidines), 5′ |
| | C-4.1, C-5.1, S-1.3, | Cy3, 2 × PPIB siRNA conjugated |
| | S-2.1 | via IEDDA |
| M-15 | C-1.1, C-2.0, C-3.0, | 2′F U, C (on all pyrimidines), 5′ |
| | C-4.0, C-5.1, S-1.3, | Cy3, 1 × PPIB siRNA conjugated |
| | S-2.1 | via IEDDA |
| M-16 | C-1.5, C-2.0, C-3.0, | 2′F U, C (on all pyrimidines), 5′ |
| | C-4.0, C-5.1, S-1.3, | Cy3, 4 × PPIB siRNA conjugated |
| | S-2.1 | via IEDDA |
| M-17 | C-1.5, C-2.0, C-3.0, | 2′F U, C (on all pyrimidines), 5′ |
| | C-4.5, C-5.1, S-1.3, | Cy3, 8 × PPIB siRNA conjugated |
| | S-2.1 | via IEDDA |
| M-18 | C-1.6, C-2.9, C-3.7, | 2′F U, C (on all pyrimidines), EE |
| | C-4.6, C-5.1, S-3.0, | peptide, aptamer sequence |
| | S-4.0 | (hybridized to C-3.7), 5′ Cy3 |
| M-19 | C-1.1, C-2.0, C-3.0, | 2′F U, C (on all pyrimidines), 5′ |
| | C-4.1, C-5.3, S-1.3, | Cy3, full phosphorothioate |
| | S-2.1 | backbone, 2 × PPIB siRNA |
| | | conjugated via IEDDA |
| M-20 | C-1.0, C-2.1, C-3.1, | 2′F U, C (on all pyrimidines), 5′ |
| | C-4.0, C-5.3, S-1.3, | Cy3, full phosphorothioate |
| | S-2.1 | backbone (C-2.1, C-3.1, C-5.3), 2 × |
| | | PPIB siRNA conjugated via |
| | | IEDDA |
| M-21 | C-6.0, C-7.0, C-8.0, | 2′F U, C (on all pyrimidines) |
| | C-9.0 | |
| M-22 | Compact form of M-1 | 2′F U, C (on all pyrimidines) |
| | (and analogues). | |
| | 200 nt total. | |

Note:
where norbornene/tetrazine-labelled strands are shown, these were conjugated prior to assembly to form the IEDDA product(s).

### Example 2—RNA Synthesis

Oligonucleotides were synthesized on 1-10 μmol scale using a K&A synthesizer (H-16). All protocols were modified depending on the sequence requirements. Phosphoramidites and CPGs with standard protecting groups were purchased from ChemGenes and Glen Research. Adenosine phosphoramidites containing amino acids, amino acid analogues, PEGs and hydrocarbon chains were synthesized in-house. The detritylation step was carried out with 3% TCA in DCM, followed by coupling with 0.1M phosphoramidite solutions and 0.25M BMT in MeCN. Capping was performed using THF/lutidine/acetic anhydride (80/10/10) as capping A and 16% N-methylimidazole in THE as capping B, respectively. The oxidation step was accomplished with 0.02 M iodine solution in THF/Pyr/water (90.6/0.4/9).

All synthesized oligonucleotides were cleaved and deprotected using aq. methylamine/ammonium hydroxide solution (1:1) for 3 h at RT for a solid support with a first base attached or for 1 h at 65° C. for a universal CPG. The removal of tert-butyl silyl protecting groups was performed by incubating an intermediate product in DMSO Et₃N·3HF for 3 h at 65° C. Crude oligonucleotides were subsequently precipitated from ethanolic solution containing sodium acetate. After 2 h at −70° C. the precipitate was harvested by 25 min centrifugation at 4° C. (14,000 rpm). The supernatant was separated, and the remaining pellet was washed repeatedly with 70% EtOH. After a final wash, the crude sample was dried under vacuum in a speedvac and redissolved in water for purification.

Crude RNA strands were purified either by IEX-HPLC or by IP-RP HPLC.

IEX was carried out with a preparative DNAPac PA200 (ThermoFisher), 22×250 mm column, or PL-SAX (Agilent) 22×150 mm 1000 Å column at 75° C. with a flow rate of 15 mL/min and UV detection at 260 nm. Elution was performed with a linear gradient selected based on crude impurity profile, determined by analytical testing using either a DNAPac PA200RS UPLC column or PL-SAX analytical column. Buffer A: 25 mM Tris HCl, pH 8.0, 20% acetonitrile, 10 mM sodium perchlorate; buffer B: 25 mM Tris HCl, pH 8.0, 20% acetonitrile, 600 mM sodium perchlorate, OR, Buffer A: 25 mM Tris HCl, pH 8.0, 20% acetonitrile, 25 mM sodium chloride; buffer B: 25 mM Tris HCl, pH 8.0, 20% acetonitrile, 1.5M sodium chloride.

RP-HPLC was carried out with a BEH C18 300 Å (Waters) 19×150 mm at 60° C., with a flow rate of 25 mL/min and UV detection at 260 nm. Buffer A: TEAA (0.1 M, pH=7); buffer B: MeCN, OR, Buffer A: HAA (0.1M, pH7); buffer B: MeCN.

Fractions containing RNA were assessed for purity by analytical PAGE, IEX and RP-HPLC, then pooled and subject to final QC on PAGE, IEX and RP-HPLC, acetonitrile removed in vacuo. The purified oligos were then desalted with Gel-Pak desalting columns (Glen). The solution was lyophilized, and the RNA dissolved in nuclease-free water for concentration determination by UV absorbance and quality assessment via denaturing PAGE.

### Adaptation to Synthesis Procedure for Modified Strands

5′ amino—10% DEA solution in MeCN was applied onto the oligonucleotide while still on CPG. After 5 min treatment the column was rinsed with MeCN and processed further.

5′ Cy3—MMTr group at 5′-end of Cy3 containing sequences was removed during RPC MMT-ON purification.

5′ cholesterol modification—10% DEA solution in MeCN was applied onto the oligonucleotide while still on CPG. After 5 min treatment the column was rinsed with MeCN, and the protecting group (DMT) was removed while still on solid support prior to cleavage and deprotection steps.

### Example 3—Optimized Assembly Protocol

The key scaffold in this work was assembled according to a standard protocol. Equimolar amounts of the 5 different strands, C-1.0, C-2.0, C-3.0, C-4.0 and C-5.0 (and sub-variants in Table 6) were combined in PBS+MgCl₂ (2 mM) buffer, with a final concentration of 10 μM. The 5 strands were annealed to each other at 95° C. for 5 min then slowly cooled down to 15° C. The scaffold was then analyzed by native polyacrylamide gel electrophoresis (PAGE) and dynamic light scattering (DLS) (vide infra).

For PAGE, the assembled scaffold was electrophoresed on native PAGE (6%) in 1×TBMg (890 mM Tris Borate+20 mM Mg(OAc)₂, pH=8.3) at a constant voltage of 100 V. Gel bands were visualized using GelRed™. 10 pmol of structures was loaded. 2 μL of glycerin (70% in H₂O) was added to samples before loading.

For DLS, the assembled scaffold was analyzed using a Malvern Zetasizer Nano S ZEN 1600 Nano Particle Size Analysis—20 μL of samples were used, and intensity was recorded. Average of three trials was calculated. All measurements were carried out at 25° C. Samples were centrifuged at 12000 rpm for 5 minutes before analysis to remove dust and debris.

### Further Optimization to Reduce Overall RNA Content of the Nucleic Acid Nanoconstructs

The key scaffold used in this invention has been further refined to reduce the overall RNA content by 19%, which allows for more cost-effective manufacturing. The optimized characteristics are given in FIG. 62. This nanoconstruct retains all the functionality of the original constructs but can be obtained in greater yields and with less by-products, due to the removal of unhybridized sections.

### Example 4

Solid phase oligonucleotide synthesis of core strand conjugated to siRNA using C-6 disulfide modifier.

Synthesis: The sequence containing disulfide linkage was synthesized using the following reagents: 3% TCA in DCM, 0.25M Hyacinth BMT solution, CAP A (THF/lutidine/acetic anhydride), CAP B (16% N-methylimidazole), 0.02M Iodine/Py/water.

Phosphoramidites: 2′-tBDSilyl Adenosine (n-bz) CED phosphoramidite, 2′-tBDSilyl Cytidine (n-acetyl) CED phosphoramidite, 2′-tBDSilyl Guanosine (n-ibu) CED phosphoramidite, 2′-tBDSilyl Uridine CED phosphoramidite, 2′-Fluoro-2′-deoxyCytidine (n-ac) CED phosphoramidite, 2′-Fluoro-2′-deoxy Uridine CED phosphoramidite, Thymidine CED phosphoramidite

5′-thiol modifier C6: formula (VIII):

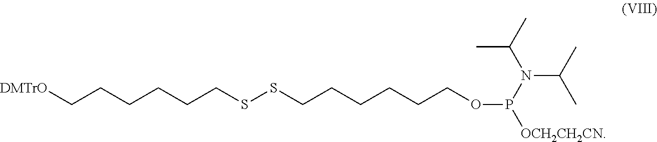

Deprotection: AMA, rt, 3 h. TEA×3HF, 65° C.

Quality control of a raw material is provided in FIG. 63:

Purification method: The sequence was purified using IEX chromatography, using DNAPac_PA100 22×250 mm at 75° C.

| | |
|---|---|
| Buffer A | 25 mM Tris•HCl, pH 8.0, 20% acetonitrile, 10 mM sodium |
| | perchlorate |
| Buffer B | 25 mM Tris•HCl, pH 8.0, 20% acetonitrile, 600 mM sodium |
| | perchlorate |
| pH | 8 |
| Gradient | Buffer B 0 to 25% in 5 min; 25% to 40% in 30 min |

Isolation yield: 25%.

### Example 5—IEDDA and SPAAC Conjugation of siRNA to RNA

To install the appropriate reactive groups to enable conjugation chemistry, 5′ amino modified RNA strands were treated with heterobifunctional NHS-linkers containing the same.

### General Coupling Procedure

The amino-modified oligonucleotide was prepared as a stock solution or dry aliquot. The heterobifunctional NHS-ester (NHS-SM) was dissolved at a concentration of 100 mM in anhydrous DMSO.

Amino-modified oligonucleotide was diluted to a final concentration of 100-200 μM, followed by the addition of DMSO (50% total volume), bicarbonate buffer (0.5 M, pH=8.4, 20% total volume) and NHS-SM (5-20 eq). The reaction mixture was agitated at 30° C. for 1-3 h and was then purified by RP-HPLC. With higher volumes, EtOH precipitation and resuspension in H₂O is recommended.

### Modified Coupling Procedure for Tetrazine NHS

The amino-modified oligonucleotide was prepared as a stock solution or dry aliquot. The heterobifunctional NHS-ester (NHS-SM) was dissolved at a concentration of 100 mM in anhydrous DMF.

Amino-modified oligonucleotide was diluted to a final concentration of 200-500 μM, followed by the addition of DMF (35% total volume), sodium chloride/bicarbonate buffer (100 mM NaCl, 0.05 M, pH=8.4, 30% total volume) and NHS-Tetrazine (5-20 eq). The reaction mixture was agitated at 30° C. for 1 h and was then purified by EtOH precipitation and resuspension in H₂O.

### Example of IEDDA Click Procedure

Norbornene modified core strand C-4.4 (5 nmol, 1.0 eq, 1400 μM final concentration) was mixed with siRNA functionalized via tetrazine-NHS (5-1.5, 15 nmol, 1.6 eq) in PBS buffer. The reaction mixture was agitated at RT for 12 h, followed by purification with IEX chromatography, using DNAPac_PA100 22×250 mm column at 75° C., at a flow rate of 25 mL/min. 40% to 60% B in 30 min (A: 0.1 M NaCl pH 7, B: 1.0M NaCl), fractions containing product were concentrated and desalted, resulting in 44% isolated yield.

### Example 6—General Small Molecule Synthetic Procedures

### General Experimental

¹H NMR spectra were recorded at 400 MHz. ¹³C NMR spectra were recorded at 100 MHz. Chemical shifts (δ) are quoted in units of parts per million (ppm) downfield from tetramethylsilane and are referenced to a residual solvent peak. (CDCl₃ (δ_{H}: 7.26, δ_{C}: 77.0)). Coupling constants (J) are quoted in units of Hertz (Hz). The following abbreviations are used within ¹H NMR analysis: s=singlet, d=doublet, t=triplet, q=quartet, pent=pentet, m=multiplet, dd=doublet of doublets, dt=doublet of triplets. Spectra recorded at 400 (¹H NMR) and 100 (¹³C NMR) were carried out by the Imperial College London Department of Chemistry NMR Service.

Low- and high-resolution mass spectrometry (EI, CI, FAB) were recorded at Imperial College London. Measurements carried out by the Imperial College Department of Chemistry Mass Spectrometry Service used a Micromass Platform II and Micromass AutoSpec-Q spectrometer.

Flash column chromatography was carried out on BDH silica gel 60, particle size 0.040-0.063 mm. Thin layer chromatography (TLC) was performed on pre-coated aluminum backed or glass backed plates (Merck Kieselgel 60 F254), and visualized with ultraviolet light (254 nm) or potassium permanganate (KMnO₄), vanillin or phosphomolybdic acid (PMA) stains.

### Example 7—Synthesis of Highly Reactive Labile Compounds

### 6,6′-Disulfanediylbis(hexan-1-ol)

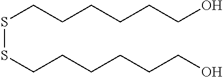 [SMILES: OCCCCCCSSCCCCCCO]

Synthesized according to a procedure outlined by Varenikov and co-workers (A. Varenikov, M. Gandelman, Organotitanium Nucleophiles in Asymmetric Cross-Coupling Reaction: Stereoconvergent Synthesis of Chiral α-CF 3 Thioethers, J. Am. Chem. Soc. 141 (2019) 10994-10999). Colorless oil obtained (18.2 g, 92%). ¹H NMR (400 MHz, Chloroform-d) δ 3.65 (t, J=6.6 Hz, 4H), 2.70 (dd, J=7.9, 6.8 Hz, 4H), 1.81-1.77 (m, 2H), 1.76-1.65 (m, 4H), 1.59 (dq, J=7.9, 6.6 Hz, 4H), 1.51-1.32 (m, 8H).

### 6-((6-Hydroxyhexyl)disulfaneyl)hexyl bicyclo[2.2.1]hept-5-ene-2-carboxylate

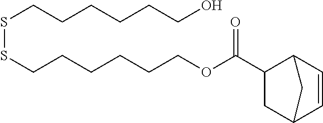 [SMILES: O=C(OCCCCCCSSCCCCCCO)C1CC2C=CC1C2]

Synthesized according to a modified procedure found in the art (US2011/263526). A solution of DCC (1.15 g in 5 mL anhydrous DCM, 5.61 mmol) was added dropwise to a stirred solution of 5-norbornene-2-carboxylic acid (500 mg, 3.62 mmol), 6,6′-disulfanediylbis(hexan-1-ol) (1.93 g, 7.25 mmol) and DMAP (89 mg, 0.72 mmol) in anhydrous DCM (20 mL) over 5 min at 0° C. The reaction mixture was then stirred at 0° C. for 3 h. Upon completion (TLC: 25% EtOAc/pentane), the reaction mixture was filtered. The filtrate was then washed with water (3×20 mL) and brine (3×20 mL). The organic layer was then dried (MgSO₄) and concentrated in vacuo. The crude residue was then purified by column chromatography (20 to 30% EtOAc/pentane), affording the title compound as a colorless oil (469 mg, 34%). ¹H NMR (400 MHz, Chloroform-d) δ 6.22 (dd, J=5.7, 3.1 Hz, 1H), 5.94 (dd, J=5.7, 2.9 Hz, 1H), 4.04 (td, J=6.6, 4.2 Hz, 2H), 3.23 (dq, J=3.4, 1.8 Hz, 1H), 2.95 (ddd, J=12.6, 4.7, 3.0 Hz, 2H), 2.71 (td, J=7.3, 2.2 Hz, 5H), 1.93 (ddd, J=12.6, 9.3, 3.7 Hz, 1H), 1.72 (dt, J=7.2, 4.0 Hz, 4H), 1.51-1.37 (m, 14H), 1.36-1.26 (m, 1H); HRMS ES+ (m/z): [M]⁺ calc'd for C₂₀H₃₄O₃: 386.6090; found: 386.6097.

### 6-((6-(((2-cyanoethoxy)(diisopropylamino)phosphaneyl)oxy)hexyl)disulfaneyl)hexyl bicyclo[2.2.1]hept-5-ene-2-carboxylate

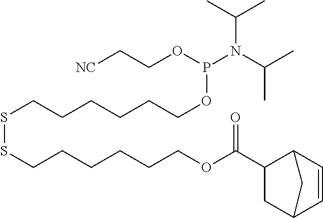 [SMILES: CC(C)N(C(C)C)P(OCCC#N)OCCCCCCSSCCCCCCOC(=O)C1CC2C=CC1C2]

6-((6-Hydroxyhexyl)disulfaneyl)hexyl bicyclo[2.2.1]hept-5-ene-2-carboxylate (496 mg, 0.87 mmol) and N,N-diisopropylethylamine (451 mg, 609 μL, 3.49 mmol) were dissolved in anhydrous DCM (15 mL) and stirred over activated molecular sieves for 1 h at 0° C. 2-Cyanoethyl N,N-diisopropylchlorophosphoramidite (413 mg, 1.74 mmol) was added and the reaction mixture was stirred for 30 min at 0° C., and was then slowly warmed to RT over 1.5 h. Upon completion (TLC: 25% EtOAc/pentane), the reaction mixture was washed with sat. NaHCO₃ (3×20 mL). The organic layer was then dried (MgSO₄) and concentrated in vacuo, and the crude product was purified by column chromatography (10% EtOAc/pentane+1% Et₃N), affording the title compound was a colorless oil (341 mg, 67%). ¹H NMR (400 MHz, Chloroform-d) δ 6.21 (dd, J=5.7, 3.1 Hz, 1H), 5.94 (dd, J=5.7, 2.9 Hz, 1H), 4.04 (td, J=6.6, 4.0 Hz, 2H), 3.92-3.78 (m, 2H), 3.75-3.54 (m, 4H), 3.23 (dd, J=4.1, 2.3 Hz, 1H), 3.03-2.88 (m, 2H), 2.73-2.67 (m, 6H), 2.07 (s, 1H), 1.92 (ddd, J=11.8, 9.3, 3.7 Hz, 1H), 1.70 (d, J=7.2 Hz, 3H), 1.66-1.61 (m, 4H), 1.45-1.39 (m, 8H), 1.30 (t, J=4.4 Hz, 1H), 1.21 (dd, J=6.8, 4.1 Hz, 14H); ³¹P NMR (162 MHz, Chloroform-d) δ 147.26; HRMS ES+ (m/z): [M]⁺ calc'd for C₂₉H₅₁O₄PS₂: 586.3028; found: 586.8304.

This compound may be incorporated into any designs shown in FIG. 1-6.

### Example 8—Synthesis of Compounds to Modulate PK/PD Activity

### General Procedure A—CuAAC of Azide Modifications for 2′O Functionalization

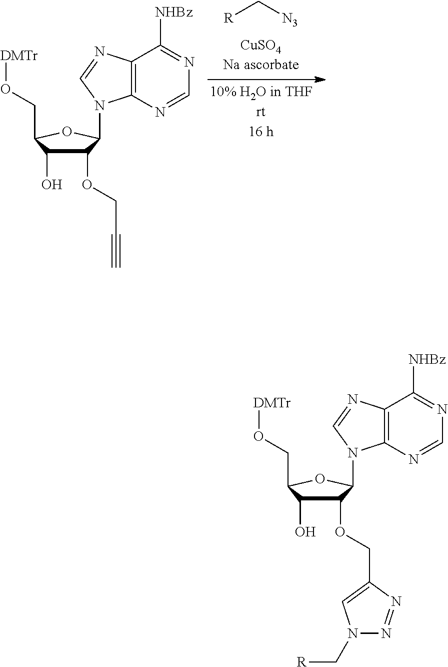

N-(9-((2R,3R,4R,5R)-5-((bis(4-methoxyphenyl)(phenyl)methoxy)methyl)-4-hydroxy-3-(prop-2-yn-1-yloxy)tetrahydrofuran-2-yl)-9H-purin-6-yl)benzamide (2′O propargyl A) (1 equiv.) and the azide (R—N₃) (1.2 equiv.) were dissolved in THE (0.1 M reaction concentration). Copper(II) sulfate (0.085 equiv.) and sodium ascorbate (0.1 equiv.) were added under N₂ and the mixture was stirred for 16 h. Upon completion, a 5% solution of EDTA was added and the reaction mixture was extracted with ethyl acetate. The organic layer was dried (MgSO₄) and concentrated in vacuo. The crude residue was then purified by column chromatography to afford the title compound.

PK/PD modulating nucleosides, whereby R imparts the biological activity and affects the biodistribution, are given in FIG. 58, FIG. 59. Azides syntheses not outlined in this invention are either commercially available or literature-known.

### General Procedure B—Phosphitylation of 2′O Modified Nucleosides

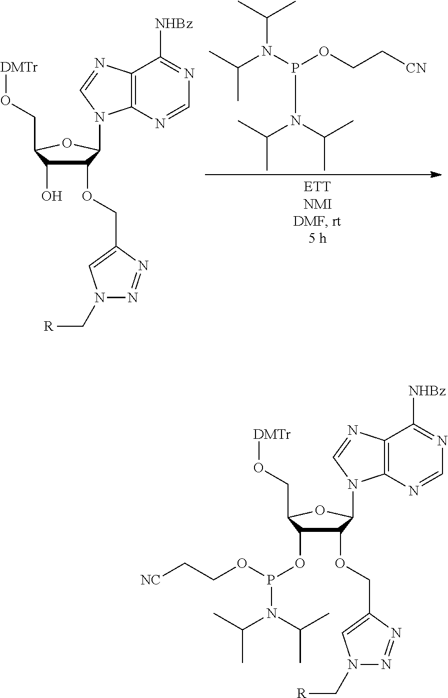

Following a procedure outlined in the art (B. Ross, Q. Song, Process of Purifying Phosphoramidites, U.S. Pat. No. 7,030,230 B2, 2006.), the nucleoside (1 equiv.) was dissolved in anhydrous DMF. Activated molecular sieves were added and the suspension was stirred for 30 min, followed by the addition of 3-((bis(diisopropylamino)phosphaneyl)oxy)propanenitrile (1.5 equiv.) and 5-(ethylthio)-1H-tetrazole (0.82 equiv.). When dissolved, 1-methyl-1H-imidazole (8.3 equiv.) was added, and the reaction mixture was stirred at RT for 5 h.

The following work-up was specifically for 150 mL of DMF as the reaction solvent. Care should be taken to adjust the volumes as appropriate.

When complete, triethylamine (15 mL) was added, and the reaction mixture was diluted with DMF (50 mL). Water (25 mL) was added, and the mixture was extracted with hexane (3×150 mL). The aqueous layer was separated and was diluted with water (75 mL) and was then extracted with toluene (3×225 mL). The upper organic layer was then separated and was washed with DMF:water (60:40 v/v, 3×225 mL) and water (3×150 mL). The upper layer was dried (MgSO₄), filtered and concentrated in vacuo, affording the phosphoramidite, which was often pure enough for direct use.

### 4-(2-Azidoethyl)-1H-imidazole

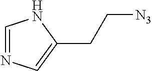

In a mixture of H₂O (48 mL) and DCM (81 mL), NaN₃ (19.9 g, 300 mmol) was dissolved. This solution was cooled on ice. Triflic anhydride (16.9 g, 10.1 mL 60 mmol) was slowly added. This reaction mixture was stirred vigorously for 2 h at room temperature. The water-layer was extracted with DCM (2×50 mL). These combined DCM-layers were washed with saturated aq. Na₂CO₃-solution. Histamine (5.52 g, 184 mmol), K₂CO₃ (16.5 g, 120 mmol) and CuSO₄·5H₂O (47.8 mg, 0.3 mmol) were dissolved in H₂O (96 mL) and MeOH (192 mL). If the amine salt is used instead of the free amine, 1 additional equivalent of K₂CO₃ was added per acidic proton. The freshly made triflic azide in DCM was added. The reaction was stirred overnight at room temperature. The mixture was extracted with DCM (3×200 mL) and water (50 mL). The DCM-layers were combined, dried over Na₂SO₄, filtered, and concentrated in vacuo to give crude azide. A slightly yellow oil was obtained (4.12 g, 87%). The product was pure enough for further synthetic manipulations. An analytical sample was obtained by column chromatography (5% MeOH/DCM) in the dark. This afforded the product as a colorless oil. ¹H NMR (400 MHz, Chloroform-d) δ 8.39 (br s, 1H), 7.66 (br s, 1H), 6.95 (br s, 1H), 3.58 (d, J=6.7 Hz, 2H), 2.90 (t, J=6.7 Hz, 2H). LRMS (ESI+) m/z (%): 138.1 [M]+ (100), 139.1 [M+H]+ (10).

Alternatively, following a protocol described in the art (E. D. Goddard-Borger, R. V. Stick, An Efficient, Inexpensive, and Shelf-Stable Diazotransfer Reagent: Imidazole-1-sulfonyl Azide Hydrochloride, Org. Lett. 13 (2011) 2514-2514), imidazole-1-sulfonyl azide hydrochloride (377 mg, 1.8 mmol) was added to a stirred suspension of histamine (170 mg, 1.5 mmol), K₂CO₃ (414 mg, 3 mmol) and CuSO₄·5H₂O (3.75 mg, 15 μmol) in MeOH (7.5 mL). Upon completion of the reaction, water (5 mL) was added, and the reaction mixture was extracted with DCM (3×5 mL). The organic layer was then dried (MgSO₄) and concentrated, affording the crude title compound as a brown oil (360 mg, 95%), which was used without further purification. ¹H NMR (400 MHz, Chloroform-d) δ 7.64 (s, 1H), 6.92 (s, 1H), 3.61 (t, J=6.7 Hz, 2H), 2.97-2.88 (m, 2H); LRMS ES+ (m/z): [M]⁺ calc'd for C₅H₇N₅; 137.1 found: 138.1 [M+H]⁺.

### (5-(2-Azidoethyl)-1H-imidazol-1-yl)methyl pivalate

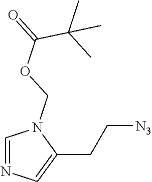 [SMILES: CC(C)(C)C(=O)OCn1cncc1CCN=[N+]=[N-]]

NaH (60% in mineral oil) (41 mg, 1 mmol) was added to THE (4 mL). A solution of 4-(2-azidoethyl)-1H-imidazole (93 mg, 0.68 mmol) in 2 mL THE was added and the resultant suspension was stirred for 3 h at RT. A solution of chloromethyl pivalate (150 mg, 1 mmol) in THE (2 mL) was then added. After 1 h at RT, H₂O (0.5 mL) was added, and the reaction mixture was concentrated in vacuo. The crude residue was redissolved in EtOAc, and the organic layer was washed with water (3×10 mL), brine (3×10 mL), dried (MgSO₄) and concentrated in vacuo. The crude residue was purified by column chromatography (50% EtOAc in pentane) to afford the title compound as a colorless oil (150 mg, 88%). ¹H NMR (400 MHz, Chloroform-d) δ 7.63 (d, J=1.4 Hz, 1H), 6.93 (s, 1H), 5.80 (s, 2H), 3.57 (t, J=6.8 Hz, 2H), 2.85 (td, J=6.8, 0.8 Hz, 2H), 1.18 (s, 9H); ¹³C NMR (101 MHz, Chloroform-d) δ 177.7, 139.6, 138.0, 116.8, 67.6, 50.6, 38.7, 28.1, 26.8. LRMS ES+ (m/z): [M]⁺ calc'd for C₁₁H₁₇N₅O₂; 251.3 found: 252.3 [M+H]⁺

### (5-(2-(4-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methyl)-1H-1,2,3-triazol-1-yl)ethyl)-1H-imidazol-1-yl)methyl pivalate

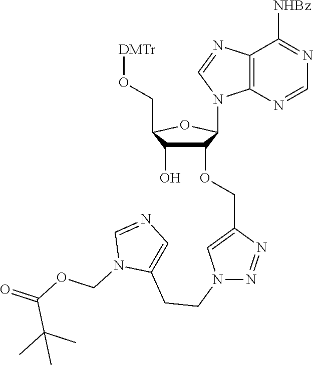

Following general procedure A, the title compound was afforded as a colorless foam (4.53 g, 94%) after column chromatography (5% MeOH/DCM). ¹H NMR (400 MHz, Chloroform-d) δ 8.71 (s, 1H), 8.19 (s, 1H), 8.07-7.98 (m, 2H), 7.62-7.47 (m, 4H), 7.43-7.38 (m, 2H), 7.33-7.16 (m, 9H), 6.84-6.77 (m, 4H), 6.21 (d, J=5.0 Hz, 1H), 5.67 (d, J=1.9 Hz, 2H), 4.88-4.79 (m, 2H), 4.73 (d, J=12.9 Hz, 1H), 4.59 (d, J=5.5 Hz, 2H), 4.45 (s, 1H), 4.27 (d, J=3.9 Hz, 1H), 4.03 (s, 1H), 3.76 (s, 6H), 3.48 (dd, J=10.6, 3.3 Hz, 1H), 3.38 (dd, J=10.6, 4.3 Hz, 1H), 3.07 (s, 2H), 2.44 (s, 1H), 1.12 (s, 9H); ¹³C NMR (101 MHz, Chloroform-d) δ 177.6, 164.7, 158.5, 152.7, 151.6, 149.6, 144.5, 143.6, 141.9, 138.3, 135.7, 135.6, 133.7, 132.8, 130.1, 130.1, 128.8, 128.2, 127.9, 126.9, 123.5, 122.9, 117.1, 113.2, 87.0, 86.6, 84.5, 81.9, 69.8, 67.6, 64.3, 63.2, 55.3, 55.2, 53.5, 49.8, 38.7, 29.0, 26.8.

### (5-(2-(4-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methyl)-1H-1,2,3-triazol-1-yl)ethyl)-1H-imidazol-1-yl)methyl pivalate

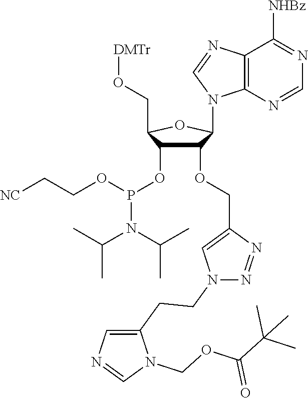

Following general procedure B, the title compound was afforded as a colorless foam (77%). Further purification was not required. ¹H NMR (400 MHz, CDCl₃) δ 9.23 (d, J=5.9 Hz, 1H), 8.69 (d, J=6.1 Hz, 1H), 8.13 (d, J=8.5 Hz, 1H), 8.08-7.92 (m, 2H), 7.63-7.57 (m, 1H), 7.55-7.47 (m, 3H), 7.44-7.36 (m, 2H), 7.33-7.14 (m, 7H), 6.86-6.77 (m, 4H), 6.75 (dd, J=4.2, 1.2 Hz, 1H), 6.17 (dd, J=6.7, 5.8 Hz, 1H), 5.68 (d, J=3.1 Hz, 2H), 4.91 (dt, J=11.2, 5.4 Hz, 1H), 4.82 (dd, J=12.6, 7.3 Hz, 1H), 4.69 (ddd, J=20.8, 11.8, 3.7 Hz, 2H), 4.54 (td, J=7.3, 2.1 Hz, 2H), 4.39 (dd, J=24.6, 3.7 Hz, 1H), 3.97-3.81 (m, 1H), 3.77 (dd, J=3.1, 0.7 Hz, 6H), 3.73-3.47 (m, 2H), 3.33 (ddd, J=10.6, 4.2, 1.8 Hz, 1H), 3.10-3.02 (m, 2H), 2.94 (s, 1H), 2.87 (d, J=0.6 Hz, 1H), 2.66-2.60 (m, 1H), 2.37 (dd, J=12.6, 6.2 Hz, 1H), 1.26 (dd, J=6.8, 5.6 Hz, 1H), 1.17 (d, J=6.8 Hz, 8H), 1.12 (d, J=1.5 Hz, 9H), 1.04 (d, J=6.8 Hz, 4H); ¹³C NMR (101 MHz, CDCl₃) δ 177.6, 164.7, 158.5, 152.7, 151.7, 149.5, 144.4, 144.1, 143.9, 142.1, 138.4, 138.2, 135.7, 135.6, 135.5, 133.8, 132.7, 130.1, 130.1, 128.8, 128.3, 128.2, 127.9, 126.9, 123.6, 122.8, 122.6, 117.9, 117.4, 116.9, 113.2, 87.0, 86.9, 86.7, 86.6, 84.4, 84.2, 80.1, 79.6, 71.2, 70.7, 70.6, 67.6, 64.2, 63.1, 62.8, 59.0, 58.9, 58.1, 57.9, 55.2, 49.6, 43.4, 43.3, 43.1, 38.7, 36.5, 29.1, 29.1, 26.8, 24.8, 24.7, 24.6, 20.4, 20.4, 20.2; ³¹P NMR (162 MHz, CDCl₃) δ 150.7, 150.4.

### Ethyl (E)-N,N′-dibenzoylcarbamimidothioate

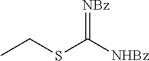

Following a procedure outlined in the art (H. Schotte, Verfahren zur Darstellung von Diacylisothioharnstoff-S-alkylaethern, DE1925C036959D 19250717, 1928.), (ethylsulfanyl)methanimidamide hydrobromide (21.3 g, 115 mmol) was dissolved in dry pyridine (110 mL) and benzoyl chloride (32.3 g, 230 mmol) was added at 0° C. Upon completion, the reaction mixture was poured into water. The formed crystals were filtered off and washed with EtOH and Et₂O, affording the title compound (29.7 g, 82%). ¹H NMR (400 MHz, DMSO) δ 13.58 (s, 1H), 8.25-8.11 (m, 2H), 7.94-7.84 (m, 2H), 7.75-7.48 (m, 6H), 3.37 (s, 1H), 3.19 (q, J=7.3 Hz, 2H), 1.37 (t, J=7.3 Hz, 3H); ¹³C NMR (101 MHz, DMSO) δ 175.1, 167.9, 164.5, 135.6, 133.4, 132.8, 131.5, 129.5, 128.9, 128.3, 127.6, 25.1, 13.4. LRMS ES+ (m/z): [M]⁺ calc'd for C₁₇H₁₆N₂O₂S; 312.4 found: 313.1 [M+H]⁺

### (Z)-N-(N-(4-azidobutyl)-N′-benzoylcarbamimidoyl)benzamide

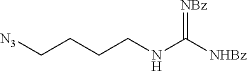

To a stirred solution of ethyl (E)-N,N-dibenzoylcarbamimidothioate (8.43 g, 27 mmol) in MeCN (50 mL), 4-azidobutan-1-amine (3.49 g, 30 mmol) was added. The reaction mixture was stirred at ambient temperature for 2 h and concentrated in vacuo. The crude mixture was purified by column chromatography DCM/Pentane (20-100%). Affording the title compound (9.01 g, 82%) ¹H NMR (400 MHz, DMSO) δ 14.38 (s, 1H), 9.54 (t, J=5.9 Hz, 1H), 8.26-8.18 (m, 2H), 8.02-7.94 (m, 2H), 7.80-7.72 (m, 1H), 7.72-7.63 (m, 2H), 7.62-7.53 (m, 1H), 7.49 (ddt, J=8.2, 6.7, 1.2 Hz, 2H), 3.67 (q, J=6.6 Hz, 2H), 3.42 (t, J=6.7 Hz, 2H), 1.82-1.71 (m, 2H), 1.66 (dddd, J=13.7, 8.2, 6.6, 1.7 Hz, 2H); ¹³C NMR (101 MHz, DMSO) δ 177.6, 167.5, 156.8, 137.9, 134.2, 132.5, 132.4, 129.9, 129.6, 128.6, 127.9, 50.8, 40.8, 26.4, 26.2. LRMS ES+ (m/z): [M]⁺ calc'd for C₁₉H₂₀N₆O₂; 364.4 found: 365.1 [M+H]⁺.

### N-((Z)-benzamido((4-(4-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methyl)-1H-1,2,3-triazol-1-yl)butyl)amino)methylene)benzamide

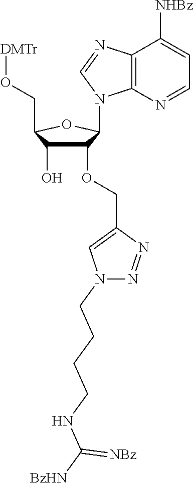

Following general procedure A, the title compound was afforded as a colorless foam (40%) after purification by column chromatography (1 to 10% MeOH/DCM). ¹H NMR (400 MHz, DMSO) δ 14.35 (s, 1H), 11.24 (s, 1H), 9.51 (t, J=5.9 Hz, 1H), 8.66 (s, 1H), 8.54 (s, 1H), 8.21-8.14 (m, 2H), 8.07-8.02 (m, 2H), 8.01 (s, 1H), 7.98-7.94 (m, 2H), 7.76-7.70 (m, 1H), 7.69-7.61 (m, 3H), 7.57-7.50 (m, 3H), 7.48 (m, 2H), 7.38-7.33 (m, 2H), 7.28-7.16 (m, 7H), 6.87-6.78 (m, 4H), 6.19 (d, J=4.8 Hz, 1H), 5.76 (s, 1H), 5.42 (d, J=6.1 Hz, 1H), 4.83 (t, J=5.0 Hz, 1H), 4.76 (d, J=12.3 Hz, 1H), 4.67 (d, J=12.3 Hz, 1H), 4.54 (q, J=5.3 Hz, 1H), 4.36 (t, J=7.0 Hz, 2H), 4.14 (q, J=4.6 Hz, 1H), 3.71 (d, J=1.1 Hz, 6H), 3.64 (q, J=6.6 Hz, 2H), 3.36 (s, 8H), 3.25 (d, J=4.8 Hz, 2H), 1.88 (p, J=7.1 Hz, 2H), 1.69-1.58 (m, 2H); ¹³C NMR (101 MHz, DMSO) δ 176.8, 166.6, 165.2, 157.6, 155.9, 151.5, 151.3, 150.1, 144.4, 143.2, 142.7, 137.0, 135.2, 135.0, 133.3, 132.9, 132.0, 131.6, 131.5, 129.3, 129.0, 128.7, 128.1, 128.0, 127.8, 127.4, 127.3, 127.1, 126.2, 125.4, 123.5, 112.7, 85.9, 83.4, 79.2, 68.7, 63.1, 62.8, 54.6, 54.5, 48.6, 26.7, 25.3. LRMS ES+ (m/z): [M]⁺ calc'd for C₆₀H₅₇N₁₁O₉; 1076.2 found: 774.2 [M+2H-DMTr]⁺.

### (2R,3R,4R,5R)-5-(6-benzamido-9H-purin-9-yl)-2-((bis(4-methoxyphenyl)(phenyl)methoxy)methyl)-4-((1-(4-((Z)-2,3-dibenzoylguanidino)butyl)-1H-1,2,3-triazol-4-yl)methoxy)tetrahydrofuran-3-yl (2-cyanoethyl) diisopropylphosphoramidite

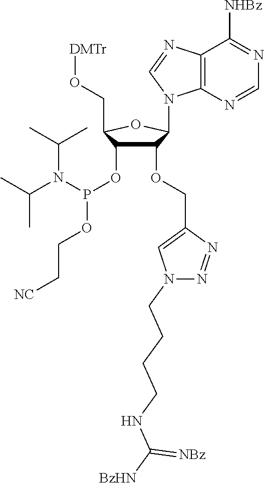

Following general procedure B, the title compound was afforded as a colorless foam (90%) following purification by column chromatography (60% EtOAc/pentane). ¹H NMR (400 MHz, CDCl₃) δ 14.50-14.46 (m, 1H), 9.51 (s, 1H), 9.03 (s, 1H), 8.70 (d, J=5.6 Hz, 1H), 8.27-8.20 (m, 2H), 8.15 (d, J=8.3 Hz, 1H), 8.06-7.96 (m, 4H), 7.67-7.58 (m, 1H), 7.62-7.37 (m, 11H), 7.37-7.22 (m, 6H), 7.26-7.15 (m, 1H), 6.85-6.75 (m, 4H), 6.19 (t, J=5.4 Hz, 1H), 4.95 (dt, J=13.3, 5.3 Hz, 1H), 4.86 (dd, J=12.5, 6.8 Hz, 1H), 4.79-4.62 (m, 2H), 4.47-4.35 (m, 1H), 4.38-4.28 (m, 2H), 3.97-3.79 (m, 1H), 3.77 (dd, J=2.5, 0.6 Hz, 6H), 3.73-3.63 (m, 2H), 3.66-3.48 (m, 3H), 3.35 (ddd, J=10.6, 4.3, 1.8 Hz, 1H), 2.62 (t, J=6.3 Hz, 1H), 2.38 (t, J=6.4 Hz, 1H), 2.04 (s, 3H), 2.04-1.94 (m, 2H), 1.92 (s, 1H), 1.71 (q, J=7.4, 6.9 Hz, 2H), 1.20-1.12 (m, 9H), 1.05 (d, J=6.8 Hz, 3H); ¹³C NMR (101 MHz, CDCl₃) δ 178.6, 168.4, 164.6, 158.6, 157.2, 152.6, 151.8, 149.5, 144.4, 142.1, 142.0, 137.6, 135.7, 133.6, 132.8, 132.1, 131.9, 130.1, 129.4, 129.2, 128.9, 128.3, 128.2, 128.1, 127.9, 127.9, 127.8, 127.0, 122.7, 122.4, 118.0, 113.2, 87.1, 86.6, 84.3, 80.1, 63.0, 62.8, 60.4, 59.0, 58.8, 55.3, 55.2, 49.8, 43.4, 43.3, 43.1, 40.4, 27.6, 26.3, 24.8, 24.7, 24.6, 21.1, 20.4, 20.2, 14.2; ³¹P NMR (162 MHz, CDCl₃) δ 150.64, 150.34.

### (9H-fluoren-9-yl)methyl (S)-(1-azido-19-oxo-21-(1-trityl-1H-imidazol-4-yl)-3,6,9,12,15-pentaoxa-18-azahenicosan-20-yl)carbamate

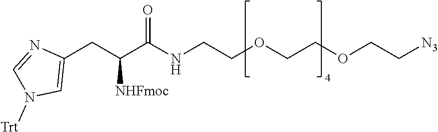

Na-(((9H-fluoren-9-yl)methoxy)carbonyl)-Nt-trityl-L-histidine (185 mg, 299 μmol) was activated with HATU (125 mg, 329 μmol), 1H-benzo[d][1,2,3]triazol-1-ol hydrate (50.4 mg, 329 μmol) and DIPEA (116 mg, 898 μmol) in DMF (2 mL) for 20 min. A solution of 17-azido-3,6,9,12,15-pentaoxaheptadecan-1-amine (110 mg, 359 μmol) was then added in DMF (1 mL). The reaction mixture was stirred for 3 h. When complete (TLC: 10% MeOH/DCM), the reaction mixture was extracted with Et₂O (3×10 mL) and DCM (10 mL). The combined organic mixtures were then concentrated and the crude residue was purified by flash chromatography with a gradient of 50% EtOAc/pentane to 10% MeOH/DCM, affording the title compound as a colorless oil (203 mg, 75%). LRMS ES+ (m/z): [M]⁺ calc'd for C₅₂H₅₇N₇O₈: 908.1; found: 930.5 ([M+Na]⁺).

### (S)-2-amino-N-(17-azido-3,6,9,12,15-pentaoxaheptadecyl)-3-(1-trityl-1H-imidazol-4-yl)propanamide

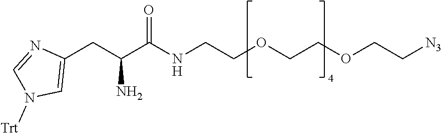

(9H-fluoren-9-yl)methyl(S)-(1-azido-19-oxo-21-(1-trityl-1H-imidazol-4-yl)-3,6,9,12,15-pentaoxa-18-azahenicosan-20-yl)carbamate (203 mg, 262 μmol) was dissolved in a 1:1 mixture of DCM/diethylamine (2 mL) and was stirred at RT for 90 min. Upon completion (TLC: 10% MeOH/DCM), the reaction mixture was concentrated in vacuo and was resuspended in DCM, followed by further concentration (×2). The crude residue was then purified by column chromatography (5 to 10% MeOH/DCM) to afford the title compound as a colorless foam (101 mg, 66%). LRMS ES+ (m/z): [M]⁺ calc'd for C₃₇H₄₇N₇O₆: 685.8; found: 686.4.

### (S)-2-amino-N-(17-azido-3,6,9,12,15-pentaoxaheptadecyl)-3-(1H-imidazol-4-yl)propanamide

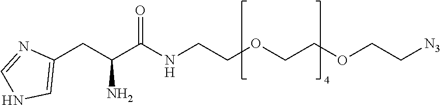

(S)-2-amino-N-(17-azido-3,6,9,12,15-pentaoxaheptadecyl)-3-(1-trityl-1H-imidazol-4-yl)propanamide (101 mg, 147 μmol) was dissolved in a 3:1 mixture of 4 M HCl in dioxane/MeOH and the reaction mixture was stirred at 60° C. for 2 h. Upon full conversion (TLC: 10% MeOH/DCM), the solvent was removed under a stream of nitrogen and the crude residue was triturated in Et₂O, affording the title compound as a colorless foam (69 mg, 98%). ¹H NMR (400 MHz, CDCl₃) δ 13.89 (s, 1H), 13.49 (s, 1H), 8.96 (s, 1H), 8.61 (s, 1H), 8.33 (s, 2H), 7.65 (s, 1H), 4.70 (s, 1H), 3.81-3.63 (m, 18H), 3.43 (s, 2H), 3.35-3.08 (m, 4H); LRMS ES+ (m/z): [M]⁺ calc'd for C₁₈H₃₃N₇O₆: 443.7; found: 444.7.

### 1-(17-azido-3,6,9,12,15-pentaoxaheptadecyl)guanidine

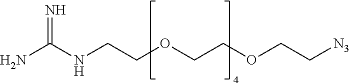

To a stirred solution of pyrazole-1-carboxamidine hydrochloride (374 mg, 2.55 mmol) and DIPEA (356 mg, 479 μL, 2.75 mmol) in DCM (2 mL), 17-azido-3,6,9,12,15-pentaoxaheptadecan-1-amine (766 mg, 2.50 mmol) was added. The reaction mixture was stirred at RT for 26 h and, upon completion, was concentrated in vacuo. The crude residue was then purified by column chromatography (5 to 10% 7N NH3 in MeOH/DCM to 30% to overcome issues with dragging on the column). The title compound was isolated as a brown oil. ¹H NMR (400 MHz, DMSO) δ 7.85-6.92 (m, 2H), 3.64-3.46 (m, 20H), 3.40 (dd, J=5.6, 4.3 Hz, 2H), 3.29 (q, J=5.3 Hz, 2H); ¹³C NMR (101 MHz, DMSO) δ 157.8, 70.3, 70.2, 70.2, 70.2, 69.7, 69.1, 50.5, 41.3. LRMS ES+ (m/z): [M]⁺ calc'd for C₁₃H₂₈N₆O₅: 349.4; found: 350.4.

### Example 9—CuAAC to Install Modifications on Oligonucleotides

### General Procedure for Internal Click Modifications

Click reactions were performed in 1:1 mixtures 2 M TEAA:DMSO using standard protocols with Cu₂SO₄ and TTIPTA (E. Paredes, S. R. Das, Click chemistry for rapid labeling and ligation of RNA, ChemflioChem. 12 (2011) 125-131).

TABLE 11
Theoretical Mw values and experimental Mw for some representative
clicked products

| | Number of | Mw Starting | Theoretical | |
|---|---|---|---|---|
| Sample | modifications | Material | Mw | Mw found |
| 1 | 8 | 19644 | 20437.12 | 20436 |
| 2 | 19 | 20062 | 21945.66 | 21945 |
| 3 | 12 | 15696 | 17579.1 | 17578 |
| 4 | 12 | 15696 | 16905.9 | 16905 |
| 5 | 8 | 19644 | 22431.2 | 22431 |
| 6 | 19 | 20062 | 26681.6 | 26682 |
| 9 | 12 | 15969 | 19896.3 | 19896 |
| 10 | 8 | 19644 | 22530.3 | 22531 |
| 11 | 164-1 | 15940 | 17130.4 | 17130 |
| 12 | 165-1 | 16048 | 18065.6 | 18066 |

### Example 10—Peptide Synthesis and RNA Conjugation

### Solid Phase Peptide Synthesis—Example Procedure

The following solutions were prepared: Deprotection solution: 20% piperidine in DMF; Activator solution: 0.25 M HATU in DMF; Basic solution: 2,6-lutidine (2.05 mL)+DIPEA (1.96 mL) in DMF (5.54 mL) Capping solution: Ac₂O (0.92 mL)+2,6-lutidine (1.3 mL) in DMF (18 mL); Amino acid solution: 0.2 M in DMF.

Pre-loaded amino-based resin (as described above) (50 mg) was swelled in DMF (3 mL) at rt for 30 min. The DMF was then drained, and the resin was immersed in 20% piperidine in DMF (this step was repeated). The resin was then washed with DMF (3×3 mL), DCM (3×3 mL) and again with DMF (3×3 mL). In a separate vessel, the desired amino acid solution (1.29 mL), HATU (452 μL, 4.5 equiv.) and base solution (110 μL) were mixed and then added to the resin. The resultant suspension was then agitated at rt for 30 min, the syringe was flushed, and the coupling step was repeated. Coupling success was monitored with the Kaiser test. Following successful coupling, the resin was washed with DMF (3×3 mL), DCM (3×3 mL) and DMF (3×3 mL). The resin was then immersed in capping solution (vide supra) for 5 min. The syringe was flushed, and the resin was washed with DMF (3×3 mL), DCM (3×3 mL) and DMF (3×3 mL). The process was then repeated (from the deprotection step) until the desired sequence was obtained.

Cleavage from the resin was achieved by submerging it in a mixture of TFA/phenol/water/TIPS (88/5/5/2) and agitating for 3 h, followed by dropwise precipitation into ice cold diethyl ether. The resultant precipitate was then dissolved in acetic acid and lyophilized, affording the desired peptide as the acetate salt.

| | | Expected | Mass |
|---|---|---|---|
| Name | Sequence (N-C) | mass | found |
| HA2 | GLFGAIAGFIENGW | 2653.95 | 2655 |
| | EGMIDGWYG | | |
| GALA3 | LAEALAEALEALAA | 1548.76 | 1549 |
| H4 | HHHH | 759.56 | 758.8 |
| H8 | HHHHHHHH | 1308.12 | 1308.6 |
| H12 | HHHHHHHHHHHH | 1856.3 | 1857.6 |

These peptides may be added to any given design shown in FIG. 1, FIG. 4, and FIG. 6.

### Purification

Purification of RNA-peptide conjugates was carried out by IEX preparative HPLC using a PL-SAX (Agilent) 22×150 mm 1000 Å column at 75° C. with a flow rate of 15 mL/min and UV detection at 260 nm. Elution was performed with a linear gradient selected based on impurity profile, determined by analytical testing using either a DNAPac PA200RS UPLC column or PL-SAX analytical column. Buffer A: 25 mM Tris HCl, pH 8.0, 20% acetonitrile, 10 mM sodium perchlorate; buffer B: 25 mM Tris HCl, pH 8.0, 20% acetonitrile, 600 mM sodium perchlorate.

### Example 11—Oligophosphates for PK/PD Modulation

Although dispersing the modifications throughout a nucleic acid nanoparticle structure has its advantages, some modifications might not be suitable for internal positioning. In such instances, 5′ modification of the oligonucleotide can help mitigate any potential issues. 5′ modification has been shown extensively in the literature. The following examples are incorporated herein by reference and include small molecules (E. Paredes, M. Evans, S. R. Das, RNA labeling, conjugation and ligation, Methods. 54 (2011) 251-259), peptides (K. Klabenkova, A. Fokina, D. Stetsenko, Chemistry of peptide-oligonucleotide conjugates: A review, Molecules. 26 (2021) 1-36), polymers (F. Xiao, Z. Wei, M. Wang, A. Hoff, Y. Bao, L. Tian, Oligonucleotide-Polymer Conjugates: From Molecular Basics to Practical Application, Springer International Publishing, 2020) and lipids (X. Li, K. Feng, L. Li, L. Yang, X. Pan, H. S. Yazd, C. Cui, J. Li, L. Moroz, Y. Sun, B. Wang, X. Li, T. Huang, W. Tan, Lipid-oligonucleotide conjugates for bioapplications, Natl. Sci. Rev. 7 (2020) 1933-1953).

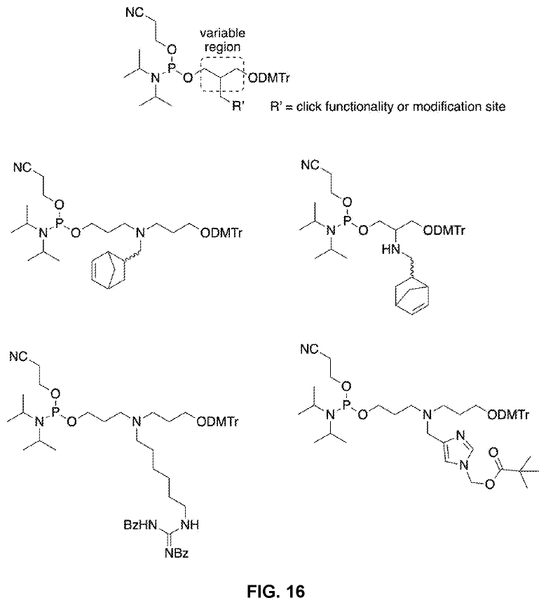

The structure-function relationship of oligonucleotides has inspired the development of alternative functional materials that utilize the phosphodiester backbone (N. Appukutti, C. J. Serpell, High definition polyphosphoesters: Between nucleic acids and plastics, Polym. Chem. 9 (2018) 2210-2226). Rather than forming chains of nucleotides, functional monomers are linked together with phosphodiesters via phosphoramidite chemistry. Although there are size limitations to these polymers, it is possible to get completely monodisperse, sequence-defined materials in good yields. A powerful way of introducing modifications at the oligonucleotide level is through the introduction of oligophosphate polymers at the 5′ end (FIG. 6). These polymers may consist of click functionalities and will provide a long chain for post-synthetic modification (FIG. 16). Upon conjugation of the click-functionalized chain, standard click procedures may be used to introduce any given modification.

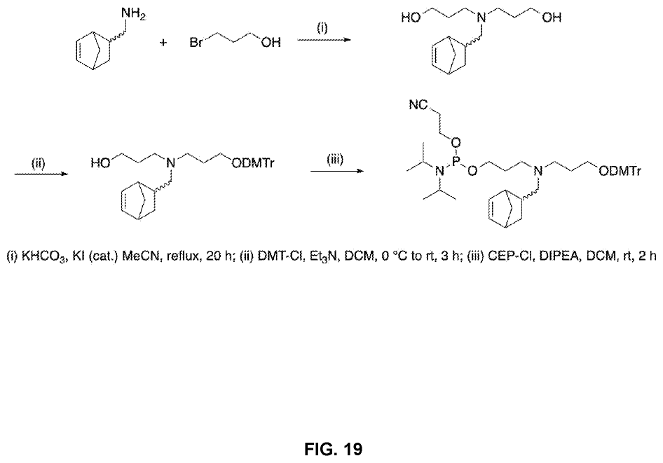

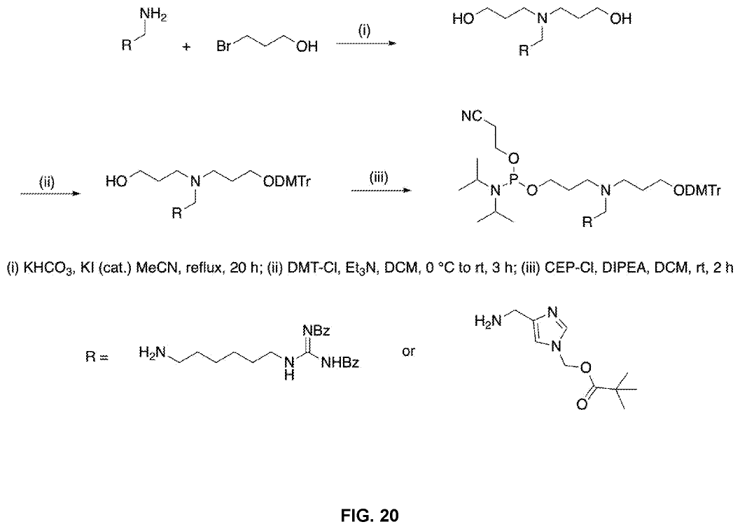

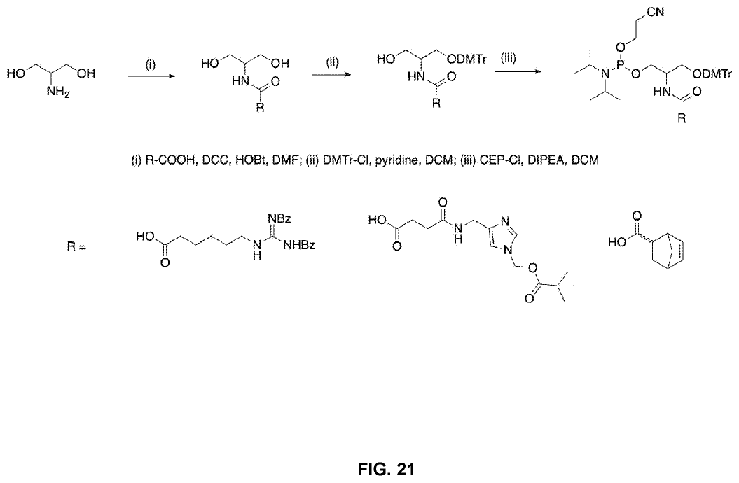

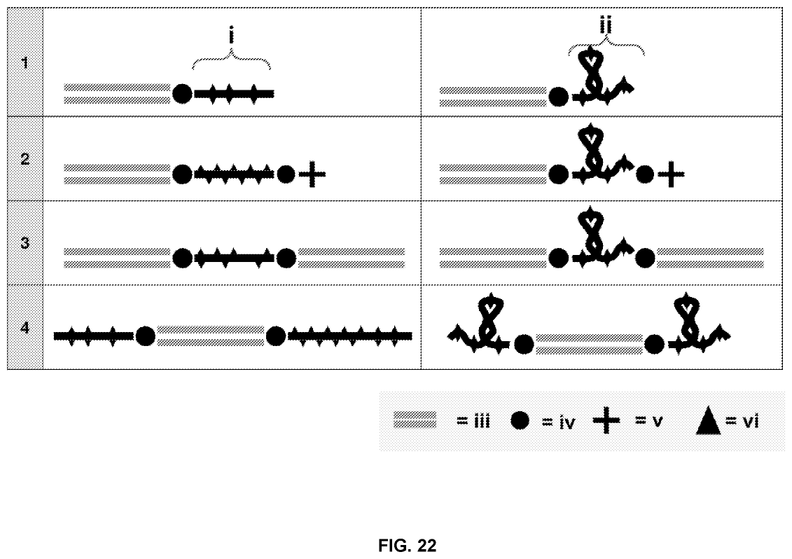

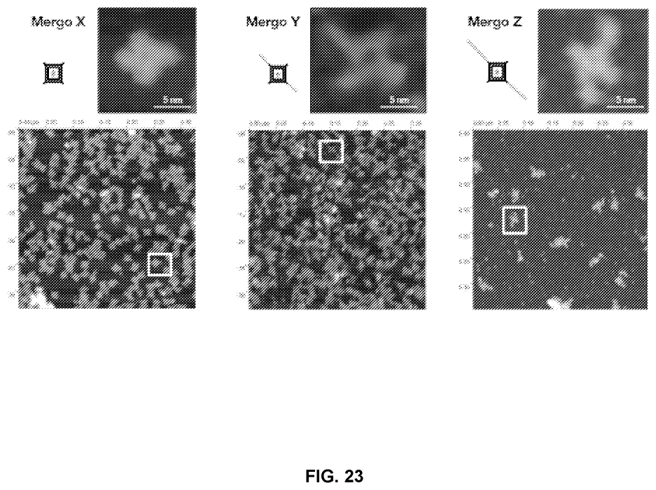

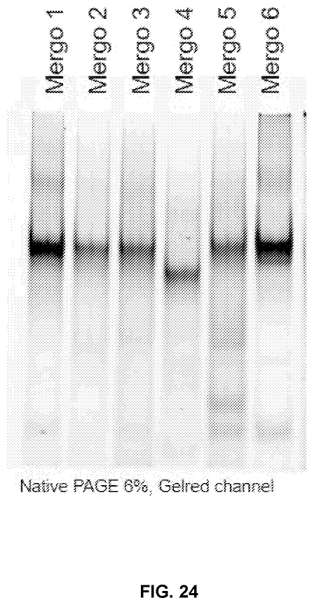

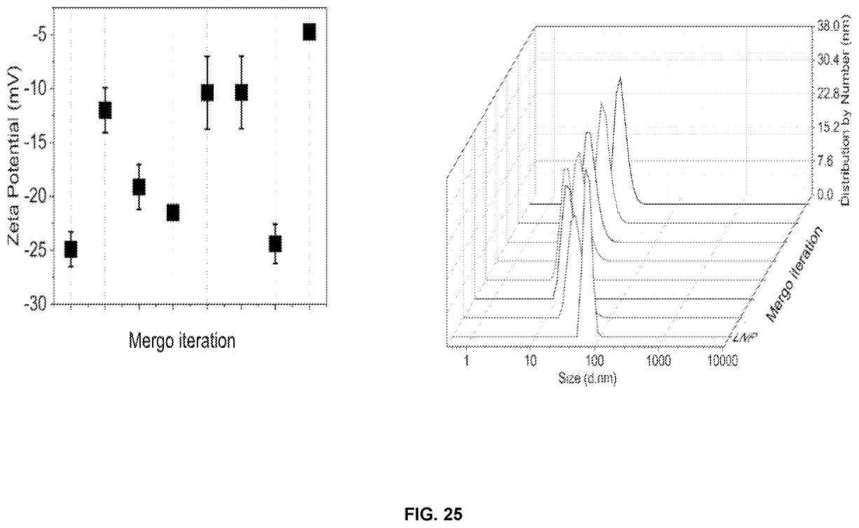

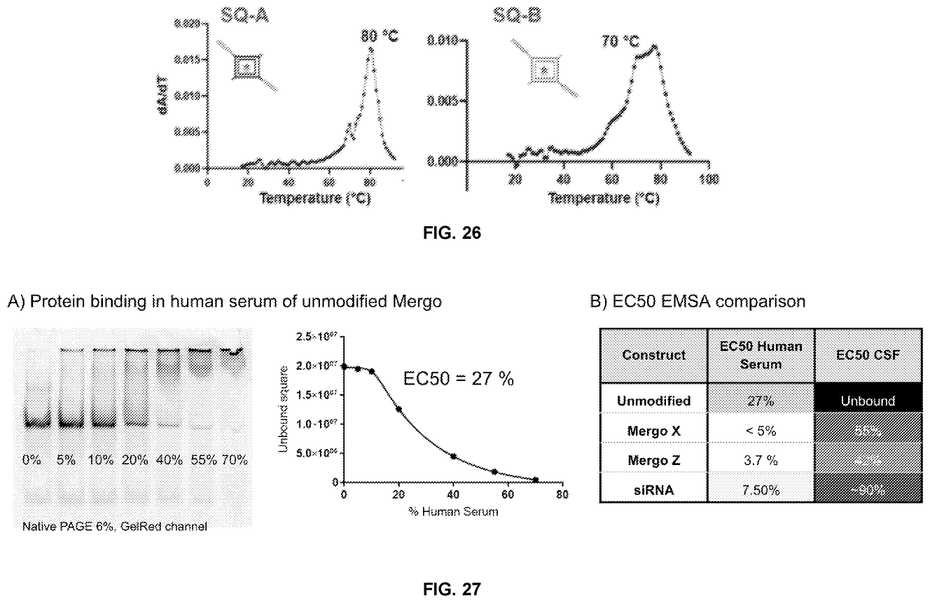

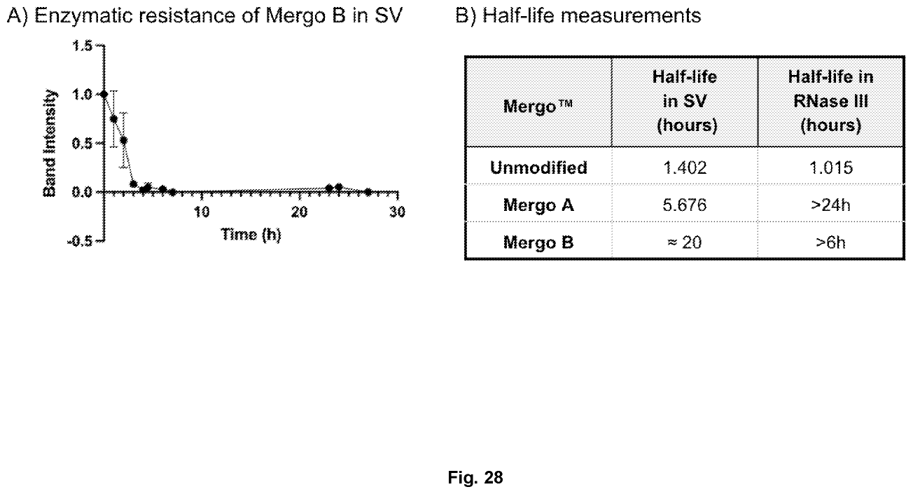

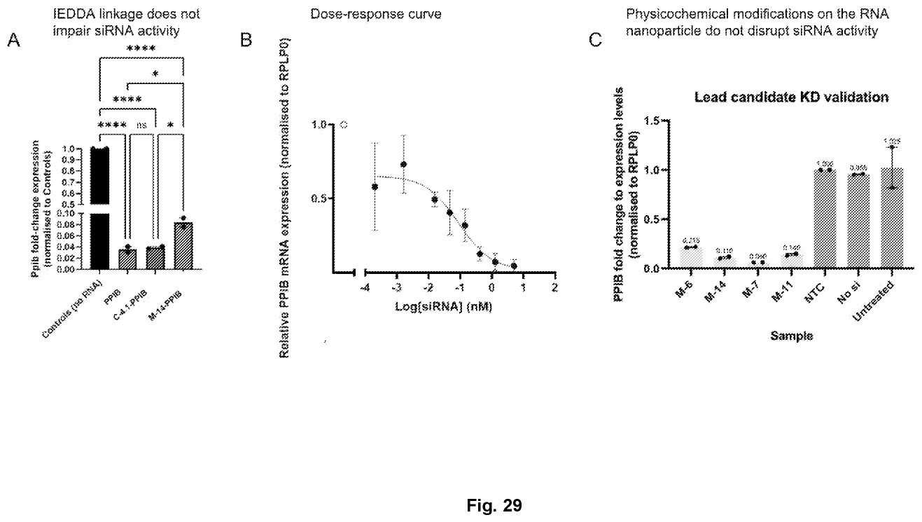

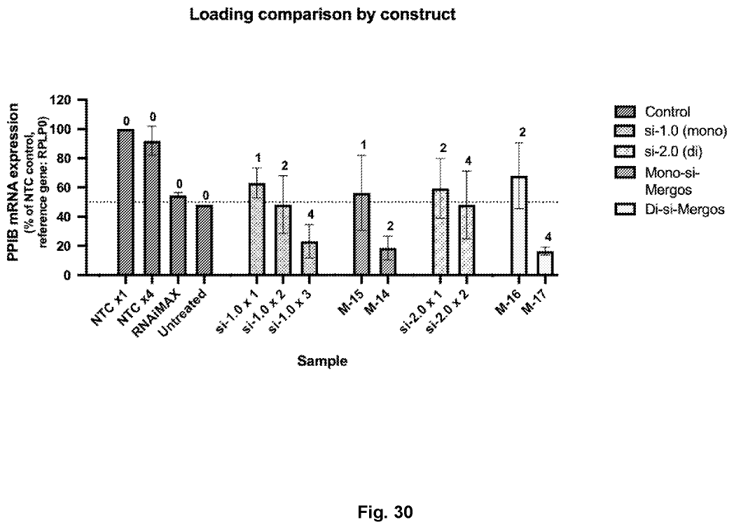

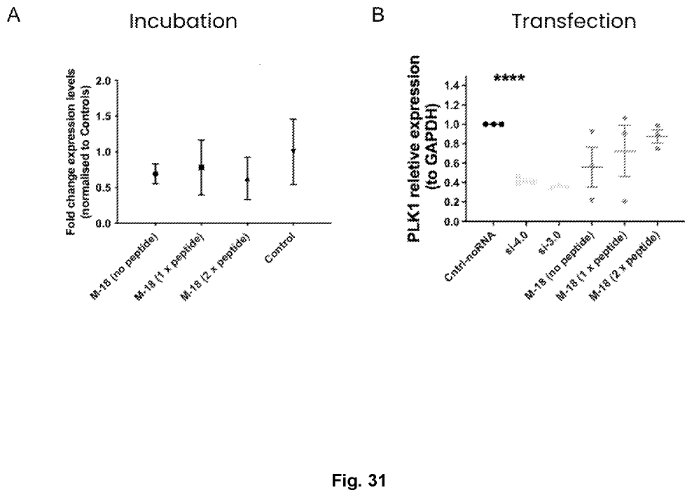

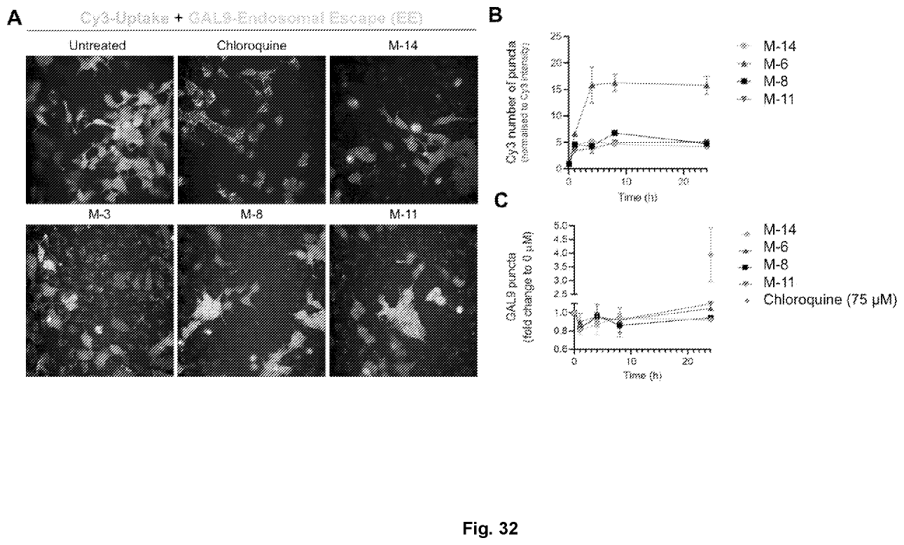

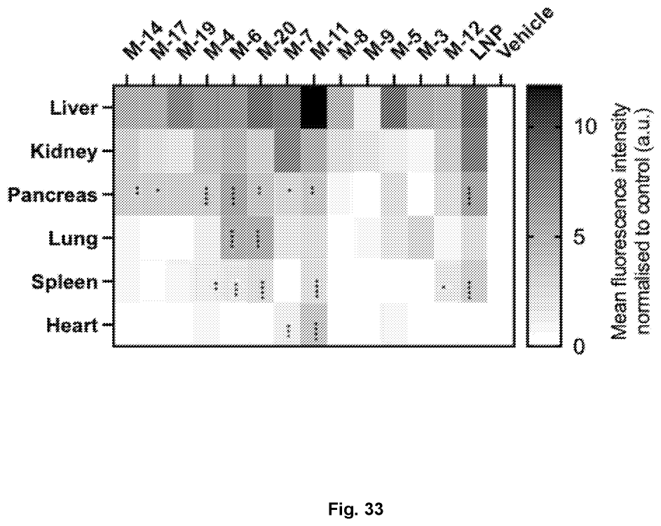

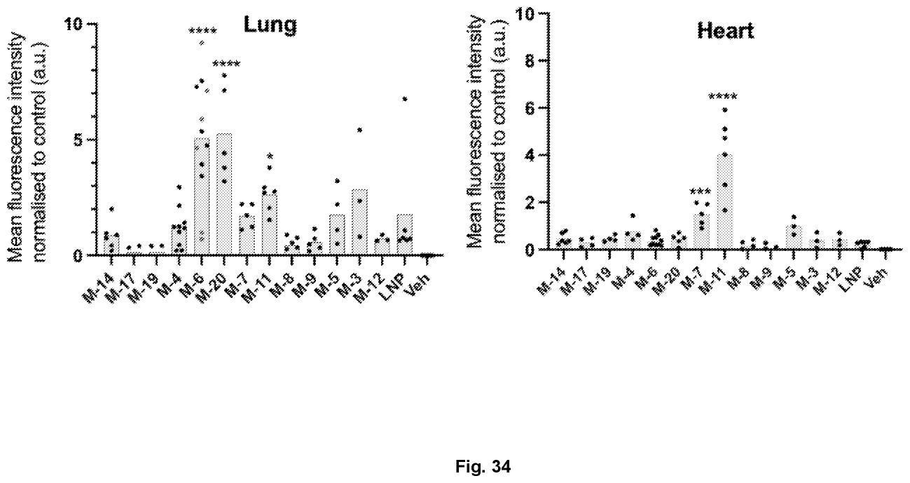

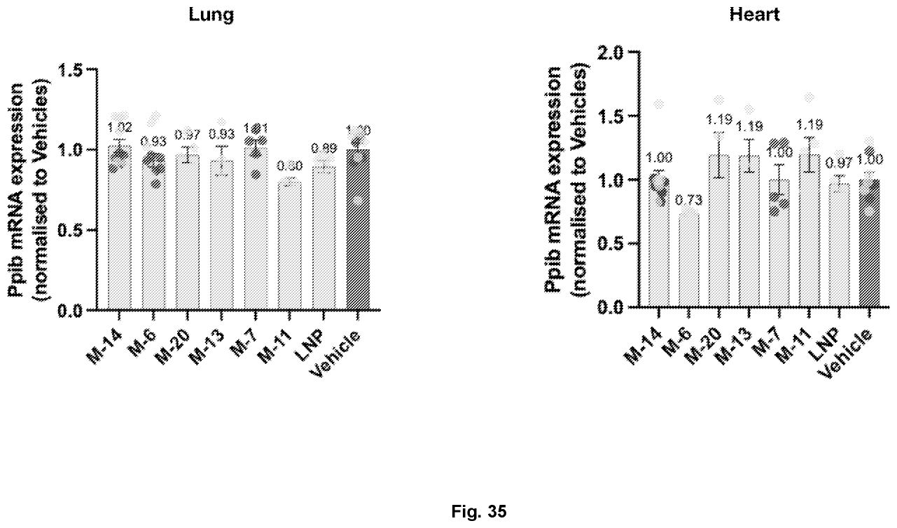

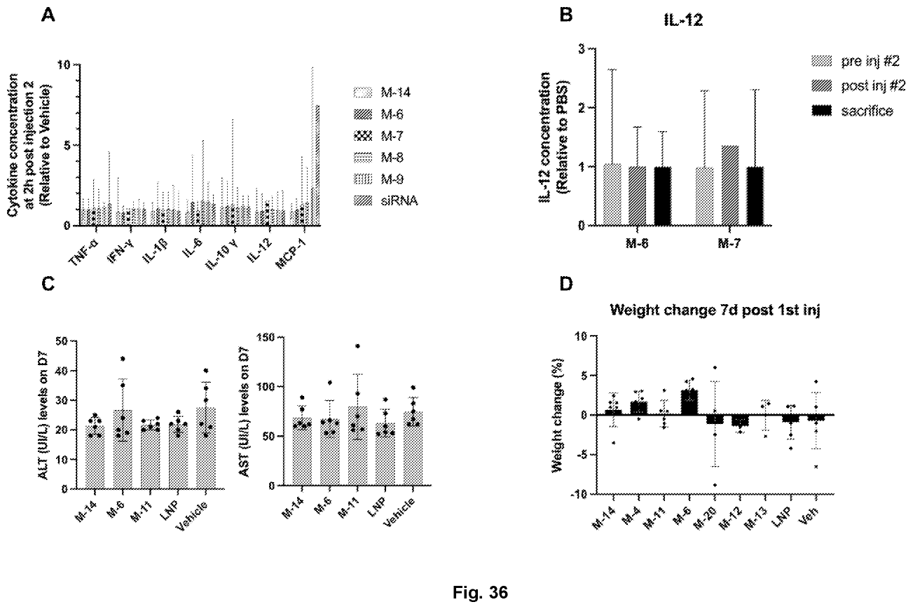

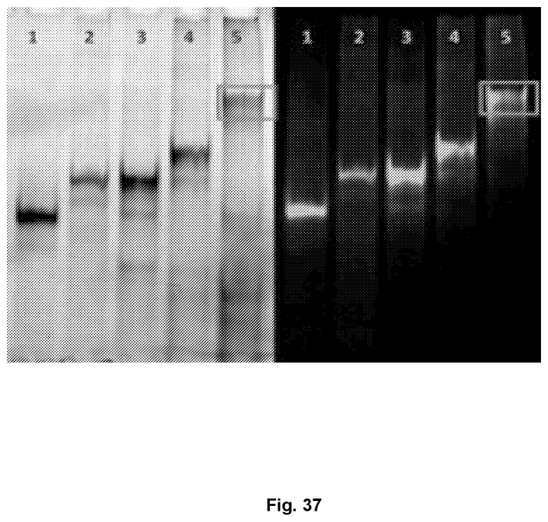

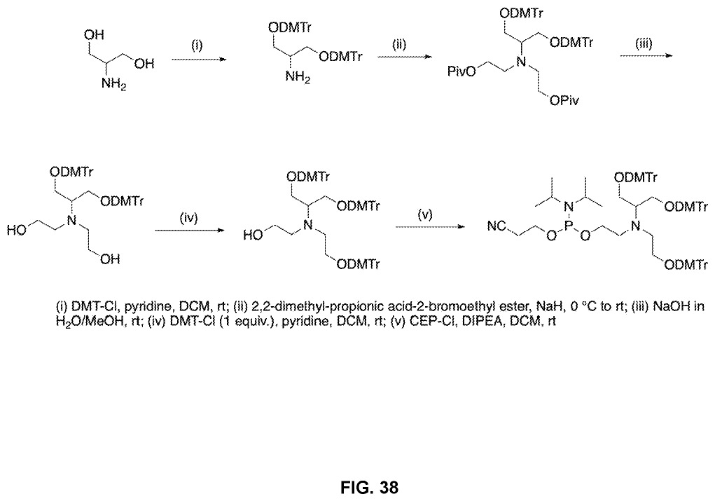

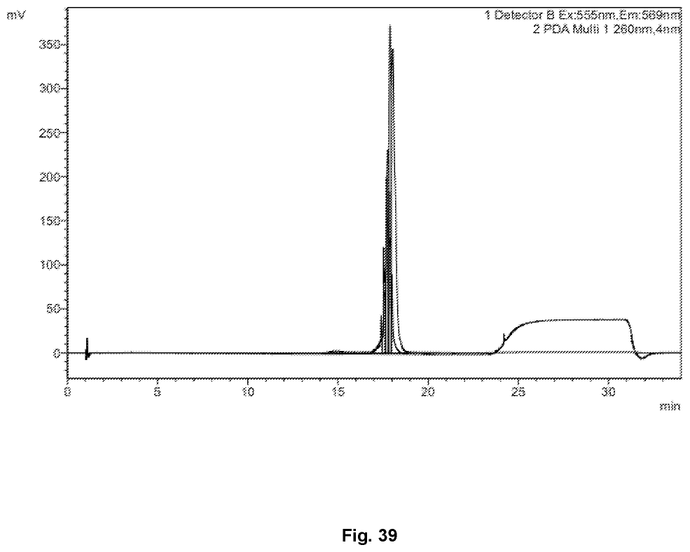

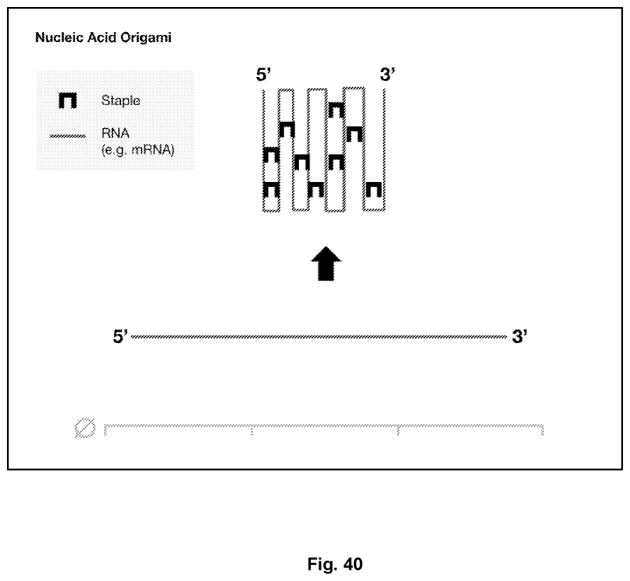

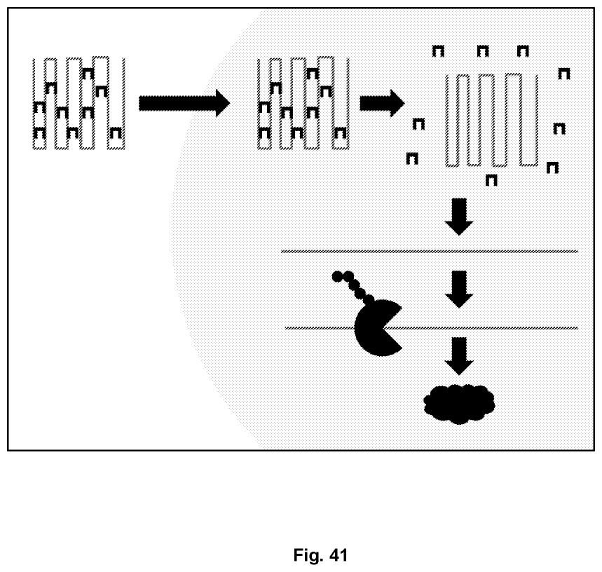

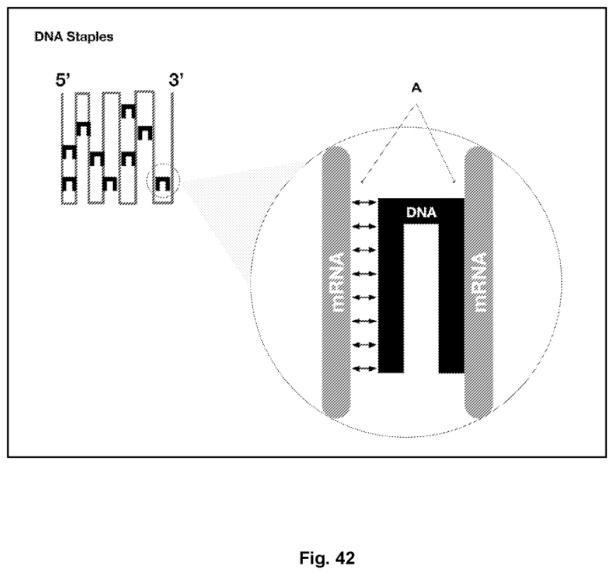

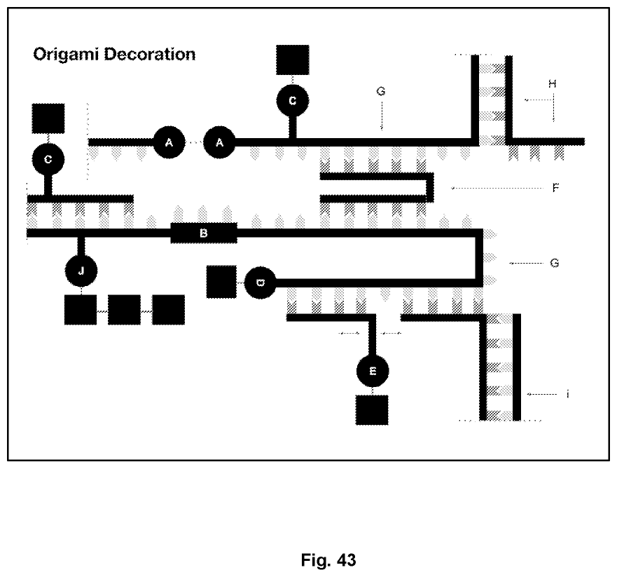

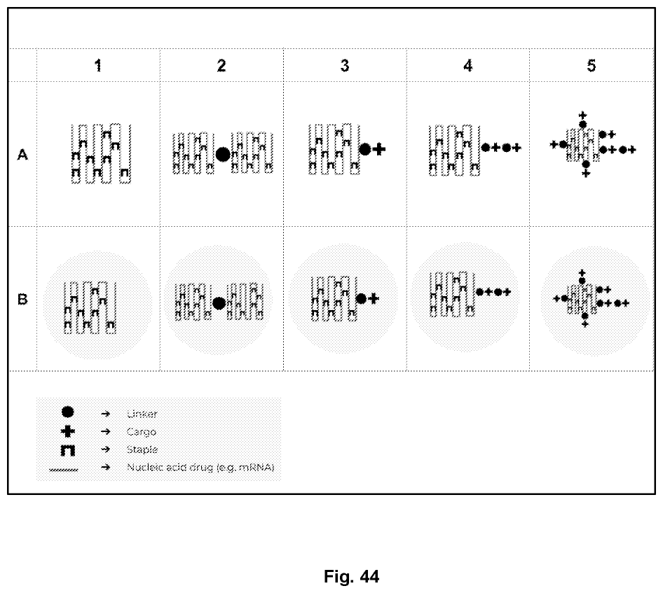

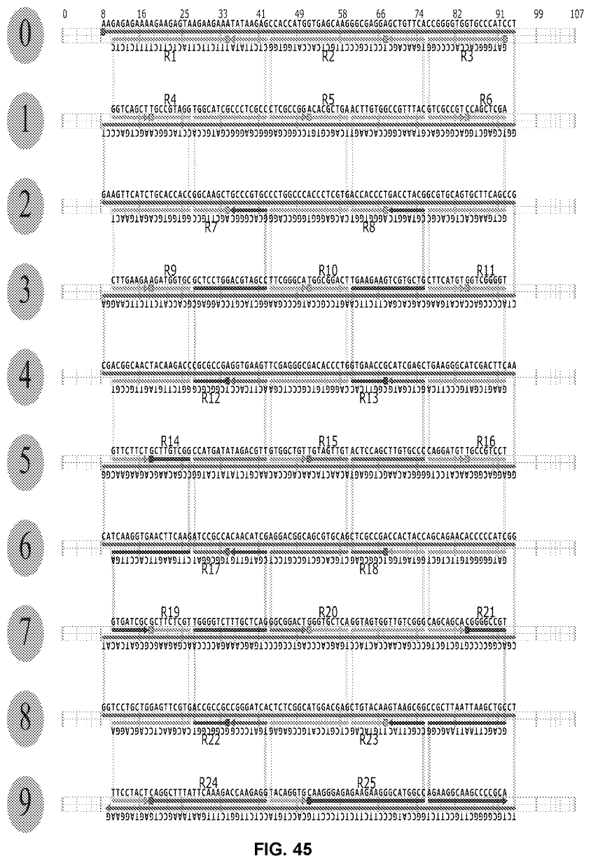

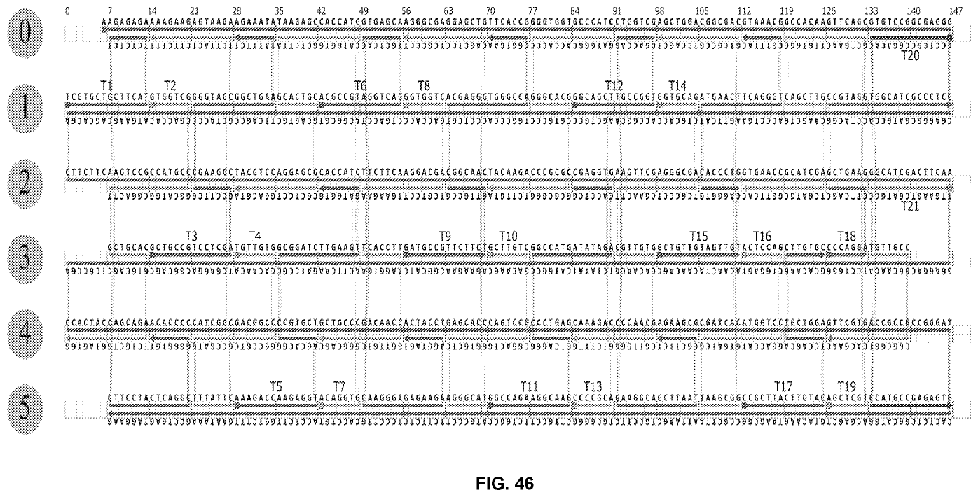

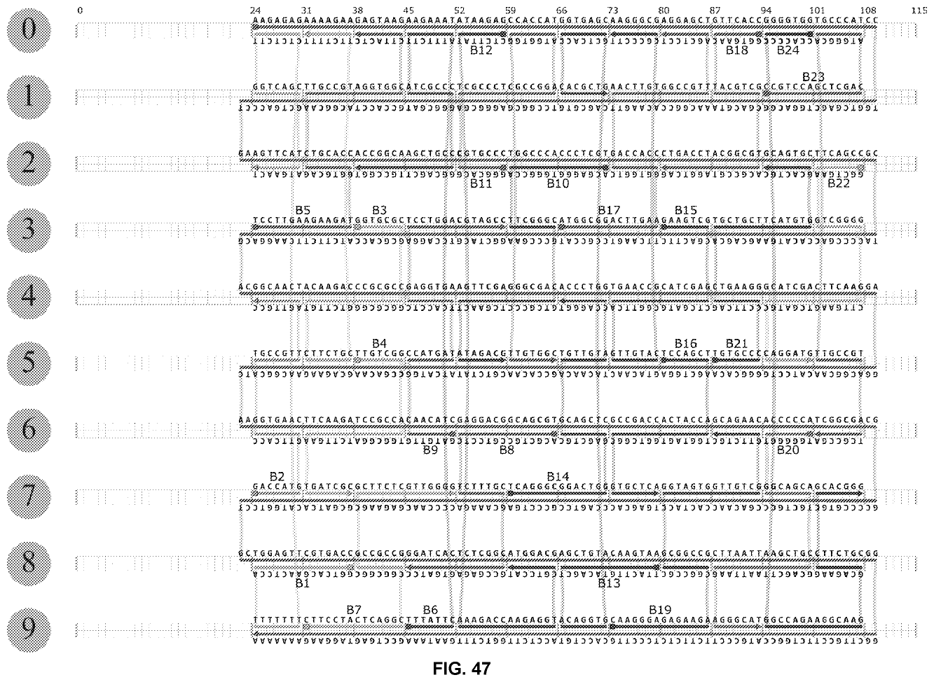

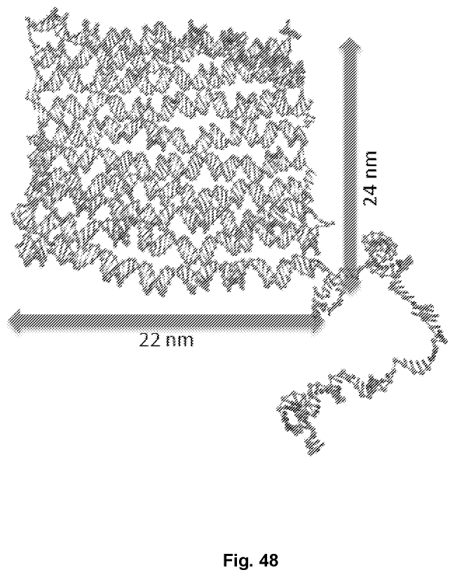

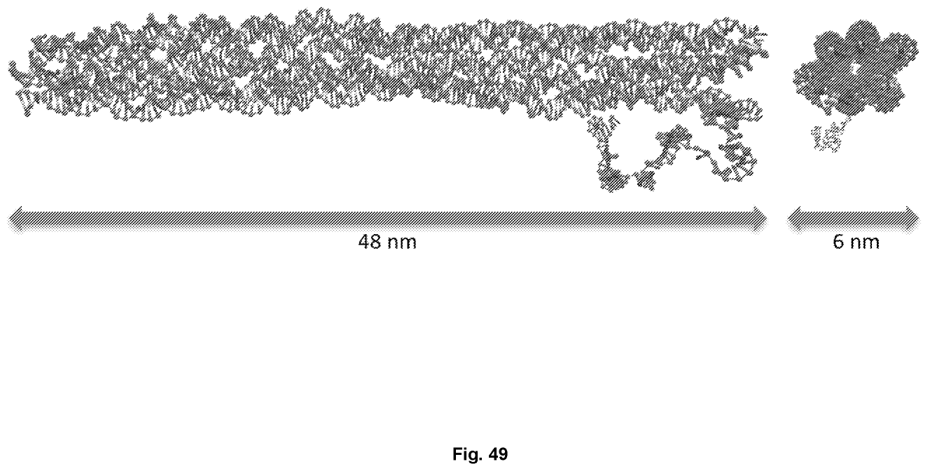

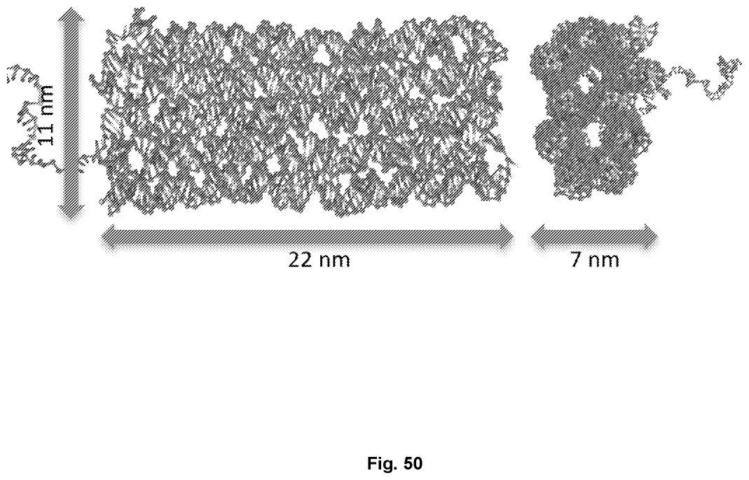

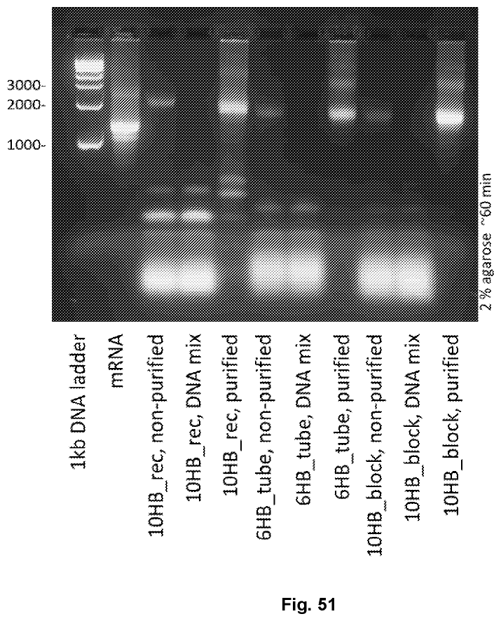

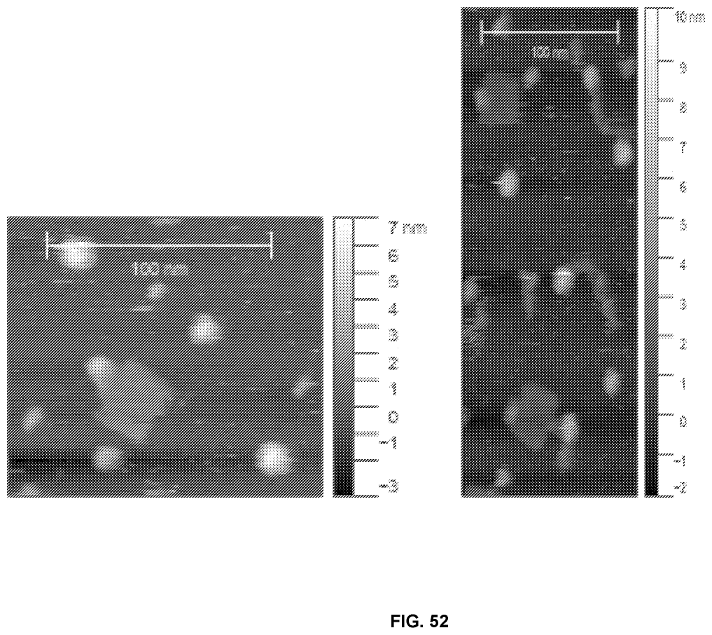

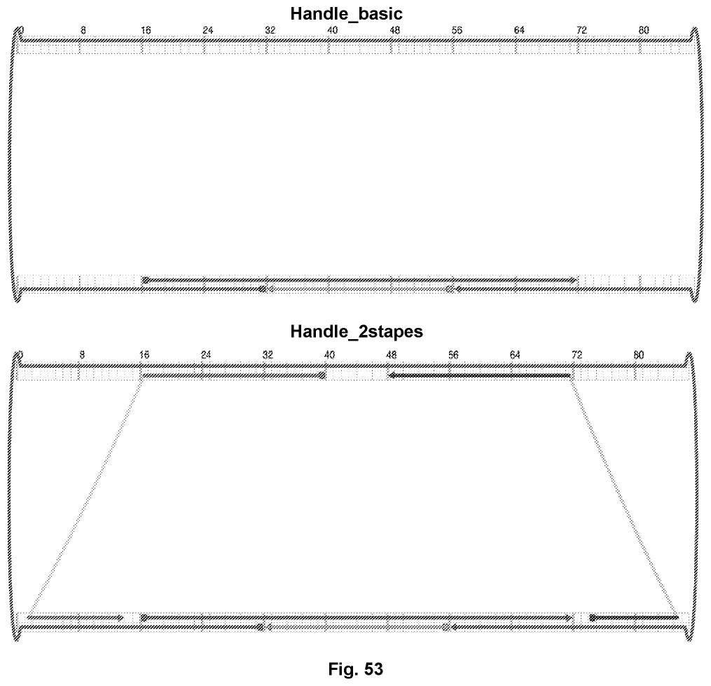

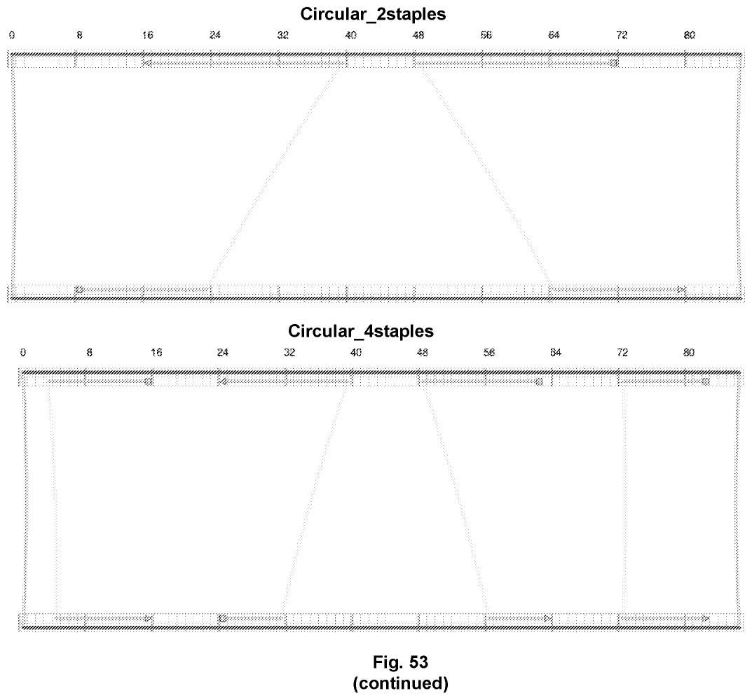

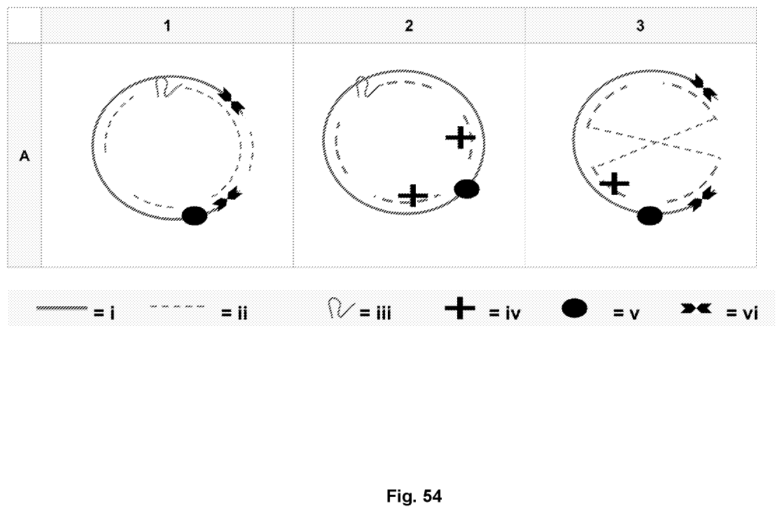

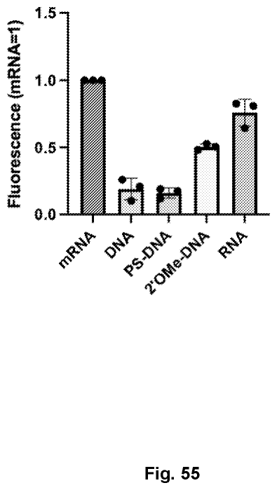

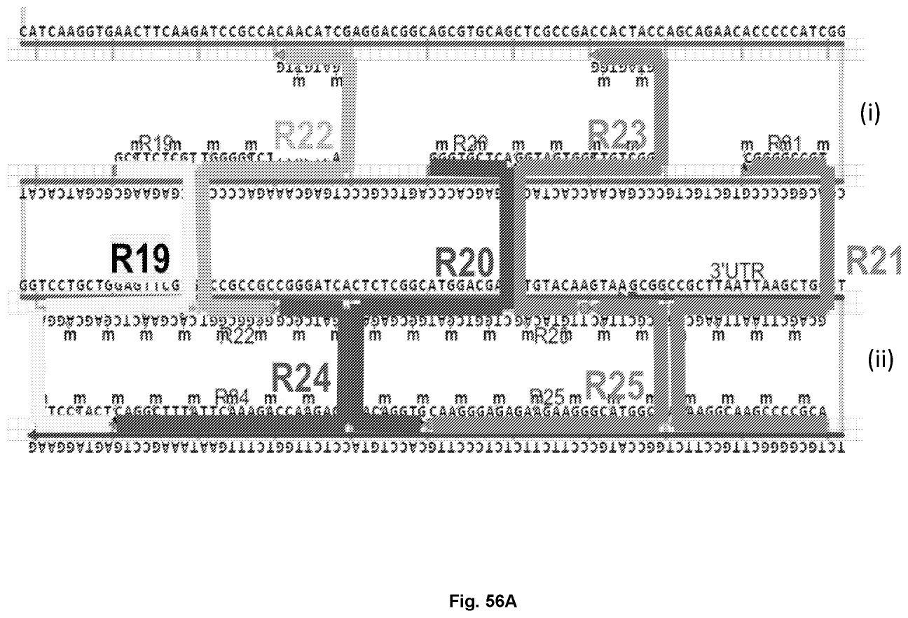

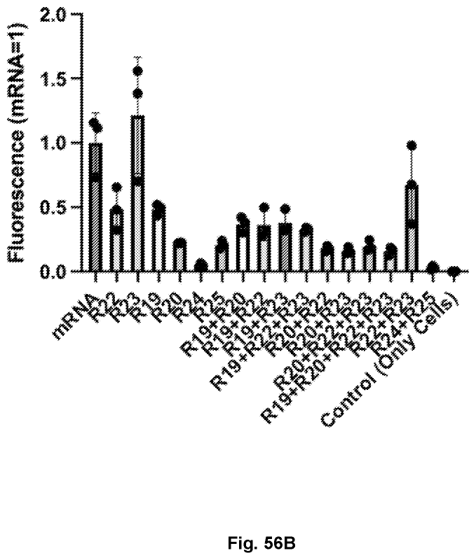

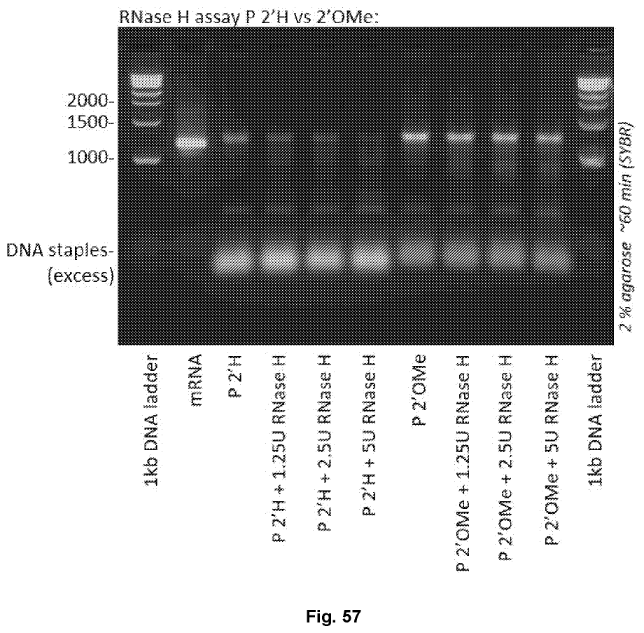

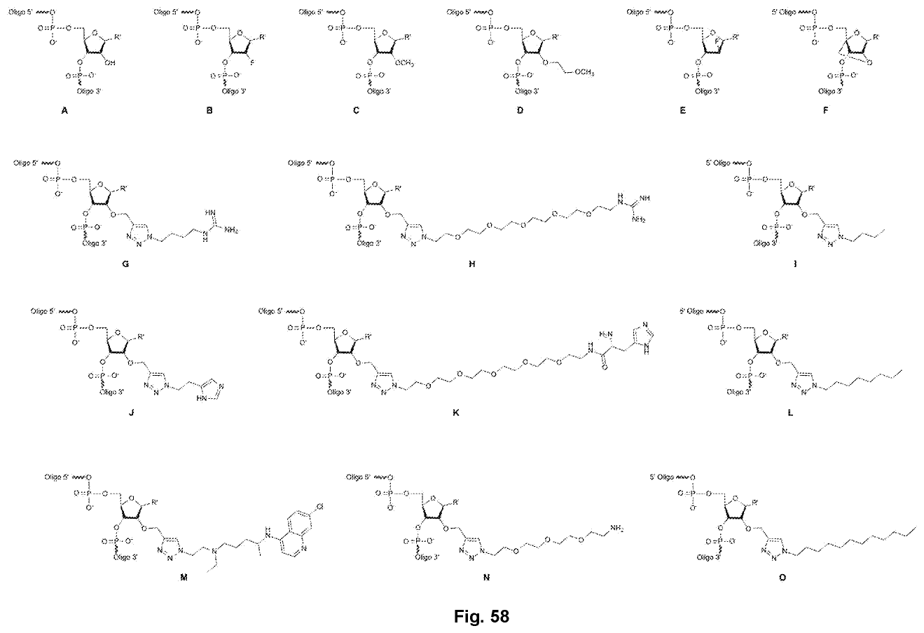

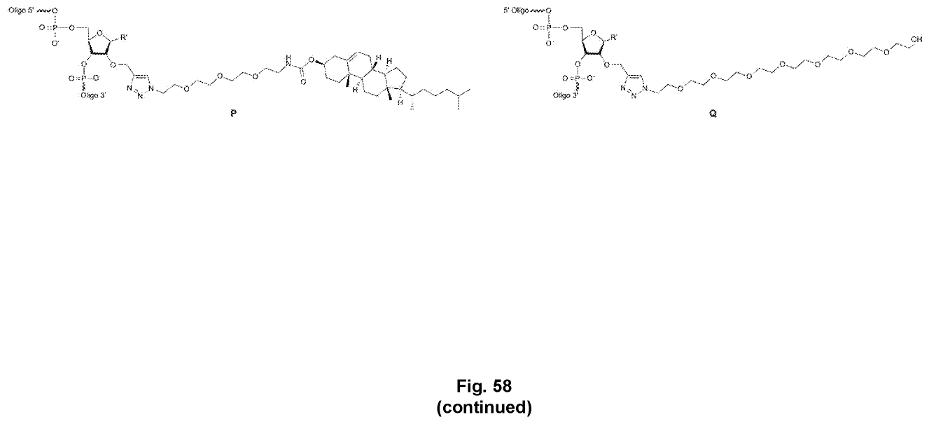

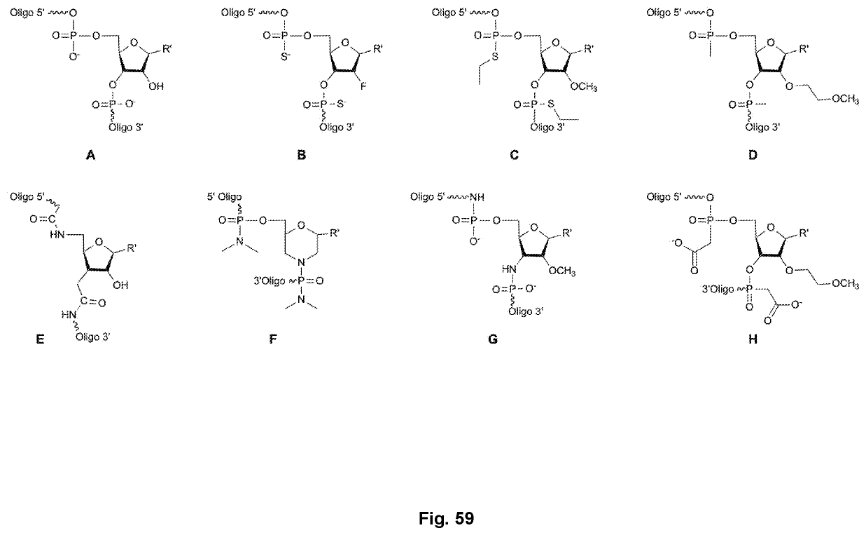

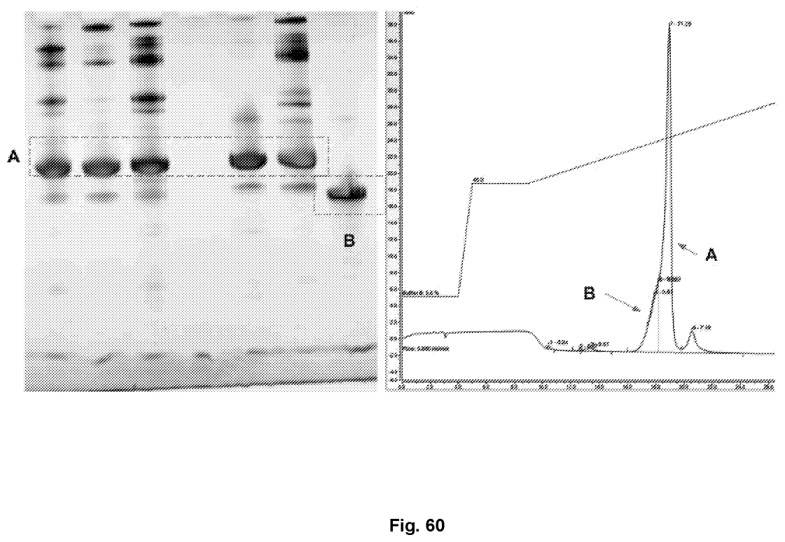

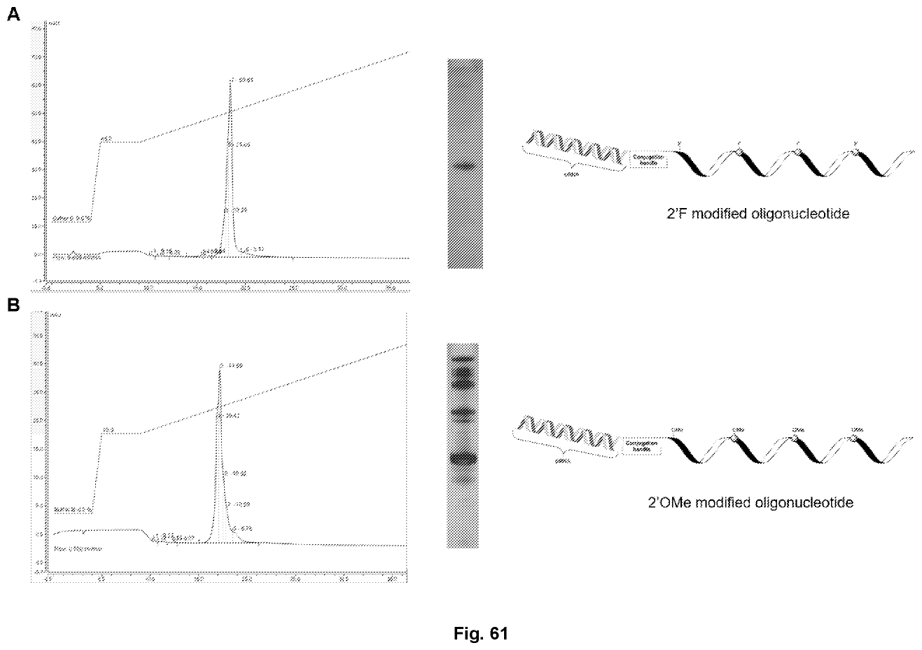

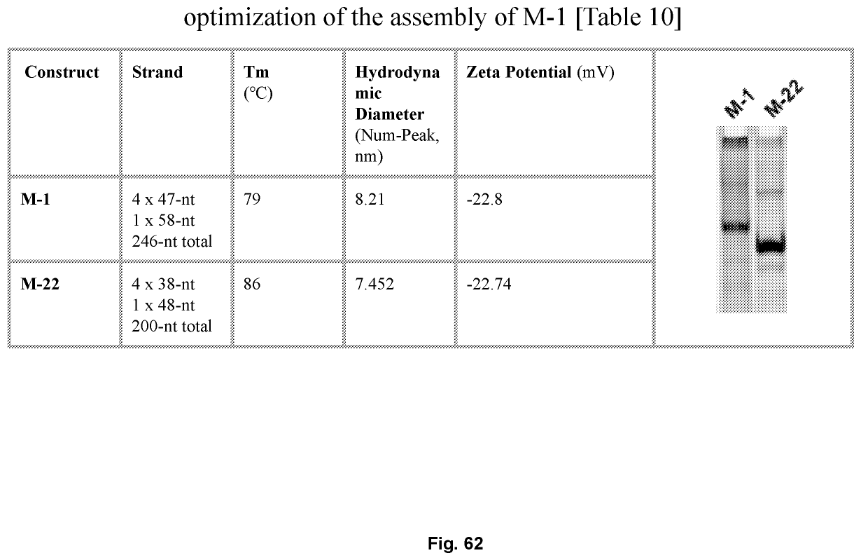

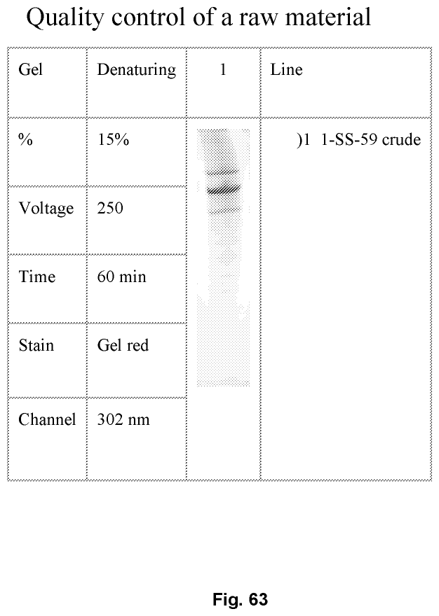

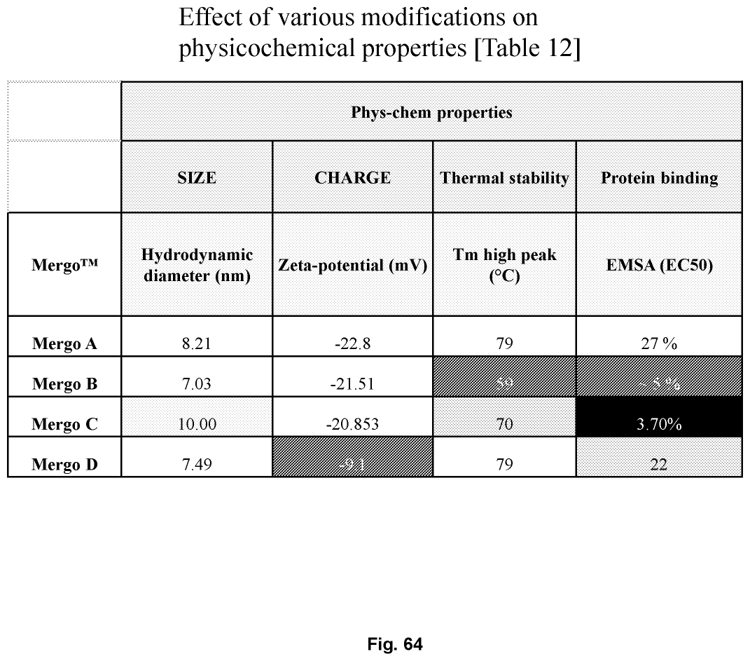

Incorporation of the modification post-synthetically may come with some challenges and full conversion may be difficult to achieve. To avoid these potential issues, the PK/PD modulating modification may also be incorporated directly onto the functional monomer. The central core may be a tertiary amine or serinol-based. Proposed synthetic routes are given in FIG. 19 (norbornene modifier), FIG. 20 (PK/PD modulating modifier, amine core) and FIG. 21 (PK/PD modulating modifier, serinol core). To generate the oligophosphate with the amine core, the desired modification must have a primary amine that can be reacted with a bromoalcohol. The bromoalcohol can include, but is not limited to, 2-bromoethanol, 3-bromo-1-propanol, 4-bromo-1-butanol, 5-bromo-1-pentanol, 6-bromo-1-hexanol, 7-bromo-1-heptanol. Double addition to the amine then affords the monomer that can be DMTr protected and phosphitylated.

The oligophosphate may be conjugated to the 5′ end of an oligonucleotide via a click handle that is incorporated within the oligophosphate chain. This might be at either terminal position or any given internal position.

RNA therapeutics may be conjugated with highly modified oligophosphate strands, as outlined in FIG. 17. Both ASO and siRNA molecules can be conjugated at one terminus, or at both. The modified oligophosphate strand may form a tertiary structure. The oligophosphate may also be modified in such a way that the chain can fold in on itself and form covalent linkages.

Additionally, direct modification of the 2′ position of nucleosides may also be carried out to form a more naturally derived modifying polymer. For example, highly modified XNA strand C-1.4 was coupled to S-1.3, whereby the long strand (C-1.4) was modified with 2′OMe throughout and the PPIB sense strand (S-1.3) was modified with 2′OMe and PTO. The purification of these species is non-trivial and required extensive IEX method development. Heavily modified conjugated strands, particularly strands that have a high loading of 2′OMe, tend to co-elute with the starting material. (FIG. 60). It is possible to drive the conversion to the product, however, higher order products tend to form when a large excess of the short strand (i.e., S-1.3) is used. Analytically pure material can be obtained by taking the latest eluting fractions. However, this leads to a dramatic decrease in yield. Arginine and histidine-modified oligonucleotides were also conjugated using this methodology. 2′F modified oligonucleotides have also been conjugated to a PPIB siRNA molecule. This can act as a standalone conjugate or be incorporated into higher order constructs. A comparison of the analytical data between 2′F and 2′OMe conjugates is given in FIG. 61.

### Example 12—RNA Nanoparticles with a Xeno Nucleic Acid (XNA) Backbone

The nucleic acid nanoparticles described in this invention may incorporate xeno nucleic acids (XNAs) in the backbone of the component oligonucleotides. XNAs are chemically modified nucleic acid analogues, whereby the sugar component is either modified or replaced. 2′F and 2′OMe modified nucleosides are classed as XNAs and these are incorporated into many aspects of the current invention. Additional XNA modifications that may be incorporated into the nucleic acid nanoparticles include, but are not limited to, FNA, FANA, 2′,4′-diFANA, 2′OMe, MOE, 2S-MOP, LNA, AmNa, R-5′-Me-LNA, S-5′Me-LNA, methylene cLNA, N-MeO-amino BNA, 2′4′-BNA^{NC}, N-Me-aminooxy BNA, 2′4′-BNAcoc, 2′4′-BNAcocPh, tricyclo DNA, HNA, FHNA, S-cEt, s-cMOE, CeNA, F-CeNA, Me-SRNA, MOE-SRNA, TNA, UMA, WNA, GuNA.

### Example 13—Atomic Force Microscopy (AFM) of Nucleic Acid Nanoparticles

Nucleic acid nanoparticles were imaged by Atomic Force Microscopy using a Bruker Dimension FastScan XR using Bruker FastScan D cantilevers. To immobilize the Mergo, 6 μL of sample at 10 μM were added to a freshly cleaved mica disk with 30 μl of NiCl₂ buffer. After 30 min of incubation, excess sample was removed by performing three washes with NiCl₂ buffer. Imaging was performed in 60 μL of NiCl₂ buffer. The AFM images are shown in FIG. 23.

### Example 14—RNA Nanoparticle Formation and Characterization with PK/PD Modulating Modifications

### Assembly of Modified Nucleic Acid Nanoparticle Constructs

Stoichiometric amounts of the different strands (5 to 7) were combined in the assembly buffer (PBS+MgCl₂ (2 mM)), with a final construct concentration of 10 μM. The strands were annealed to each other at 95° C. for 5 min then slowly cooled down to 4° C. (2.5° C./min), using a PCR thermocycler. The scaffold was then analyzed by native polyacrylamide gel electrophoresis (PAGE). For PAGE, the assembled scaffold was electrophoresed on native PAGE (6%) in 1×TBMg (890 mM Tris Borate+20 mM Mg(OAc)₂, pH=8.3) at a constant voltage of 100 V. Gel bands were visualized using Cy3, then stained with GelRed™. 10 pmol of structures was loaded. 2 μL of glycerin (70% in H₂O) was added to samples before loading. The resultant native PAGE is shown in FIG. 24.

### Zeta-Potential and Dynamic Light Scattering (DLS)

Particle size (hydrodynamic diameter, d) and surface charge (zeta potential) were analyzed on a Zetasizer Ultra instrument (Malvern, UK). We used the diffusion barrier technique to measure 20 μL of sample, in a DTS1070 (Malvern, UK) measurement cuvette. The measurement was performed at 37° C. The analyzed material was set to ‘Proteins’, which reliably measures the electrophoretic mobility of proteins or other fragile samples. Attenuation and voltage selection was set to automatic and equilibration time to 90 sec. The monomodal analysis mode was used for data analysis. The results of this analysis are shown in FIG. 25.

### UV-Vis Absorption Spectroscopy

Experiments were carried out using a Cary3500 UV-vis spectrophotometer, in quartz cuvettes (rectangular, 10 mm, 70 μL). Concentration of Mergos was 0.5 μM in assembly buffer. 70 μL of silicon oil was pipetted on the top of Mergo solution to prevent evaporation. Absorbance was measured at 260 nm and 375 nm and detected in increments of 1° C. from 15° C. to 95° C. Heating and cooling was done at a rate of 2.5° C./min. Melting temperatures are calculated by taking the temperatures corresponding to the derivative maxima of the curves obtained. These results are shown in FIG. 26.

### Physicochemical Properties: Electrophoretic Mobility Shift Assays

EMSA. To each 1 μL aliquot of 10 μM assembled Mergo (prepared as described previously), was added the desired number of equivalents of human serum or cerebrospinal fluid diluted in 1×PBS. Solutions were incubated at 37° C. for 30 minutes before adding 2 μL of 70% (v/v) glycerine solution to aid PAGE loading. Samples were analyzed by 6% native PAGE, ran for 1 h at 100 V at room temperature (FIG. 27).

Gel analysis. PAGE gels were imaged under the Cy3 channel (532 nm) to see the fluorophore-labelled products and after that, under the GelRed channel (staining with GelRed® 1× solution, for approximately 10 minutes). Shifts to a lower mobility reveals binding to serum proteins and formation of a so-called ‘protein corona’.

Band quantification. The intensity of individual bands was quantified using Image Lab software. Boxes were manually drawn around each band of interest and intensity was extracted for each one. The intensity of the bands was normalized to intensity of the band at t=0 h. Data was then analyzed using GraphPad Prism software. EC₅₀ shift equation was used to determine EC50 (Y=Bottom+(Top−Bottom)/(1+(EC/X)\hat{ }HillSlope), where X is the concentration, Y is the band intensity).

### Physicochemical Properties: Summary

Table 12 as provided in FIG. 64 shows the effect of various modifications on physicochemical properties. Mergo A does not carry any modifications. Mergo B, C and D are modified with different types of modifications. The size is retained, but the other parameters can be altered (charge, thermal stability, protein binding). The largest changes are indicated with a darker color.

### Physicochemical Data: Serum Stability

Snake Venom Phosphodiesterase stability assays (FIG. 28). Snake venom phosphodiesterase I (SV) from Crotalus adamanteus was purchased from Sigma-Aldrich. SVP was prepared as a stock of 2000 mU/mL, aliquoted into 1 mL Eppendorf tubes and stored at −20° C. All reactions were performed in 0.2 mL PCR tubes. Mergos™ were assembled as described above. 0.1 nmol of each Mergo were mixed with appropriate number of units of SV I in 1×PBS. Samples were incubated at 37° C. and 10 μL aliquots taken at different time points. 3 μL of 70% glycerol were added to each aliquot before loading the native PAGE and 8 μL of sample was loaded on 6% native PAGE, ran for 1 h at 100 V at room temperature.

RNase III stability experiments (FIG. 28). 0.1 nmol of each Mergo™ system was mixed with the appropriate number of units of RNase III E. coli (Cambridge Bioscience) and 10× RNase III buffer was added to a final concentration of 1× following the supplier instructions. The mixture was incubated at 37° C. and 10 μL aliquots were taken at specific times. 3 μL of 70% glycerol were added to each aliquot before loading the PAGE and samples were analyzed by 6% native PAGE, ran for 1 h at 100 V at room temperature.

Gel analysis. PAGE were imaged under Cy3 channel (532 nm) to see the fluorophore-labelled products and after that, under the GelRed channel (staining with GelRed 1× solution, for approximately 10 minutes).

Band quantification. The intensity of individual bands was quantified using Image Lab and/or ImageJ software. Boxes were manually drawn around each band of interest and intensity was extracted for each one. In stability experiments, background intensity was considered. The intensity of the bands was normalized to intensity of the band at t=0 h. Data was analyzed using GraphPad Prism software. First-order decay kinetics were assumed to calculate half-life.

### Example 15—Covalently Linked siRNA Cargo Molecules Retain Silencing Activity

Transfections of human A549 lung carcinoma cells were performed either as forward transfections with Lipofectamine 2000 (11668027, Invitrogen, Thermo Fisher Scientific) (where indicated) in 24-well plates or as reverse transfections in 96-well plates using Lipofectamine RNAiMAX (13778150, Invitrogen, Thermo Fisher Scientific) as transfection reagent. The siGENOME RISC-Free Control (D-001220-01-05, Dharmacon) was used as a non-targeting transfection control (NTC).

For forward transfections, A549 cells in logarithmic growth phase were plated at 5,000 cells/well in a 24-well plate on the day prior to transfection. One day later, the cell culture medium was aspirated and replaced by 200 μL of fresh DMEM/F12 medium (11330032, Gibco, Thermo Fisher Scientific) supplemented with 10% (v/v) heat-inactivated fetal bovine serum (F9665, Sigma Aldrich, Merck) and 1% (v/v) Penicillin-Streptomycin solution (15140122, Gibco, 15140122). Mergo constructs or free siRNA were diluted in Opti-MEM™ I Reduced Serum Medium (31985070, Gibco, Thermo Fisher Scientific) to 6× the final concentration. Likewise, Lipofectamine 2000 reagent was diluted 1:100 in Opti-MEM and incubated for 5 min at room temperature. Equal volumes of RNA dilution and lipofectamine dilutions were then combined and, after an incubation period of 20 minutes at room temperature, 100 μL of oligomer-Lipofectamine 2000 complexes were added to each well containing cells and medium. The cells were incubated at 37° C. in a CO₂ incubator for 48 hours, washed with cold PBS and the plate frozen at −80° C. Total RNA was extracted using RNeasy Plus Mini kits (74136, Qiagen), reverse transcribed with random primers using Superscript III reverse transcriptase (18080093, Invitrogen, Thermo Fisher Scientific) and the cDNA was then subjected to real-time PCR on a Quantstudio 5 thermal cycler (Applied Biosystems, Thermo Fisher Scientific) using PowerUp SYBR Green Master Mix (A25742, Applied Biosystems, Thermo Fisher Scientific).

For reverse transfections, 5 μL of RNA diluted in OptiMEM to 20× the final concentration was combined with 0.2 μL of Lipofectamine RNAiMAX and 14.8 μL Opti-MEM in each well of a 96-well plate. After a 15-minute incubation at room temperature, 80 μL of A549 cells in DMEM/F12 supplemented with 10% FBS without antibiotics were added to reach a final cell density of 4,000 cells per well. Approximately 48 hours later, the cells were either subjected to RNA extraction as described above or processed using the FastLane Cell SYBR® Green Kit (216213, Qiagen) or the Luna® Cell Ready One-Step RT-qPCR Kit (E3030S, New England Biolabs) as indicated, according to manufacturer's instructions. One-step RT-qPCR was performed on a Quantstudio 5 thermal cycler or a qTOWER³ 84 instrument (Analytik Jena). Primer sequences were hPPIB forward (5′-GTTTGGCAAAGTTCTAGAGG-3′), hPPIB reverse (5′-ACATCCTTCAGGGGTTTATC-3′), hRPLP0 reverse (5′-CTTCGCTGGCTCCCACTT-3′) and hRPLP0 forward (5′-CCATTGAAATCCTGAGTGATGTG-3′).

All transfections and qPCR runs were performed in technical triplicate in two independent experiments unless otherwise stated. The data is shown in FIG. 29.

### Example 16—Increased Construct Loading

Using Lipofectamine RNAiMAX (13778150, Thermo Fisher Scientific) as a transfection reagent, human A549 lung carcinoma cells were reverse transfected with Mergos loaded with one to two mono- or di-siRNAs at a cell density of 4,000 cells per well in a 96-well plate according to manufacturer's instructions. Mergos were transfected at equal concentration (0.1 nM) independent of the number of siRNA molecules attached. For comparison, unloaded mono- or di-siRNAs were transfected at concentrations equivalent to 1× (0.1 nM), 2× (0.2 nM) and 4× (0.4 nM) the base concentration. siGENOME RISC-Free Control (D-001220-01-05, Dharmacon) was used as a non-targeting transfection control (NTC). After 48 hours, the cells were washed with cold PBS and the plate frozen at −80° C. Total RNA was extracted using RNeasy Plus Mini kits (74136, Qiagen), reverse transcribed with random primers using Superscript III reverse transcriptase (18080093, Invitrogen, Thermo Fisher Scientific) and the cDNA was then subjected to real-time PCR on a Quantstudio 5 thermal cycler (Applied Biosystems, Thermo Fisher Scientific) using PowerUp SYBR Green Master Mix (A25742, Applied Biosystems, Thermo Fisher Scientific). For statistical analysis, 1-way repeated measures ANOVA was performed with Tukey's post-hoc test.

The results of this experiment are shown in FIG. 30.

### Example 17—Gene Silencing Data from Constructs Loaded with Endosomal Escape Peptides

FIG. 31 shows gene silencing data of nucleic acid nanoparticles that were loaded with peptides that were developed with the potential to mediate endosomal escape. Transfection of human MDA-MB-231 breast cancer cells and 2-step RT-qPCR was performed as described above (Example 15—Covalently linked siRNA cargo molecules retain silencing activity) using Lipofectamine 2000 as transfection reagent. Cells were plated at 5,000 cells per well in 24-well plates and Mergos were transfected at a final concentration of 20 nM for 48 hours. For free uptake experiments, cells were incubated with 200 nM of RNA nanoconstructs in the absence of transfection reagent.

The GFP-GAL9 assay was also utilized to determine endosomal escape (FIG. 32). (M. J. Munson, G. O'Driscoll, A. M. Silva, E. Lázaro-Ibinez, A. Gallud, J. T. Wilson, A. Collen, E. K. Esbjorner, A. Sabirsh, A high-throughput Galectin-9 imaging assay for quantifying nanoparticle uptake, endosomal escape and functional RNA delivery, Commun. Biol. 4 (2021) 211).

Stable Hela cells expressing GFP-GAL9 were generated by lentiviral transduction. Cells were seeded at 20×10³ cells/well (96-well) and incubated with lentivirus packaged with the GFP-GAL9-355 vector as per manufacturer's instructions. Cells were incubated for 48 h before the addition of 1 g/ml puromycin to select for stably integrated cells. The generated Hela cells stably expressing GFP-GAL9 were seeded at 7.5×10³ cells/well (96-well) and incubated with 200 nM of each Mergo (SQ) or 75 μM chloroquine. At the end of each experiment (0, 1, 4, 8 and 24 h) cells were washed with phosphate buffered saline (PBS) and fixed with 4% paraformaldehyde (PFA) for 10 min at room temperature (RT). After fixation, cells were washed with PBS, permeabilized with 0.1% Triton-X for 10 min, washed with PBS and incubated with 2% BSA in PBS for 30 min. Nuclei were stained with 4′,6-diamidino-2-phenylindole (DAPI), washed with PBS, mounted on microscope slides and imaged using a confocal microscope. Images were processed on Columbus software. Data were plotted on GraphPad Prism 9.3.1.

### Example 18—Biodistribution and Gene Silencing of Nucleic Acid Nanostructures

All experimental studies involving animals were approved by the UK Home Office. Female BALB/c mice (weight: approximately 20 g, 8-12 weeks old) received two tail vein injections of 200 μL Mergo at 10 ml/kg (day 0 and day 3). Each injection corresponds to 2 nmol of Mergo at a concentration of 10 μM. Mergos carry a Cy3 fluorescent dye. Vehicle injections were used as negative control and state of the art Lipid Nanoparticles (LNP) were used as comparison. Animals were group housed as appropriate in the animal facility and maintained under a 12 h light/dark cycle with free access to food and water, where temperature and humidity were controlled according to Home Office regulations.

On day 7, the animals were sacrificed by cervical dislocation and the liver, kidney, spleen, pancreas, lung and heart were excised and imaged for 5 sec, on an Azure biosystems c600. The imager was set at the Cy3 filter with a 127 nm resolution. The fluorescent intensity of the organs was analyzed on ImageJ Fiji. As the Cy3 intensity depends on the chemical modifications of the Mergo, the data were corrected to the corresponding Cy3 intensity. Organ autofluorescence was assessed by the control—vehicle injected animals. Mergo fluorescence was subsequently normalized to the control vehicle.

Organs stored in RNAlater (Sigma, R0901) were pierced using 2 mm punches to assess the gene silencing by mRNA quantification. The pierced tissues were lysed using QuantiGene Sample Processing Kit, Tissues (Invitrogen, QS0106) according to the manufacturer's instructions using a Tissue Lyzer II (Qiagen). mRNA was detected according to the Quantigene 2.0 protocol using the following probe sets: mouse HPRT (SB-15463), mouse PPIB (SB-10002). All data were plotted on GraphPad Prism 9.3.1. The data from these experiments is shown in FIG. 33-35. Constructs with modifications outlined in FIG. 1 were used (i.e., PK/PD modulating modifications dispersed throughout the 2′ position of component nucleotides).

### Example 19—Toxicity Studies on Modified Constructs

Blood, typically 100 μl, was collected into sodium heparin, from mice via the tail vein, at 2 h post injection and plasma prepared by centrifugation (14,000 rpm, 4° C., 5 min.). Cytokine levels, using manufactured bespoke kits, were determined by MAGPIX Luminex system. Clinical chemistry of plasma ALT/AST was completed using a Beckman Coulter instrument. Mice were weighed prior to treatment and on day 7 post initiation of the treatment. The data from these experiments are shown in FIG. 36.

### Example 20—Synthesis of a Nucleic Acid Nanoparticle Comprised of L-RNA

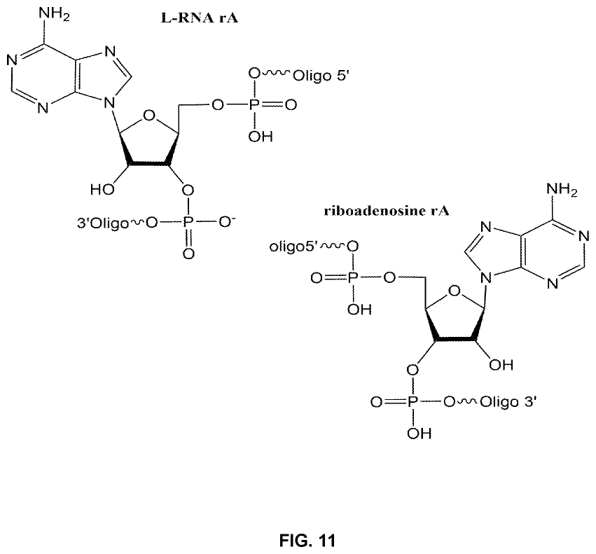

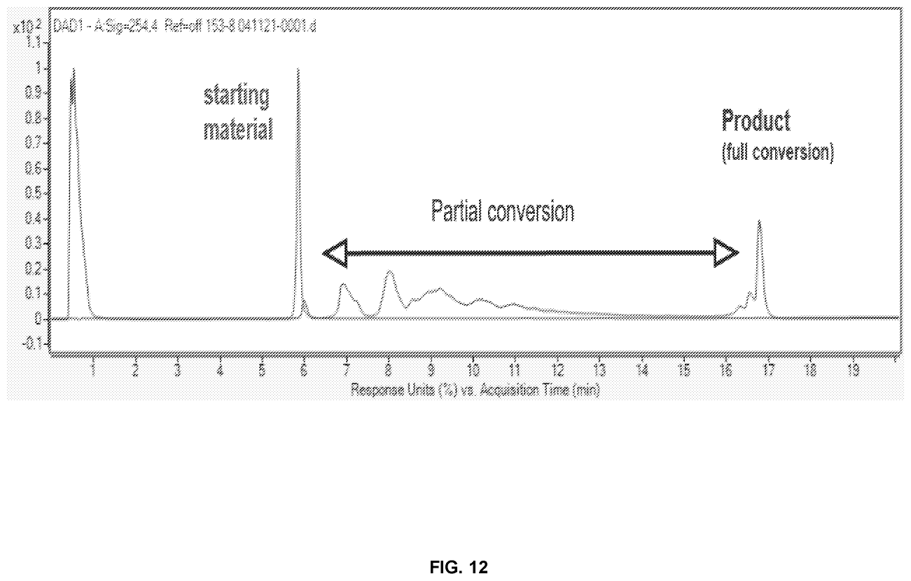

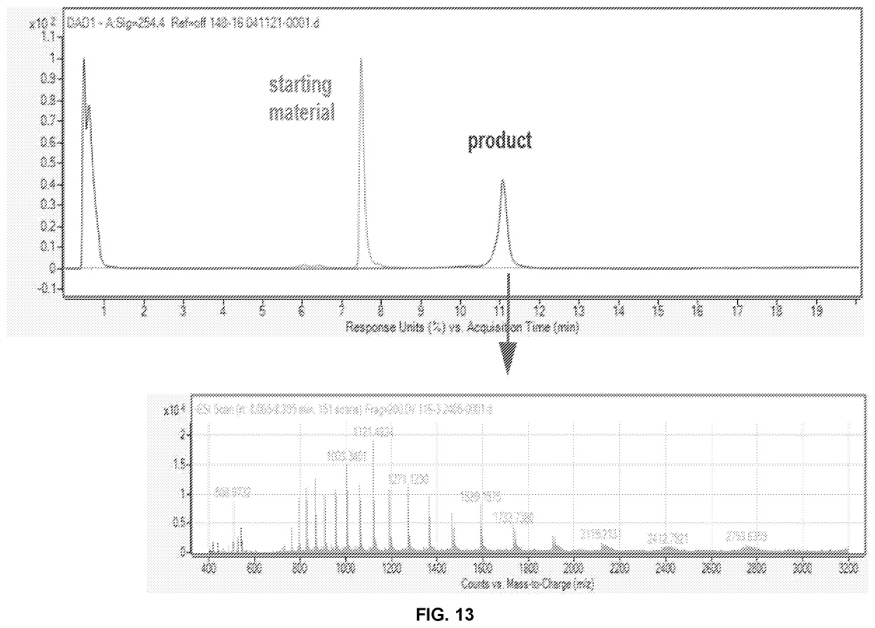

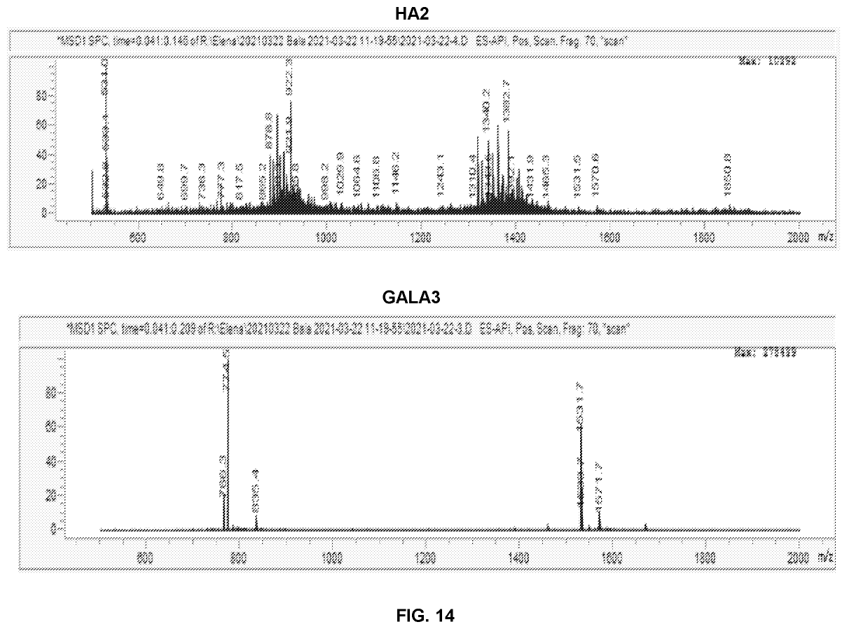

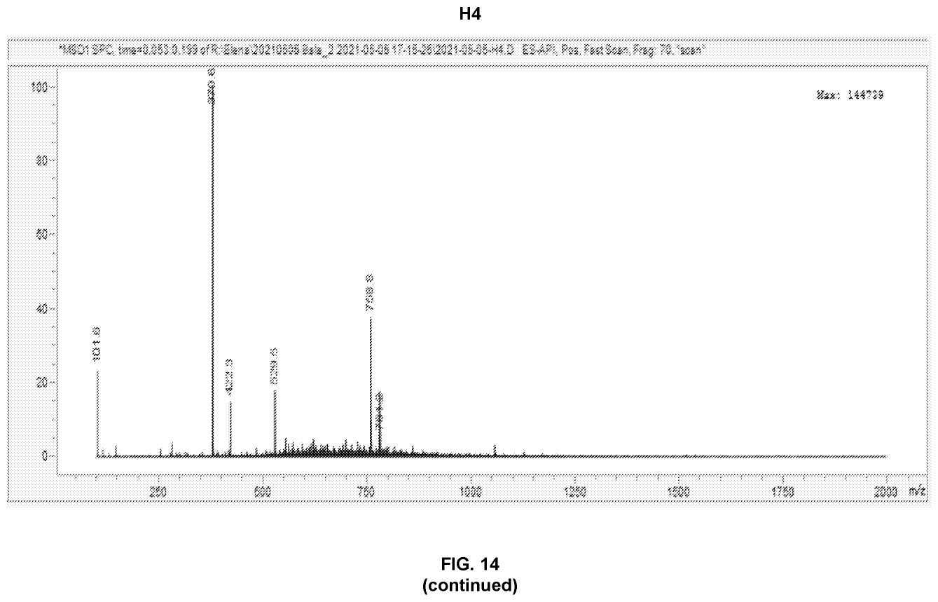

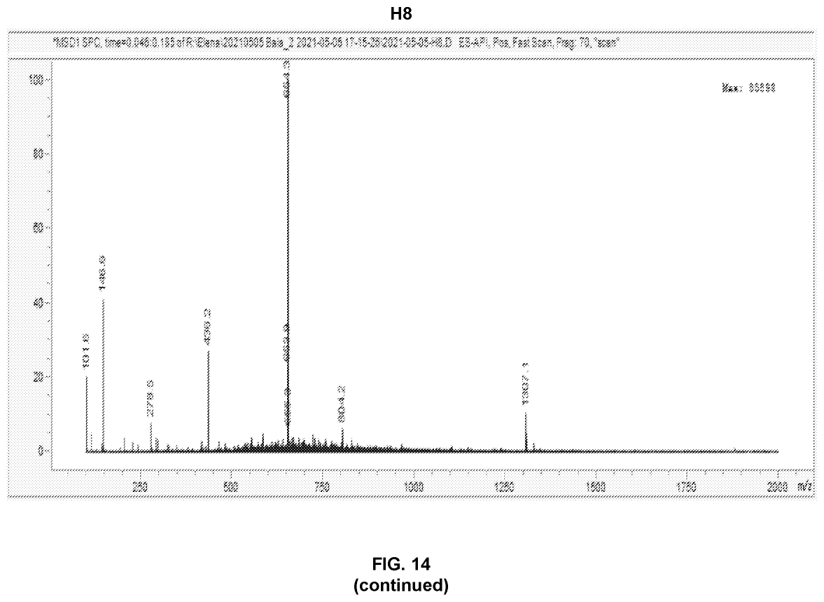

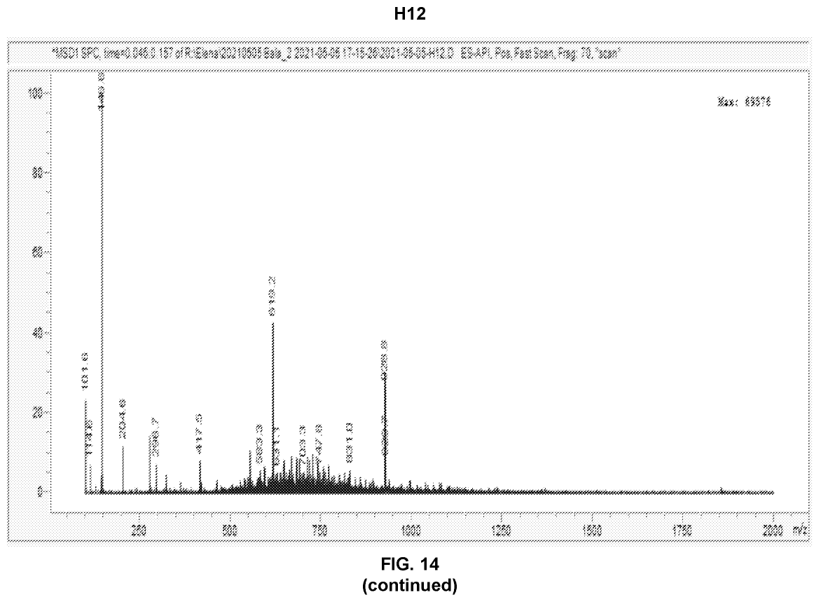

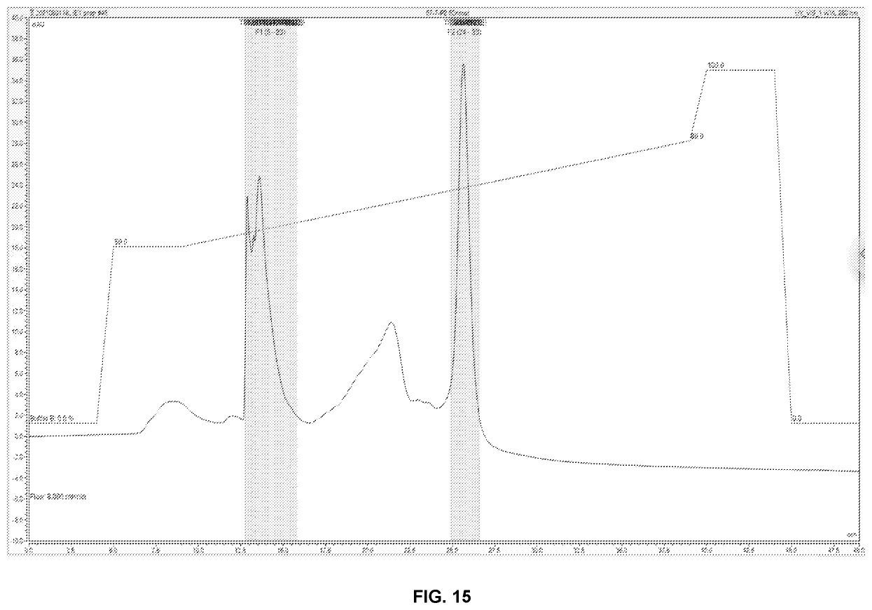

The core nanostructure will comprise L-RNA as shown in FIG. 11 with two siRNA's attached by IEDDA. The L-RNA constructs will be manufactured by solid phase oligonucleotide synthesis using commercially available L-RNA nucleobases, (e.g., phosphoramidites). Strands can be purified using either HPLC or PAGE-based methods. Following successful isolation of the purified oligonucleotides, assemblies can be performed by mixing strands in an ionic buffer at equimolar ratio, and using a thermal anneal protocol (generally strands mixture held at 95° C. for 5 minutes then slowly cooled down to 15° C. (85° C. for 2 minutes, 75° C. for 2 minutes, 65° C. for 2 minutes, 55° C. for 2 minutes, 45° C. for 2 minutes, 35° C. for 2 minutes, 25° C. for 2 minutes, 15° C. for 2 minutes)). Assemblies will be verified using DLS and native gel electrophoresis (6. PAGE with TBMg (890 mM Tris Notate+20 mM Mg(OAc)₂, pH=8.3) buffer). Enzymatic stability of the construct can be measured by incubating the constructs in relevant physiological buffers (e.g., blood, serum, from human, mouse). Time points are collected, and RNA constructs are isolated from the mixture by Proteinase K treatments or phenol-chloroform extraction. Intact constructs can be measured using LC-MS or PAGE. In vivo properties and distribution can be assessed using fluorescently labelled constructs (such as Cy7).

### Example 21—RNA Origami Parameters

To study the proof of concept of the RNA origami, a commercially available mRNA encoding for eGFP was bought from Trilink to be used as scaffold mRNA. As the single-stranded staple strands offer a lot of possibilities in terms of composition and complementarity regions, the decision was made to study their fundamental properties. Antisense oligos were designed (Sequences are given in Table 13) complementary to the open reading frame of the eGFP mRNA. The same sequence as unmodified DNA, PS-DNA, 2′OMe-DNA and unmodified RNA (FIG. 55) was compared. Unmodified DNA and PS-DNA inhibit translation (most probably non giving protection against nucleases), whereas RNA seems to be the best suited to be used as single-stranded staple species.

600 fmol of mRNA were assembled with 3 pmol of antisense oligos in 1 μM PBS. DNA oligo, thiophosphorylated (ps) DNA oligo, RNA oligo and 2′OMe-DNA oligo was compared.

TABLE 13
antisense strands used for binding to the ORF region to assess the suitability of
differently modified oligonucleotides.
Antisense strands for binding to the ORF region
DNA CTTGTCGGCCATGATATAGACGTTGTG
2′OMe CTmUGTCmGGCCmATGAmUATAmGACGmUTGTmG
PS C*T*T*G*T*C*G*G*C*C*A*T*G*A*T*A*T*A*G*A*C*G*T*T*G*T*G
RNA rCrUrUrGrUrCrGrGrCrCrArUrGrArUrArUrArGrArCrGrUrUrGrUrG

The mRNA was incubated with the antisense species for 5 min at 80° C., followed by a 30 min temperature gradient 80 to 25° C. The assembly was then transfected in A549 cells and fluorescence was measured 22 h after transfection on Tecan Infinite 200 Pro. The next step was to assess how the introduction of folding on the scaffold mRNA through RNA and/or 2′OMe-DNA single-stranded staples influences the translation efficiency. Translation should be more inhibited when the folding is induced on regulatory elements present in the 5′ and/or 3′ untranslated regions and less inhibited when folding is occurring in the open reading frame. Based on FIG. 55, it was assumed that mRNA folding induced by RNA staples should be more permissive and lead to higher translation levels compared to 2′OMe-DNA staples.

To test this, 600 fmol of mRNA were assembled with 3 pmol of 2′OMe-DNA staple strands in 1 μM PBS. The mRNA was incubated with the antisense species for 5 min at 80° C., followed by a 30 min temperature gradient 80 to 25° C. The assembly was then transfected in A549 cells and fluorescence was measured 22 h after transfection on Tecan Infinite 200 Pro.

Modulation of translation efficiency based on the hybridized region of the mRNA scaffold both on the open reading frame and 3′UTR is observed (FIG. 56). Interestingly when a staple with high translational outcome is combined with a staple with lower translational outcome, the resulting value falls within an average range between the two. These results give us the basis to design more efficient iterations of staple testing.

### RNase H Stability Assay:

A semi-assembled version of the 10HB_rectangle (variant P with staples R19-25) was tested for RNase H resistance. For this purpose, 2′OMe mixmers with a gap space of DNA nucleotides between the 2′OMe modifications (Table 14) were acquired. 2′OMe modified and non-modified DNA staple strands (20 pmol each strand) were added to separate tubes eGFP mRNA (800 fmol). The mixtures were assembled via a 1 h gradient from 75 to 20° C. After assembly the volumes of the 2′OMe modified and the non-modified assay were quartered. To the aliquots 0, 1.25, 2.5, and 5 U of RNAse H were added together with RNase H buffer. The mixtures were incubated at 25° C. for 30 min and cleavage was analyzed via native 2% agarose gel (FIG. 57). The 2′OMe modified assembly demonstrated a much higher sustainability against nuclease than the unmodified assembly.

TABLE 14
2′OMe modified (mX) versions of 10HB_rectangle staples used for RNase H assay:

R19-2′OMe
GCmUTCTmCGTCmACGAmACTCmCAGCmAGGAmUTCCmUACT
R20-2′OMe
GmGGTGmCTCACTmCGTCmCATGmCCGAmGAGTACmAGGTmG
R21-2′OMe
mCGGGmGCCGmUGCAmGCTTmAATTmAAGCmGGAGmAAGGmCAAGmCCCCmGCA
R22-2′OMe
GmCGGCmGGTTmGGGGmUCTTmUGCTmCAGCmGATGmUTG
R23-2′OMe
TTmGTACmAGmGGTAmGTGGmUTGTmCGGGGGmUAGTmGG
R24-2′0Me
mCAGGmCTTTmATTCmAAAGmACCAmAGAGmGTmGATCmCCG
R25-2′OMe
CAAmGGGAmGAGAmAGAAmGGGCmATGGmCCCCmGCTTmAC

### Example 22—RNA Origami Design

To fold an origami applying a mRNA as a scaffold and short DNAs as staple strands, an 8-9-8-8 crossover strand layout for the design of the 2D rectangular origami 10HB_rectangle was used. This is used to compensate for the different helix geometry that comes with the change from DNA-DNA duplex (B-helix, 10.5 bp/turn) to RNA-DNA (A-helix, 11.0 bp/turn) (see FIG. 45). For the 6HB_tube and 10HB_block a crossover strand layout that is commonly used for DNA origami tubes and block structures in a 120° honeycomb model was utilized. This was shown in the literature is also applicable for RNA-DNA origamis (see FIG. 46 and FIG. 47).

The chosen sequence for the scaffold was an mRNA that codes for an enhanced green fluorescence protein (eGFP) with the 5′-UTR of HIV envelope glycoprotein and 3′-UTR of hemoglobin alpha-2 (see Table 15). The mRNA was purchased from TriLink Biotechnologies. The sequence for the DNA staples that were used to fold the different structures out of the eGFP mRNA are shown in Table 15. The individual DNA staple strands were purchased from IDT. For effective translation, the mRNA was 5′-capped (cap1) and poly(A)-tailed. The designs were successfully tested in oxDNA on their molecular dynamic parameters (see FIG. 48, FIG. 49, FIG. 50).

TABLE 15
example of eGFP mRNA scaffold and associated DNA staples to form
10HB_rectangle, 6HB_tube, and 10HB_block
eGFP mRNA scaffold (with 5′-cap1)
>EGFP mRNA
AAGAGAGAAA AGAAGAGTAA GAAGAAATAT AAGAGCCACC ATGGTGAGCA AGGGCGAGGA
GCTGTTCACC GGGGTGGTGC CCATCCTGGT CGAGCTGGAC GGCGACGTAA ACGGCCACAA
GTTCAGCGTG TCCGGCGAGG GCGAGGGCGA TGCCACCTAC GGCAAGCTGA CCCTGAAGTT
CATCTGCACC ACCGGCAAGC TGCCCGTGCC CTGGCCCACC CTCGTGACCA CCCTGACCTA
CGGCGTGCAG TGCTTCAGCC GCTACCCCGA CCACATGAAG CAGCACGACT TCTTCAAGTC
CGCCATGCCC GAAGGCTACG TCCAGGAGCG CACCATCTTC TTCAAGGACG ACGGCAACTA
CAAGACCCGC GCCGAGGTGA AGTTCGAGGG CGACACCCTG GTGAACCGCA TCGAGCTGAA
GGGCATCGAC TTCAAGGAGG ACGGCAACAT CCTGGGGCAC AAGCTGGAGT ACAACTACAA
CAGCCACAAC GTCTATATCA TGGCCGACAA GCAGAAGAAC GGCATCAAGG TGAACTTCAA
GATCCGCCAC AACATCGAGG ACGGCAGCGT GCAGCTCGCC GACCACTACC AGCAGAACAC
CCCCATCGGC GACGGCCCCG TGCTGCTGCC CGACAACCAC TACCTGAGCA CCCAGTCCGC
CCTGAGCAAA GACCCCAACG AGAAGCGCGA TCACATGGTC CTGCTGGAGT TCGTGACCGC
CGCCGGGATC ACTCTCGGCA TGGACGAGCT GTACAAGTAA GCGGCCGCTT AATTAAGCTG
CCTTCTGCGG GGCTTGCCTT CTGGCCATGC CCTTCTTCTC TCCCTTGCAC CTGTACCTCT
TGGTCTTTGA ATAAAGCCTG AGTAGGAAGA AAAAAAAAAA AAAAAAAAAA AAAAAAAAAA
AAAAAAAAAA AAAAAAAAAA AAAAAAAAAA AAAAAAAAAA AAAAAAAAAA AAAAAAAAAA
AAAAAAAAAA AAAAAAAAAA AAAAAAAAA
DNA staples to form 10HB_rectangle
>EGFP-R01
TTTCTTCTTA CTCTTCTTTT CTCTCGGTCA GCT
>EGFP-R02
TCCTCGCCCT TGCTCACCAT GGTGGCTCGC CGG
>EGFP-R03
GATGGGCACC ACCCCGGGTC GCCGT
>EGFP-R04
TGCCGTAGGG GTGGTGCAGA TGAACTCTTG AAGA
>EGFP-R05
ACACGCTGAC ACGAGGGTGG GCCAGGTTCG GGCA
>EGFP-R06
CCAGCTCGAG CTGAAGCACT GCACGCCTTC ATGT
>EGFP-R07
AGCTTGCCTG GCATCGCCCT CGCCCTCTTA TA
>EGFP-R08
AGGGTGGTAC TTGTGGCCGT TTACTGAACA GC
>EGFP-R09
AGATGGTGCG GTCTTGTAGT TGCCGTGTTC TTCT
>EGFP-R10
TGGCGGACTC AGGGTGTCGC CCTCGAGTGG CTGT
>EGFP-R11
GGTCGGGGTG AAGTCGATGC CCTTCACAGG ATGT
>EGFP-R12
TCGGCGCGGC TCCTGGACGT AGCCGCACGG GC
>EGFP-R13
CGGTTCACTG AAGAAGTCGT GCTGCGTAGG TC
>EGFP-R14
GCTTGTCGGC TTGAAGTTCA CCTTGAGTGA TCGC
>EGFP-R15
TGTAGTTGTC TGCACGCTGC CGTCCTGGCG GACT
>EGFP-R16
TGCCGTCCTG ATGGGGGTGT TCTGCTCAGC AGCA
>EGFP-R17
TGGCGGATCC ATGATATAGA CGTTACTTCA CC
>EGFP-R18
TCGGCGAGAC TCCAGCTTGT GCCCGCTCGA TG
>EGFP-R19
GCTTCTCGTC ACGAACTCCA GCAGGATTCC TACT
>EGFP-R20
GGGTGCTCAC TCGTCCATGC CGAGAGTACA GGTG
>EGFP-R21
CGGGGCCGTG CAGCTTAATT AAGCGGAGAA GGCAAGCCCC GCA
>EGFP-R22
GCGGCGGTTG GGGTCTTTGC TCAGCGATGT TG
>EGFP-R23
TTGTACAGGG TAGTGGTTGT CGGGGGTAGT GG
>EGFP-R24
CAGGCTTTAT TCAAAGACCA AGAGGTGATC CCG
>EGFP-R25
CAAGGGAGAG AAGAAGGGCA TGGCCCCGCT TAC
DNA staples to form 6HB_tube
>EGFP-T01
TCGTGCTGCT TCATTCTCTC TCTTCCTACT CAGGCGGGGT GT
>EGFP-T02
GTGGTCGGGC ATGGCGGACT TGCTGCACTC TGCTGGTAGT GG
>EGFP-T03
GCTGCCGTCC TCGAGCCTTC GGGGTAGCGG CTGAATATTT CT
>EGFP-T04
TGTTGTGGGC CGTCGCCGAT GTTTATTCTC TTACTCTTCT TT
>EGFP-T05
AAAGACCAAG AGGTAGCACG GGCGGATCTT GAAGTGATGG TG
>EGFP-T06
ACGCCGTAGG TCAGTGCTCA CCAAGGGAGA GAAGAAGGTA GT
>EGFP-T07
ACAGGTGCAT GGTGGCTCTT AGCACTGCCG CTCCTGGACG TA
>EGFP-T08
GGTGGTCTCG TCCTTGAAGA ATCACCTTGG TTGTCGGGCA GC
>EGFP-T09
GATGCCGTTC TTCTGTTGCC GACGAGGGTG GGCCACGGTG AA
>EGFP-T10
GCTTGTCCGG ACTGGGTGCT CAGGGCATCA GCTCCTCGCC CT
>EGFP-T11
GGCCAGAAGG CAAGCTCAGG GGGCCATGAT ATAGACACCT CG
>EGFP-T12
GGCAGCTTGC CGGTCGACCA GGAAGGCAGC TTAATCGCTT CT
>EGFP-T13
CCCCGCAGAT GGGCACCACC CGGGCACGGC GCGGGTCTTG TA
>EGFP-T14
GGTGCAGTCG CCCTCGAACT TCGTTGTGCG TTGGGGTCTT TG
>EGFP-T15
GCTGTTGTAG TTGTCAGGGT GATGAACTTC AGGGTCGTTT AC
>EGFP-T16
ACTCCAGGGA CCATGTGATC GTAAGCGGGT CGCCGTCCAG CT
>EGFP-T17
CCGCTTACTT GTACTCCAGC ACTTGTGC
>EGFP-T18
CCCAGGACTT CAGCCCGTAG GTGGCATCGC CCTCG
>EGFP-T19
AGCTCGTGCT GAACTTGTGG CCAGCTTGTC GATGCGGTTC AC
>EGFP-T20
CCCTCGCCGG ACACCCATGC CGAGAGTG
>EGFP-T21
TTGAAGTCGA TGCCTGTTGC CCGGCGGTCA CGAAC
DNA staples to form 10HB_block
>EGFP-B01
GGTCACGAAC TCCATTTTTT TCTCTCTTGG TCAGCATGAA CT
>EGFP-B02
GACCATGGTT CACCTGCCGT TAGTTGCC
>EGFP-B03
GGTGCGCGGC GCGGGTCTTG TCTTCTGCTT CTTTT
>EGFP-B04
TTGTCGGTGG CGGATCTTGA ATGATCGC
>EGFP-B05
TCCTTGAAGA AGATGGTGCA GTTGCCGTAG GTGGCCTTAC TC
>EGFP-B06
TTTATTCATT TCTTATCGCC CGGCAGCTTG CCGGT
>EGFP-B07
CTTCCTACTC AGGCCGGCGG CGCTTCTCGT TGGGG
>EGFP-B08
ACGCTGCCGT CCTCTCTTTG CGCCGAGAGT GATCC
>EGFP-B09
GATGTTGCCA TGATTCACCT CTCCTGGACG TAGCC
>EGFP-B10
ACGAGGGTGG GCCATTCGGG CGTCGCCCTC GAACTATAGA CG
>EGFP-B11
GGGCACGTCG CCCTCGCCGG AATGGTGGTT GTGGC
>EGFP-B12
CTCTTATAAA GACCAAGAGG TCGTCCAT
>EGFP-B13
TTACTTGTAC AGCTACAGGT GGCTCACCCA CGCTG
>EGFP-B14
TCAGGGCGGA CTGGGAGCTG CTGTTGTACC AGGGT
>EGFP-B15
GAAGTCGCTC GATGCGGTTC AGTTGTACCG CCCTT
>EGFP-B16
TCCAGCTTGG TAGTGGTCGG CGTGCTCA
>EGFP-B17
ATGGCGGACT TGAAGGTGGT CAACTTGTGG CCGTTAGCTC CT
>EGFP-B18
GGTGAACTAC GTCGACGCCG TAGGTCAG
>EGFP-B19
CAAGGGAGAG AAGACGGCCG CGGTAGTGGT TGTCGGTTCT GC
>EGFP-B20
TGGGGGTGGC AGCAGCAGCT TAATTAAGAG GGCAT
>EGFP-B21
TGTGCCCCCT TCAGTGCTGC TTCATGTGGC ACTGC
>EGFP-B22
GG CTGAAGTCGG GGCTTGAAGT CGATGCCAGG ATG
>EGFP-B23
CCGTCCAGCT CGACATGGGC ATTGCCGTTC GCCGA
>EGFP-B24
CCACCCCGGC CAGAAGGCAA GGCAGAAGGC ACGGG

Assembly of mRNA Origami Constructs

600 fmol mRNA was assembled with 12 pmol DNA mix (either rectangle, tube or block mixture) in 10 mM Tris/HCl (pH 7.0) and 120 mM NaCl. The mixture was incubated for 5 min at 75° C., slowly (2° C./min) to 65° C. and very slowly (1° C./min) cooled down to 20° C. The samples were purified via 50 MWCO spin filter (4 wash steps, 10 min, 12,000 rcf) and the assembly was verified via band-shift assay on a 2% agarose gel (FIG. 51).

AFM Imaging of mRNA Origami (FIG. 52)

A freshly cleaved mica surface was preincubated with 10 mM NiOAc solution for 20 sec and washed three times with TE-buffer. The origami samples were highly diluted in TE-buffer. An aliquot of this diluted sample was put on the mica surface and incubated for 10 min prior to the imaging.

### Example 23—Circular mRNA

The chosen sequence for the scaffold is an mRNA that codes for an enhanced green fluorescence protein (eGFP) with the 5′-UTR of HIV envelope glycoprotein and 3′-UTR of hemoglobin alpha-2 (see Table 15). The mRNA was purchased from TriLink Biotechnologies. The sequence for the DNA staples that were used to fold the different structures out of the eGFP mRNA are shown in Table 16. The individual DNA staple strands were purchased from IDT. For effective translation, the mRNA was 5′-capped (cap1) and poly(A)-tailed.

For the ‘Handle_basic’ design (FIG. 53) the layout is based on the concept that, for the initiation of translation, 5′-end binding proteins interact with 3′-end binding proteins to form a circular structure that stabilizes the mRNA and facilitates initiation of translation. The aim therefore is to bind the sequences and/or linker sequences at the 5′ and 3′ end of the mRNA with one main staple strand. This staple strand can also bind a secondary and shorter staple strand that can be chemically modified. In addition, the ‘Handle_basic’ design will be tested with increasing number of intra-mRNA binding staples strands (example ‘Handle_2staples’).

Given that circular RNA has been shown to be more stable than linear mRNA, the delivery of circular mRNA scaffolds (mRNA with its ends covalently linked) with increasing number of staples (Circular_2staples, Circular_4staples) will also be tested (FIG. 53). 600 fmol of mRNA were assembled with 3 pmol of antisense oligos in 1 μM PBS. DNA oligo, thiophosphorylated (ps) DNA oligo, RNA oligo and 2′OMe-DNA oligo was compared.

TABLE 16
Staples used to fold structures out of the eGFP mRNA.
Staples to form circular mRNA
5′ TTCTCTCTTCTCTCTTGAGATCTCGCCACTCTAGTCCGGA 3′
5′ TCCGGACTAGAGTGGCGAGATCTC 3′
5′ CGTTGTGGCTGTTGTAGTTGTACTCTTCTTACTCTTC 3′
5′ TGCCGTTCTTCTGCTTGTCGGCCAT 3′
5′ TCGTGCTGCTTCATGTGGTCGGGGTAGCGGCTTAGTGGTCGGCGAGCTGCACGCTGCCGTCCTC 3′
5′ TTGTACAGCTCGTCCATGCCGAGAGTGATCCCGATGAACTTCAGGGTCAGCTTGCCGTAGGTGG 3′
5′ CAGGATGGGCACCACCTACAGGTGCAAGGGA 3′
5′ ACGTTGTGGCTGTTGTAGTTGCCGTCGTCCT 3′

### Example 23—RNA Nanoparticles with Increased Loading Via Branching Units and Poly-T

The branched siRNA was designed to increase the therapeutic loading capacity of the nucleic acid constructs and allow for the synthesis of more potent therapeutics in a more sustainable way (i.e., by reducing waste). This methodology will also enhance Mergo versatility and speed of development, i.e., ability to readily adapt to delivery of multiple, different cargo types, creating an intelligent delivery system that goes beyond the limitations of current standards. The designs utilised in this invention are outlined in FIG. 10.

The branching unit was incorporated into the oligonucleotide sequence using solid phase oligonucleotide synthesis. The branching unit allows attachment of more than one therapeutic moiety at a given location.

Alternatively, a double siRNA approach is also used which includes connections of two siRNA units with a linker poly thymidine (poly-T) in the form of a combinatorial chain (FIG. 6). This strategy is also helping with the above.

TABLE 17
sequences used in nucleic acid nanoparticles with increased therapeutic loading.

| ID | Sequence |
|---|---|
| Core strand + | 5′ GcAAuuAcAuGAGcGAGcATT-branching unit- |
| branching unit | GGGAAAcucuGucGuGGGAcGGucAGAcuGuucAAccAcuccucuuc |
| with siRNA_1 | |
| Core strand + | 5′ c*mA*amAumUcmCamUcmGu*mG*a-branching unit- |
| branching unit | GGGAAAcucuGucGuGGGAcGGucAGAcuGuucAAccAcuccucuuc |
| with siRNA_1 | |
| Double siRNA | 5′ Amine |
| with linker | c*mA*amAumUcmCamUcmGu*mG*aTTTTc*mA*amAumUcmCamUcmGu*mG*a |
| Branched | 5′ Cy3-c*mA*amAumUcmCamUcmGu*mG*a 3′-branching-3′ |
| siRNA | a*mG*umGcmUamCcmUumAa*mA*c-Cy3 5′ |

Stoichiometric amounts of the different strands (5 to 7) were combined in the assembly buffer (PBS+MgCl2 (2 mM)), with a final construct concentration of 10 μM. The strands were annealed to each other at 95° C. for 5 min then slowly cooled down to 4° C. (2.5° C./min), using a PCR thermocycler. The scaffold was then analyzed by native polyacrylamide gel electrophoresis (PAGE). For PAGE, the assembled scaffold was electrophoresed on native PAGE (6%) in 1× TBMg (890 mM Tris Borate+20 mM Mg(OAc)2, pH=8.3) at a constant voltage of 100 V. Gel bands were visualized using Cy3, then stained with GelRed™. 10 pmol of structures was loaded. 2 μL of glycerin (70% in H2O) was added to samples before loading (FIG. 37).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A composition comprising:
a nucleic acid nanoparticle, and
an oligomeric structure covalently linked to the nucleic acid nanoparticle.

2. The composition of claim 1, wherein the oligomeric structure is functionalized with a reactive group.

3. The composition of claim 2, wherein the oligomeric structure is covalently linked to a cargo molecule.

4. The composition of claim 3, wherein the covalently linked cargo molecule changes a physicochemical characteristic of the nucleic acid nanoparticle.

5. The composition of claim 3, wherein the cargo molecule is selected from the group consisting of a mRNA, gRNA/CRISPR, siRNA, ASO, miRNA, lnRNA, shRNA, ribozyme, aptamer, peptide, protein, antibody, therapeutic small molecule, lipid, cholesterol, synthetic polymer, amino acid, amino acid analogue, PEGS and hydrocarbon chain.

6. The composition of claim 2, wherein the oligomeric structure is linked to an RNA.

7. The composition of claim 6, wherein at least one of the nucleic acid nanoparticle and the RNA comprises a moiety that is reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

8. The composition of claim 2, wherein the reactive group comprises a phosphoramidite of formula (I): 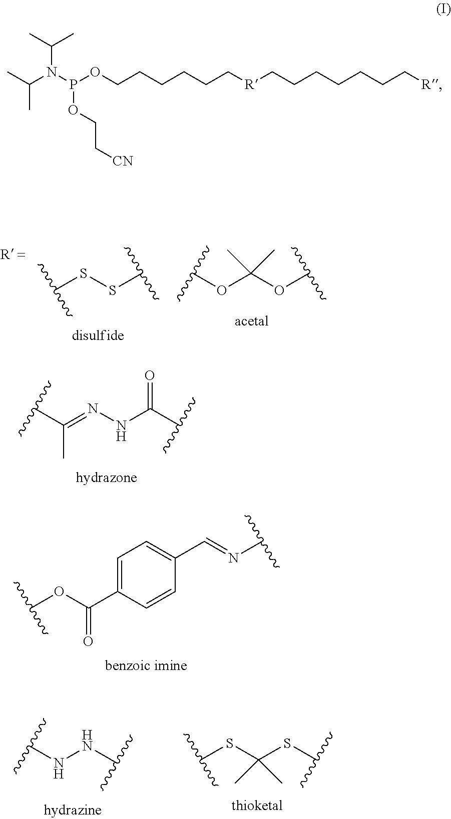
wherein:
R′ is a selected from the group consisting of a redox-responsive disulfide pH responsive hydrazone, hydrazine acetal, benzoic imine, and ROS-reactive thioketal, and
R″ is a reactive moiety that permits covalent conjugation.

9. The composition of claim 8, wherein R″ is reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

10. The composition of claim 8, wherein R″ is synthesized from a precursor selected from the group consisting of ADIBO-PEG4, N-[(1R,8S,9s)-bicyclo[6.1.0]non-4-yn-9-ylmethyloxycarbonyl]-1,8-diamino-3,6-dioxaoctane, (1R,8S,9s)-bicyclo[6.1.0]non-4-yn-9-ylmethanol, bromoacetamido-dPEG®4-amido-DBCO, bromoacetamido-dPEG®12-amido-DBCO, bromoacetamido-dPEG®24-amido-DBCO, dibenzocyclooctyne-acid, dibenzocyclooctyne-N-hydroxysuccinimidyl ester, dibenzocyclooctyne-PEG4-acid, dibenzocyclooctyne-PEG4-alcohol, dibenzocyclooctyne-PEG4-N-hydroxysuccinimidyl ester, (4-(1,2,4,5-tetrazin-3-yl)phenyl)methanamine hydrochloride, (E)-cyclooct-4-enol, (E)-cyclooct-4-enyl 2,5-dioxo-1-pyrrolidinyl carbonate, 2,5-Dioxo-1-pyrrolidinyl 5-[4-(1,2,4,5-tetrazin-3-yl)benzylamino]-5-oxopentanoate, 5-[4-(1,2,4,5-tetrazin-3-yl)benzylamino]-5-oxopentanoic acid, 5-norbornene-2-acetic acid succinimidyl ester, 5-norbornene-2-endo-acetic acid, methyltetrazine-NHS ester, methyltetrazine-PEG4-NHS ester, TCO PEG4 succinimidyl ester, TCO-amine, tetrazine-PEG5-NHS ester, alkyne-PEG5-acid, (R)-3-amino-5-hexynoic acid hydrochloride, (S)-3-amino-5-hexynoic acid hydrochloride, (S)-3-(boc-amino)-5-hexynoic acid, N-boc-4-pentyne-1-amine, boc-propargyl-Gly-OH, 3-ethynylaniline, 4-ethynylaniline, propargylamine hydrochloride, propargyl chloroformate, propargyl-N-hydroxysuccinimidyl ester, propargyl-PEG2-acid, 3-(4-azidophenyl)propionic acid, 3-azido-1-propanamine, 3-azido-1-propanol, 4-carboxybenzenesulfonazide, O-(2-aminoethyl)-O′-(2-azidoethyl)heptaethylene glycol, O-(2-aminoethyl)-O′-(2-azidoethyl)nonaethylene glycol, O-(2-aminoethyl)-O′-(2-azidoethyl)pentaethylene glycol, azido-dPEG®4(m)acid, azido-dPEG® (n)-amine, azido-dPEG®4(o) NHS ester, azido-dPEG® (p)-TFP ester, 2-[2-(2-azidoethoxy)ethoxy]ethanol, O-(2-azidoethyl)-O-[2-(diglycolyl-amino)ethyl]heptaethylene glycol, O-(2-azidoethyl)heptaethylene glycol, O-(2-azidoethyl)-O′-methyl-triethylene glycol, O-(2-azidoethyl)-O′-methyl-undecaethylene glycol, 17-azido-3,6,9,12,15-pentaoxaheptadecan-1-amine, 14-azido-3,6,9,12-tetraoxatetradecanoic acid, 11-azido-3,6,9-trioxaundecan-1-amine, and bromoacetamido-dPEG® (q)azide,
wherein:
m is 4, 8, 12, or 24;
n is 7, 11, 23, or 35;
o is 4, 8, 12, or 24;
p is 4, 8, 12, 24, 36; and
q is 3, 11, or 23.

11. The composition of claim 2, wherein the reactive group comprises a phosphoramidite of formula (II): 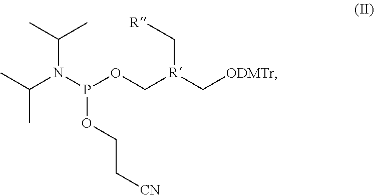
wherein:
R′ is CH or N; and
R″ is a reactive moiety that permits covalent conjugation.

12. The composition of claim 11, wherein R″ is reactive in a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

13. The composition of claim 11, wherein R″ is synthesized from a precursor selected from the group consisting of ADIBO-PEG4, N-[(1R,8S,9s)-bicyclo[6.1.0]non-4-yn-9-ylmethyloxycarbonyl]-1,8-diamino-3,6-dioxaoctane, (1R,8S,9s)-bicyclo[6.1.0]non-4-yn-9-ylmethanol, bromoacetamido-dPEG®4-amido-DBCO, bromoacetamido-dPEG® 12-amido-DBCO, bromoacetamido-dPEG®24-amido-DBCO, dibenzocyclooctyne-acid, dibenzocyclooctyne-N-hydroxysuccinimidyl ester, dibenzocyclooctyne-PEG4-acid, dibenzocyclooctyne-PEG4-alcohol, dibenzocyclooctyne-PEG4-N-hydroxysuccinimidyl ester, (4-(1,2,4,5-tetrazin-3-yl)phenyl)methanamine hydrochloride, (E)-cyclooct-4-enol, (E)-cyclooct-4-enyl 2,5-dioxo-1-pyrrolidinyl carbonate, 2,5-Dioxo-1-pyrrolidinyl 5-[4-(1,2,4,5-tetrazin-3-yl)benzylamino]-5-oxopentanoate, 5-[4-(1,2,4,5-tetrazin-3-yl)benzylamino]-5-oxopentanoic acid, 5-norbornene-2-acetic acid succinimidyl ester, 5-norbornene-2-endo-acetic acid, methyltetrazine-NHS ester, methyltetrazine-PEG4-NHS ester, TCO PEG4 succinimidyl ester, TCO-amine, tetrazine-PEG5-NHS ester, alkyne-PEG5-acid, (R)-3-amino-5-hexynoic acid hydrochloride, (S)-3-amino-5-hexynoic acid hydrochloride, (S)-3-(boc-amino)-5-hexynoic acid, N-boc-4-pentyne-1-amine, boc-propargyl-Gly-OH, 3-ethynylaniline, 4-ethynylaniline, propargylamine hydrochloride, propargyl chloroformate, propargyl-N-hydroxysuccinimidyl ester, propargyl-PEG2-acid, 3-(4-azidophenyl)propionic acid, 3-azido-1-propanamine, 3-azido-1-propanol, 4-carboxybenzenesulfonazide, O-(2-aminoethyl)-O′-(2-azidoethyl)heptaethylene glycol, O-(2-aminoethyl)-O′-(2-azidoethyl)nonaethylene glycol, O-(2-aminoethyl)-O′-(2-azidoethyl)pentaethylene glycol, azido-dPEG®4(m)acid, azido-dPEG® (n)-amine, azido-dPEG®4(o) NHS ester, azido-dPEG® (p)-TFP ester, 2-[2-(2-azidoethoxy)ethoxy]ethanol, O-(2-azidoethyl)-O-[2-(diglycolyl-amino)ethyl]heptaethylene glycol, O-(2-azidoethyl)heptaethylene glycol, O-(2-azidoethyl)-O′-methyl-triethylene glycol, O-(2-azidoethyl)-O′-methyl-undecaethylene glycol, 17-azido-3,6,9,12,15-pentaoxaheptadecan-1-amine, 14-azido-3,6,9,12-tetraoxatetradecanoic acid, 11-azido-3,6,9-trioxaundecan-1-amine, and bromoacetamido-dPEG® (q)azide,
wherein:
m is 4, 8, 12, or 24;
n is 7, 11, 23, or 35;
is 4, 8, 12, or 24;
p is 4, 8, 12, 24, 36; and
q is 3, 11, or 23.

14. The composition of claim 1, wherein the nucleic acid nanoparticle comprises a nucleic acid component comprising a base selected from the group consisting of 2′-deoxyinosine, 2′-deoxynebularine. 3-nitropyrrole 2′-deoxynucleoside, 5′-nitroindole 2′-deoxynucleoside, 6H, 8H-3,4-dihydro-pyrimido[4,5-c][1,2] oxazin-7-one (P), and 2-amino-9-(2-deoxy-β-ribofuranosyl)-6-methoxyaminopurine.

15. The composition of claim 14, wherein the nucleic acid component comprises a reactive group.

16. The composition of claim 15, wherein the reactive group comprises a phosphoramidite of formula (III): 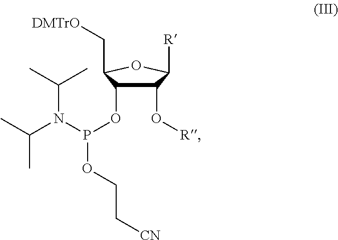
wherein:
R′ is selected from the group consisting of 2′-deoxyinosine, 2′-deoxynebularine. 3-nitropyrrole 2′-deoxynucleoside, 5′-nitroindole 2′-deoxynucleoside, 6H, 8H-3,4-dihydro-pyrimido[4,5-c][1,2] oxazin-7-one (P), and 2-amino-9-(2-deoxy-β-ribofuranosyl)-6-methoxyaminopurine, adenine, guanine, cytosine, thymine, and uridine; and
R″ is reactive in a reaction a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation.

17. The composition of claim 1, wherein the nucleic acid nanoparticle comprises a first conditionally-cleavable linker comprising a phosphoramidite.

18. The composition of claim 17, wherein the nucleic acid nanoparticle comprises a second conditionally-cleavable linker comprising a phosphoramidite, the second conditionally-cleavable linker being different from the first conditionally-cleavable linker.

19. The composition of claim 18, wherein:
cleavability of the first conditionally-cleavable linker is pH-sensitive; and
cleavability of the second conditionally-cleavable linker is redox-sensitive.

20. The composition of claim 1, further comprising:
a targeting moiety linked to the nanoparticle.

21. The composition of claim 20, wherein the targeting moiety is selected from the group consisting of a small molecule, a peptide, and an aptamer.

22. The composition of claim 1, further comprising:
a therapeutic moiety linked to the nanoparticle.

23. The composition of claim 22, wherein the therapeutic moiety is selected from the group consisting of a small molecule and a nucleic acid.

24. The composition of claim 22, further comprising:
a targeting moiety linked to the nanoparticle.

25. The composition of claim 1, wherein the nucleic acid nanoparticle comprises a nucleic acid that comprises a phosphoramidite of formula (IV):

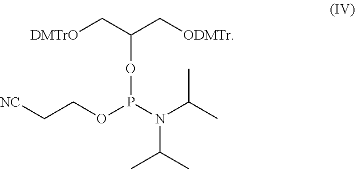

26. The composition of claim 1, wherein the nucleic acid nanoparticle comprises a nucleic acid that comprises a phosphoramidite of formula (V):

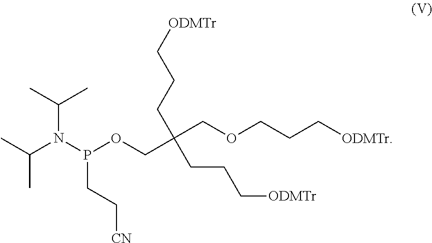

27. The composition of claim 1, wherein the nucleic acid nanoparticle comprises a nucleic acid that comprises a phosphoramidite of formula (VI):

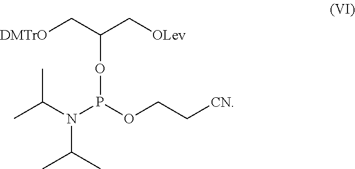

28. The composition of claim 1, wherein the nucleic acid nanoparticle comprises a nucleic acid that comprises a phosphoramidite of formula (VI):

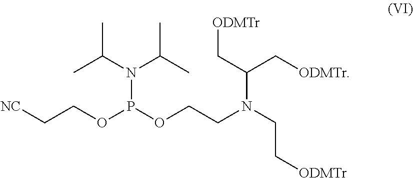

29. A method comprising attaching a nucleic acid nanoparticle to at least one RNA molecule via a reaction selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reaction (spring-loaded reactions), and traceless Staudinger ligation, wherein the reaction comprises at least one of the following conditions:
the reaction is carried out on the nucleic acid nanoparticle;
the reaction generates minimal by-products and comprises a high thermodynamic driving force that affords a single reaction product; and
the reaction is orthogonal to other reactive moieties present on the nucleic acid nanoparticle.

30. The method of claim 29, wherein the at least one RNA molecule is attached to a nucleic acid within the nanoparticle.

31. The method of claim 30, wherein the method comprises:
attaching a first cargo molecule to a first reactive moiety on the nucleic acid; and
attaching a second cargo molecule to a second reactive moiety on the nucleic acid, wherein the attaching steps are performed in the same reaction vessel.

32. The method of claim 31, wherein the cargo molecule is RNA.

33. A composition comprising:
a nucleic acid nanoparticle; and
a peptide covalently linked to the nucleic acid nanoparticle.

34. The composition of claim 33, wherein the nucleic acid nanoparticle is covalently linked to an oligomeric structure.

35. The composition of claim 34, wherein at least one of the peptide and the oligomeric structure comprises a reactive group.

36. The composition of claim 33, wherein the peptide is covalently linked to a 3′ end of a nucleic acid in the nanoparticle.

37. The composition of claim 33, wherein the peptide is covalently linked to a 5′ end of a nucleic acid in the nanoparticle.

38. The composition of claim 33, wherein the peptide is covalently linked to an internal portion of a nucleic acid in the nanoparticle.

39. The composition of claim 33, wherein the peptide comprises a sequence selected from the group consisting of GFWFG, GLFGAIAGFIENGWEGMIDGWYG, GLFEAIEGFIENGWEGMIDGWYG, LAEALAEALEALAA, WEAKLAKALAKALAKHLAKALAKALKACEA, Poly(Arg), Poly(Glu), Poly(His), and Poly(Leu).

40. The composition of claim 39, wherein the composition comprises a plurality of copies of the peptide, and wherein each of the plurality of copies is covalently linked to the nucleic acid nanoparticle is covalently at a different site within the peptide.

41. A composition comprising:
a nucleic acid nanoparticle comprising a nucleic acid that comprises a L-RNA base; and
a cargo molecule covalently linked to the nucleic acid nanoparticle

42. The composition of claim 41, wherein a portion of the cargo molecule is hybridized to a portion of the nucleic acid nanoparticle.

43. The composition of claim 41, wherein the cargo molecule comprises a L-RNA base.

44. A method comprising:
conjugating a nucleic acid nanoparticle to a targeting moiety; and
conjugating the nucleic acid nanoparticle to a therapeutic moiety.

45. The method of claim 44, wherein the conjugating step comprises a chemical reaction selected from the group consisting of CuAAC, RuAAC, IEDDA, SPAAC, NHS chemistry, thiol-maleimide, disulfide formation, and oxime formation.

46. The method of claim 45, further comprising conjugating a component to a nucleic acid in one of the nucleic acid nanoparticles, the targeting moiety, and the therapeutic moiety, wherein the nucleic acid comprises a modified nucleotide, and wherein the component is conjugated to a 3′ end or a 5′ end of the nucleic acid.

47. The method of claim 46, the conjugating steps are performed in a sequence that is determined by stability of covalent bonds produced in each conjugating step.

48. A composition comprising a nucleic acid nanostructure comprising a nucleic acid component comprising:
a single-stranded nucleic acid scaffold comprising DNA, RNA, or xeno-nucleic acid (XNA); and
a single-stranded staple comprising DNA, RNA, or xeno-nucleic acid, the single-stranded staple being bound to the scaffold via a scaffold-binding sequence in the staple that is complementary to a staple-binding sequence in the scaffold.

49. The composition of claim 48, wherein the scaffold is a nucleic acid therapeutic.

50. The composition of claim 49, wherein the nucleic acid therapeutic is an mRNA molecule.

51. The composition of claim 50, wherein the scaffold is an in vitro transcription (IVT) mRNA molecule.

52. The composition of claim 50, wherein the scaffold is selected from the group consisting of unmodified mRNA, nucleoside-modified mRNA, self-amplifying mRNA, and trans-amplifying mRNA.

53. The composition of claim 50, wherein the mRNA molecule has a length of at least 300 nucleotides.

54. The composition of claim 50, wherein the mRNA molecule is capped with a cap0 modification at its 5′-end.

55. The composition of claim 50, wherein the mRNA molecule is capped with a cap1 modification at its 5′-end.

56. The composition of claim 50, wherein the mRNA molecule is capped with a cap2 modification at its 5′-end.

57. The composition of claim 50, wherein the mRNA molecule has a poly(A) tail with a length of at least 120 adenosines at its 3′-end.

58. The composition of claim 50, wherein the mRNA molecule comprises an IRES sequence of encephalomyocarditis virus upstream of the open reading frame.

59. The composition of claim 50, wherein at least one uridine in the mRNA molecule is substituted by 5-methoxyuridine.

60. The composition of claim 50, wherein at least one uridine in the mRNA molecule is substituted by pseudouridine.

61. The composition of claim 50, wherein at least one cytidine in the mRNA molecule is substituted by 5-methylcytosine.

62. The composition of claim 50, wherein the staple strand comprises a plurality of non-overlapping scaffold-binding sequences.

63. The composition of claim 50, wherein the mRNA molecule comprises a plurality of non-overlapping staple-binding sequences

64. The composition of claim 63, wherein the plurality of non-overlapping staple-binding sequences in the mRNA molecule are absent from 5′ non-coding region and the 3′ UTR of the mRNA molecule.

65. The composition of claim 50, wherein mRNA molecule comprises a bundle comprising multiple helixes that are connected by a strand crossover.

66. The composition of claim 65, wherein the mRNA molecule comprises multiple bundles.

67. The composition of claim 65, wherein the mRNA molecule comprises a tube that comprises at least six bundles.

68. The composition of claim 50, wherein the mRNA molecule comprises a ‘zigzag’ scaffold routing pattern.

69. The composition of claim 50, wherein the mRNA comprises a ‘seam’ scaffold routing pattern.

70. The composition of claim 48, wherein complementarity between the staple-binding sequence and the scaffold-binding sequence is perfect complementarity.

71. The composition of claim 48, wherein the staple comprises DNA.

72. The composition of claim 48, wherein the staple has a length of at least 5 nucleotides.

73. The composition of claim 48, wherein the composition comprises a plurality of single-stranded staples.

74. The composition of claim 73, wherein the plurality of single-stranded staples comprises at least two of the following:
a staple comprising DNA;
a staple comprising RNA; and
a staple comprising XNA.

75. The composition of claim 73, wherein each of the plurality of single-stranded staples has a length of 20-60 nucleotides.

76. The composition of claim 73, wherein the plurality of single-stranded staples comprises:
at least one staple having a length of 10-35 nucleotides; and
at least one staple having a length of 36-80 nucleotides.

77. The composition of claim 48, wherein binding between the scaffold and the staple comprises at least one of the following:
uracil-containing DNA in at least one of the scaffold-binding sequence and the staple-binding sequence;
a mismatched base pair between the and the scaffold-binding sequence and staple-binding sequence;
a 5′ overhang adjacent at least one of the scaffold-binding sequence and the staple-binding sequence;
a 3′ overhang adjacent at least one of the scaffold-binding sequence and the staple-binding sequence;
<50% G+C content in at least one of the scaffold-binding sequence and the staple-binding sequence;
a region of complementarity between the scaffold-binding sequence and the staple-binding sequence that is less than 20 nucleotides in length;
a restriction endonuclease site in the region of complementarity between the scaffold-binding sequence and the staple-binding sequence;
a portion adjacent an end of the region of complementarity that is rich in AT or AU base pairs;
a reducible disulfide linkage in the region of complementarity;
a pH-sensitive linkage in the region of complementarity;
a photocleavable linkage in the region of complementarity;

78. The composition of claim 48, wherein the composition comprises a plurality of nucleic acid staples bound to the staple.

79. A composition comprising a 2- or 3-dimensional nucleic acid nanostructure comprising one or more nucleic acid components, wherein the nucleic acid component comprises;
i. a single-stranded nucleic acid scaffold composed of an mRNA; and
ii. one or more single-stranded staples comprised of a DNA, or RNA, or XNA strand, which is at least partially complementary to the scaffold and binds to one or more complementary sequences on the scaffold; and
iii. the nucleic acid component promotes the biological function of the nucleic acid nanostructure. For example, for an mRNA nanostructure, the nucleic acid component promotes translation.

80. The composition of claim 79, wherein one or more DNA, or RNA, or XNA staple strands are elongated at the 3′- and/or 5′-end with a nucleic acid sequence that does not hybridize to the mRNA scaffold.

81. The composition of claim 80, wherein one or more DNA, or RNA, or XNA staple strands are elongated at the 3′- and/or 5′-end with functional sticky ends or toeholds.

82. The composition of claim 80, wherein one or more DNA, or RNA, or XNA staple strands are elongated at the 3′- and/or 5′-end with a non-functional space holder sequence, for example 5′-AAAAAA-3′.

83. The composition of claim 80, wherein modifications to staple free end are designed to alter the physicochemical properties.

84. The composition of claim 83, wherein one or more DNA, or RNA, or XNA staple strands contain modified nucleosides such as 2′F, 2′OMe or phosphorothioate linkages to increase resistance against nucleases.

85. The composition of claim 83, wherein one or more DNA, or RNA, or XNA staple strands contain unnatural nucleosides, for example but limited to d5SICS (6-methylisoquinoline-1-thione-2-yl) and dNaM (3-methoxy-2-naphthyl) for increased hydrophobicity.

86. The composition of claim 83, where in one or more DNA, or RNA, or XNA staples strands contain unnatural nucleosides that are 2′ modified with modifications that can alter biodistribution, for example, but not limited to guanine, histidine, PEGₙ (where n is the number of repeating ethylene glycol units, which can be any given number between 2-20), alkyl chains (Cₙ—where n is the number of carbon atoms in the alkyl chain, which can be any given number between 2-20).

87. The composition of claim 80, wherein the nucleic acid linker can act as a recruitment platform for nucleic acid-binding proteins or enzymes. Example linker elements are from the group consisting of, but not limited to AU-rich sequence elements, CU-rich sequence elements, polyA motif, IRES, nuclear localization signal.

88. The composition of claim 80, wherein the nucleic acid linker comprises a self-dimerization domain, for example and not limited to, a pRNA loop capable of forming loop-loop interactions.

89. The composition of claim 80, wherein the nucleic acid linker comprises an internal photocleavable modification, allowing for light-mediated release of the cargo molecule.

90. The composition of claim 85, wherein 1 or more DNA, or RNA, or XNA staple strands are covalently attached to a second nucleic acid strand through click chemistry at their 3′-end or 5′-end.

91. The composition of claim 79, wherein 1 or more DNA, or RNA, or XNA staple strands are directly functionalized with click chemistry handles from the group consisting of, but not limited to, acrydite, alkene, alkyne, amine, azide, cycloalkyne, epoxide, fluorosulfate, hydrazine, isocyanate, maleimide, nitrone, olefin, phosphine, tetrazine, thiol at their 3′-end, 5′-end or internally.

92. The composition of claim 79, wherein 1 or more DNA, or RNA, or XNA staple strands are enzymatically functionalized with a click handle (see Table 3) at their 3′-end, 5′-end or internally.

93. The composition of claim 79, wherein the mRNA scaffold strand is enzymatically functionalized with a click handle at its 3′-end, 5′-end or internally.

94. The composition of claim 79, wherein 1 or more DNA, or RNA, or XNA staple strands are elongated at their 3′ and/or 5′ end with an ON recognition sequence for HUH endonuclease-mediated protein conjugation (see Table 3).

95. The composition of claim 79, wherein 1 or more DNA, or RNA, or XNA staple strands are functionalized at their 3′ and/or 5′ end with a reducible, disulphide-containing crosslinker from the group consisting of, but not limited to, SPP, SPDB, sulfo-SPDB, SPDP (see Table 4).

96. The composition of claim 79, wherein 1 or more DNA, or RNA, or XNA staple strands are functionalized at their 3′ and/or 5′ end with a pH sensitive, acid cleavable linker from the group consisting of, but not limited to, acetyl butyrate, hydrazone, cis-aconityl, acetal (see Table 1C).

97. The composition of claim 79, wherein 1 or more DNA, or RNA, or XNA staple strands are functionalized at their 3′ and/or 5′ end with a protease sensitive di- or tripeptide linker from the group consisting of, but not limited to, Phe-Lys, Val-Ala, Val-Ci, Glu-Val-Cit, Phe-Lys-PABC, Val-Ci-PABC, and cBu-Cit-PABC (see Table 4).

98. The composition of claim 79, wherein 1 or more DNA, or RNA, or XNA staple strands are functionalized at their 3′ and/or 5′ end with a galactosidase- or glucuronic acid sensitive linker from the group consisting of, but not limited to, β-glucuronic acid-PABC, methylene-alkoxy-β-glucuronic acid-PABC, b-galactoside (see Table 4).

99. The composition of claim 79, wherein 1 or more DNA, or RNA, or XNA staple strands are functionalized at their 3′ and/or 5′ end with a non-cleavable linker from the group consisting of, but not limited to, SMCC, maleimidocaproyl linker, PEG4Mal, SMPB, SIAB (see Table 4).

100. The composition of claim 79, wherein 1 or more DNA, or RNA, or XNA staple strands are attached to n-alkyl linker at their 3′-end/or 5′-end carrying functional azide, alkyne, amine or thiol groups.

101. The composition of claim 79, wherein 1 or more DNA, or RNA, or XNA staple strands are attached to polyethylene glycol linker at their 3′-end/or 5′-end carrying functional click chemistry handles from the group consisting of, but not limited to, acrydite, alkene, alkyne, amine, azide, cycloalkyne, epoxide, fluorosulfate, hydrazine, isocyanate, maleimide, nitrone, olefin, phosphine, tetrazine, thiol.

102. A composition comprising:
A 2- or 3-dimensional nucleic acid nanostructure comprising one or more nucleic acid components, wherein the nucleic acid component comprises;
(i) a single-stranded nucleic acid scaffold composed of an mRNA; and
(ii) one or more single-stranded staples comprised of a DNA strand, which is at least partially complementary to the scaffold and binds to one or more complementary sequences on the scaffold

103. The composition of claim 103, wherein the mRNA is modified with one or more unnatural nucleosides that are 2′ modified with modifications that can alter biodistribution, for example, but not limited to guanine, histidine, PEGₙ (where n is the number of repeating ethylene glycol units, which can be any given number between 2-20), alkyl chains (Cₙ—where n is the number of carbon atoms in the alkyl chain, which can be any given number between 2-20).

104. The composition of claim 102, wherein one or more DNA, or RNA, or XNA staple or scaffold strands are conjugated to one or more cargo molecules. For example, and without limitation, the cargo may include one or more of the molecules shown in Table 5.

105. The composition of claim 104, wherein the cargo molecule comprises RNA or DNA from the group of, but not limited to, mRNA, microRNA, siRNA, shRNA, saRNA, lnRNA, antimir, ASO, gapmer, splice-switching oligomer, aptamer, spiegelmer, gRNA (CRIPSR, ADAR), plasmid, ribozyme, oligonucleotide barcode.

106. The composition of claim 105, wherein one or multiple nucleic acid cargoes are attached to one or multiple nucleic acid staple strands during solid-phase synthesis. For example, and without limitation, the cargo may be attached via a phosphodiester linkage, a phosphorothioate linkage or a reducible disulphide linkage.

107. The composition of claim 105, wherein one or multiple nucleic acid cargoes are attached to one or multiple nucleic acid staple strands via enzymatic ligation (Table 3).

108. The composition of claim 105, wherein one or multiple nucleic acid cargoes are attached to one or multiple nucleic acid staple strands via reactions selected from the group consisting of CuAAC, SPAAC, RuAAC, IEDDA, SuFEx, SPANC, hydrazone/oxime ether formation, thiol-ene radical reaction, thiol-yne radical reaction, thiol-Michael addition reaction, thiol-isocyanate reaction, thiol-epoxide click reaction, nucleophilic ring opening reactions (spring-loaded reactions), traceless Staudinger ligation.

109. The composition of claim 105, wherein one or multiple nucleic acid cargoes are non-covalently annealed to one or multiple nucleic acid staple strands through base-pairing interactions on a hybridization arm (sticky-bridge-type annealing). In some embodiments, the cargo can be linked to staple strands via toeholds whose sequence is complementary to both the cargo and the staple's sequence.

110. The composition of claim 102, wherein two or more scaffold strands are connected.

111. The composition of claim 110, wherein two or more scaffold strands are connected by one or more staple strands via complementary base pairing.

112. The composition of claim 110, wherein two or more scaffold strands are coupled via linkers. The attachments may be covalent or non-covalent. The attachments may be reversible or irreversible.


SEQUENCE LISTING (166 sequences)

SEQ ID NO: 1 - protein, 5 aa, artificial
  Cargo peptide
GFWFG

SEQ ID NO: 2 - protein, 23 aa, Artificial
  Cargo peptide
GLFGAIAGFIENGWEGMIDGWYG

SEQ ID NO: 3 - protein, 23 aa, Artificial Sequence
  Synthetic
GLFEAIEGFIENGWEGMIDGWYG

SEQ ID NO: 4 - protein, 14 aa, Artificial
  Cargo peptide
LAEALAEALEALAA

SEQ ID NO: 5 - protein, 30 aa, Artificial
  Cargo peptide
WEAKLAKALAKALAKHLAKALAKALKACEA

SEQ ID NO: 6 - RNA, 47 nt, artificial
  synthetic
  misc_feature (7)..(10): 2′ fluorine
  misc_feature (12)..(13): 2′ fluorine
  misc_feature (12)..(13): 2′ fluorine
  misc_feature (15)..(15): 2′ flourine
  misc_feature (20)..(20): 2′ fluorine
  misc_feature (23)..(24): 2′ fluorine
  misc_feature (28)..(29): 2′ fluorine
  misc_feature (31)..(33): 2′ fluorine
  misc_feature (36)..(37): 2′ fluorine
  misc_feature (39)..(47): 2′ fluorine
gggaaacucugucgugggacggucagacuguucaaccacuccucuuc

SEQ ID NO: 7 - DNA, 47 bp, Artificial
  Synthetic
  misc_feature (7)..(10): 2′ fluorine
  misc_feature (12)..(13): 2′ fluorine
  misc_feature (15)..(15): 2′ flourine
  misc_feature (20)..(20): 2′ fluorine
  misc_feature (23)..(24): 2′ fluorine
  misc_feature (28)..(29): 2′ fluorine
  misc_feature (31)..(33): 2′ fluorine
  misc_feature (36)..(37): 2′ fluorine
  misc_feature (39)..(47): 2′ fluorine
gggaaacucugucgugggacggucagacuguucaaccacuccucuuc

SEQ ID NO: 8 - DNA, 47 bp, Artificial Sequence; identical to SEQ ID NO: 7
  synthetic
  misc_feature (7)..(10): 2′ fluorine
  misc_feature (12)..(13): 2′ fluorine
  misc_feature (12)..(13): 2′ fluorine
  misc_feature (15)..(15): 2′ flourine
  misc_feature (20)..(20): 2′ fluorine
  misc_feature (23)..(24): 2′ fluorine
  misc_feature (28)..(29): 2′ fluorine
  misc_feature (31)..(33): 2′ fluorine
  misc_feature (36)..(37): 2′ fluorine
  misc_feature (39)..(47): 2′ fluorine

SEQ ID NO: 9 - DNA, 47 bp, Artificial Sequence; identical to SEQ ID NO: 7
  Synthetic
  misc_feature (1)..(6): 2′OMe
  misc_feature (7)..(10): 2′ fluorine
  misc_feature (10)..(10): 2′OMe
  misc_feature (11)..(11): 2′OMe
  misc_feature (12)..(13): 2′ fluorine
  misc_feature (14)..(14): 2′OMe
  misc_feature (15)..(15): 2′ flourine
  misc_feature (16)..(19): 2′OMe
  misc_feature (20)..(20): 2′ fluorine
  misc_feature (21)..(22): 2′OMe
  misc_feature (23)..(24): 2′ fluorine
  misc_feature (25)..(27): 2′OMe
  misc_feature (28)..(29): 2′ fluorine
  misc_feature (31)..(33): 2′ fluorine
  misc_feature (34)..(35): 2′OMe
  misc_feature (36)..(37): 2′ fluorine
  misc_feature (38)..(38): 2′OMe
  misc_feature (39)..(47): 2′ fluorine
  misc_feature (47)..(47): 5′ norbornene

SEQ ID NO: 10 - DNA, 47 bp, Artificial Sequence; identical to SEQ ID NO: 7
  Synthetic
  misc_feature (1)..(47): 2′OMe
  misc_feature (47)..(47): 5′ norbornene

SEQ ID NO: 11 - DNA, 47 bp, Artificial Sequence; identical to SEQ ID NO: 7
  Synthetic
  misc_feature (7)..(10): 2′ fluorine
  misc_feature (12)..(13): 2′ fluorine
  misc_feature (15)..(15): 2′ flourine
  misc_feature (20)..(20): 2′ fluorine
  misc_feature (23)..(24): 2′ fluorine
  misc_feature (28)..(29): 2′ fluorine
  misc_feature (31)..(33): 2′ fluorine
  misc_feature (36)..(37): 2′ fluorine
  misc_feature (39)..(47): 2′ fluorine
  misc_feature (47)..(47): conjugated to PPIB x2

SEQ ID NO: 12 - DNA, 47 bp, Artificial Sequence; identical to SEQ ID NO: 7
  Synthetic
  misc_feature (7)..(10)
  misc_feature (12)..(13)
  misc_feature (15)..(15): 2′ flourine
  misc_feature (20)..(20): 2′ fluorine
  misc_feature (23)..(24): 2′ fluorine
  misc_feature (28)..(29): 2′ fluorine
  misc_feature (31)..(33): 2′ fluorine
  misc_feature (36)..(37): 2′ fluorine
  misc_feature (39)..(47): 2′ fluorine
  misc_feature (47)..(47): Conjugated to PLK1

SEQ ID NO: 13 - DNA, 47 bp, Artificial Sequence
  Synthetic
  misc_feature (16)..(16): 2′ Fluorine
  misc_feature (20)..(20): 2′ Fluorine
  misc_feature (23)..(23): 2′ Fluorine
  misc_feature (25)..(26): 2′ Fluorine
  misc_feature (28)..(28): 2′ Fluorine
  misc_feature (29)..(29): 2′ Fluorine
  misc_feature (31)..(33): 2′ Fluorine
  misc_feature (35)..(37): 2′ Fluorine
  misc_feature (39)..(44): 2′ Fluorine
  misc_feature (47)..(47): 2′ Fluorine
gggaaagaagaggaguggacgguacuguguuucaaccugucucugac

SEQ ID NO: 14 - DNA, 47 bp, artificial; identical to SEQ ID NO: 13
  synthetic
  misc_feature (1)..(47): phosphorothioate backbone
  misc_feature (16)..(16): 2′ Fluorine
  misc_feature (20)..(20): 2′ Fluorine
  misc_feature (23)..(23): 2′ Fluorine
  misc_feature (25)..(26): 2′ Fluorine
  misc_feature (28)..(28): 2′ Fluorine
  misc_feature (29)..(29): 2′ Fluorine
  misc_feature (31)..(33): 2′ Fluorine
  misc_feature (35)..(37): 2′ Fluorine
  misc_feature (39)..(44): 2′ Fluorine
  misc_feature (47)..(47): 2′ Fluorine

SEQ ID NO: 15 - DNA, 47 bp, Artificial Sequence; identical to SEQ ID NO: 13
  Synthetic
  misc_feature (1)..(15): 2′Ome
  misc_feature (16)..(16): 2′ Fluorine
  misc_feature (17)..(19): 2′Ome
  misc_feature (20)..(20): 2′ Fluorine
  misc_feature (21)..(22): 2′Ome
  misc_feature (23)..(23): 2′ Fluorine
  misc_feature (24)..(24): 2′Ome
  misc_feature (25)..(26): 2′ Fluorine
  misc_feature (27)..(27): 2′Ome
  misc_feature (28)..(28): 2′ Fluorine
  misc_feature (29)..(29): 2′Ome
  misc_feature (29)..(29): 2′ Fluorine
  misc_feature (31)..(33): 2′ Fluorine
  misc_feature (33)..(34): 2′Ome
  misc_feature (35)..(36): 2′Ome
  misc_feature (35)..(37): 2′ Fluorine
  misc_feature (39)..(39): 2′Ome
  misc_feature (39)..(44): 2′ Fluorine
  misc_feature (47)..(47): 2′ Fluorine

SEQ ID NO: 16 - DNA, 47 bp, Artificial Sequence; identical to SEQ ID NO: 13
  Synthetic
  misc_feature (1)..(47): histamine azide clicked onto the 2′ position of A at 8 locations
  misc_feature (16)..(16): 2′ Fluorine
  misc_feature (20)..(20): 2′ Fluorine
  misc_feature (23)..(23): 2′ Fluorine
  misc_feature (25)..(26): 2′ Fluorine
  misc_feature (28)..(28): 2′ Fluorine
  misc_feature (29)..(29): 2′ Fluorine
  misc_feature (31)..(33): 2′ Fluorine
  misc_feature (35)..(37): 2′ Fluorine
  misc_feature (39)..(44): 2′ Fluorine
  misc_feature (47)..(47): 2′ Fluorine

SEQ ID NO: 17 - DNA, 47 bp, Artificial Sequence; identical to SEQ ID NO: 13
  Synthetic
  misc_feature (4)..(6): 1-azidoododecane clicked onto 2′ position
  misc_feature (8)..(9): 1-azidoododecane clicked onto 2′ position
  misc_feature (11)..(11): 1-azidoododecane clicked onto 2′ position
  misc_feature (14)..(14): 1-azidoododecane clicked onto 2′ position
  misc_feature (16)..(16): 2′ Fluorine
  misc_feature (19)..(19): 1-azidoododecane clicked onto 2′ position
  misc_feature (20)..(20): 2′ Fluorine
  misc_feature (23)..(23): 2′ Fluorine
  misc_feature (25)..(26): 2′ Fluorine
  misc_feature (28)..(28): 2′ Fluorine
  misc_feature (29)..(29): 2′ Fluorine
  misc_feature (31)..(33): 2′ Fluorine
  misc_feature (34)..(35): 1-azidododecane clicked onto 2′ position
  misc_feature (35)..(37): 2′ Fluorine
  misc_feature (39)..(44): 2′ Fluorine
  misc_feature (46)..(46): 1-azidododecane clicked onto 2′ position
  misc_feature (47)..(47): 2′ Fluorine

SEQ ID NO: 18 - DNA, 47 bp, artificial; identical to SEQ ID NO: 13
  synthetic
  misc_feature (4)..(6): arginine azide clicked at 2′
  misc_feature (8)..(9): arginine azide clicked at 2′
  misc_feature (11)..(11): arginine azide clicked at 2′
  misc_feature (14)..(14): arginine azide clicked at 2′
  misc_feature (16)..(16): 2′ Fluorine
  misc_feature (19)..(19): arginine azide clicked at 2′
  misc_feature (20)..(20): 2′ Fluorine
  misc_feature (23)..(23): 2′ Fluorine
  misc_feature (24)..(24): arginine azide clicked at 2′
  misc_feature (25)..(26): 2′ Fluorine
  misc_feature (28)..(28): 2′ Fluorine
  misc_feature (29)..(29): 2′ Fluorine
  misc_feature (31)..(33): 2′ Fluorine
  misc_feature (34)..(35): arginine azide clicked at 2′
  misc_feature (35)..(37): 2′ Fluorine
  misc_feature (39)..(44): 2′ Fluorine
  misc_feature (46)..(46): arginine azide clicked at 2′
  misc_feature (47)..(47): 2′ Fluorine

SEQ ID NO: 19 - DNA, 47 bp, artificial; identical to SEQ ID NO: 13
  Synthetic
  misc_feature (4)..(6): PEG-7 clicked on 2′
  misc_feature (8)..(9): PEG-7 clicked on 2′
  misc_feature (11)..(11): PEG-7 clicked on 2′
  misc_feature (14)..(14): PEG-7 clicked on 2′
  misc_feature (16)..(16): 2′ Fluorine
  misc_feature (19)..(19): PEG-7 clicked on 2′
  misc_feature (20)..(20): 2′ Fluorine
  misc_feature (23)..(23): 2′ Fluorine
  misc_feature (24)..(24): PEG-7 clicked on 2′
  misc_feature (25)..(26): 2′ Fluorine
  misc_feature (28)..(28): 2′ Fluorine
  misc_feature (29)..(29): 2′ Fluorine
  misc_feature (31)..(33): 2′ Fluorine
  misc_feature (34)..(35): PEG-7 clicked on 2′
  misc_feature (35)..(37): 2′ Fluorine
  misc_feature (39)..(44): 2′ Fluorine
  misc_feature (46)..(46): PEG-7 clicked on 2′
  misc_feature (47)..(47): 2′ Fluorine

SEQ ID NO: 20 - DNA, 47 bp, artificial; identical to SEQ ID NO: 13
  Synthetic
  misc_feature (16)..(16): 2′ Fluorine
  misc_feature (20)..(20): 2′ Fluorine
  misc_feature (23)..(23): 2′ Fluorine
  misc_feature (25)..(26): 2′ Fluorine
  misc_feature (28)..(28): 2′ Fluorine
  misc_feature (29)..(29): 2′ Fluorine
  misc_feature (31)..(33): 2′ Fluorine
  misc_feature (35)..(37): 2′ Fluorine
  misc_feature (39)..(44): 2′ Fluorine
  misc_feature (47)..(47): 5′ TEG cholesterol
  misc_feature (47)..(47): 2′ Fluorine

SEQ ID NO: 21 - DNA, 47 bp, artificial; identical to SEQ ID NO: 13
  Synthetic
  misc_feature (1)..(47): 2′Ome

SEQ ID NO: 22 - DNA, 47 bp, artificial; identical to SEQ ID NO: 13
  Synthetic
  misc_feature (16)..(16): 2′ Fluorine
  misc_feature (20)..(20): 2′ Fluorine
  misc_feature (23)..(23): 2′ Fluorine
  misc_feature (25)..(26): 2′ Fluorine
  misc_feature (28)..(28): 2′ Fluorine
  misc_feature (29)..(29): 2′ Fluorine
  misc_feature (31)..(33): 2′ Fluorine
  misc_feature (35)..(37): 2′ Fluorine
  misc_feature (39)..(44): 2′ Fluorine
  misc_feature (47)..(47): conjugated to linear peptide GFWFG
  misc_feature (47)..(47): 2′ Fluorine

SEQ ID NO: 23 - DNA, 47 bp, Artificial
  Synthetic
  misc_feature (8)..(8): 2′ Fluorine
  misc_feature (11)..(11): 2′ Fluorine
  misc_feature (13)..(13): 2′ Fluorine
  misc_feature (16)..(16): 2′ Fluorine
  misc_feature (20)..(20): 2′ Fluorine
  misc_feature (23)..(23): 2′ Fluorine
  misc_feature (25)..(25): 2′ Fluorine
  misc_feature (27)..(28): 2′ Fluorine
  misc_feature (31)..(33): 2′ Fluorine
  misc_feature (36)..(38): 2′ Fluorine
  misc_feature (40)..(40): 2′ Fluorine
  misc_feature (43)..(43): 2′ Fluorine
gggaaagcaguguagcggacggugugucaguucaacccacgacagag

SEQ ID NO: 24 - DNA, 47 bp, Artificial; identical to SEQ ID NO: 23
  Synthetic
  misc_feature (1)..(47): phosphorothioate backbone
  misc_feature (8)..(8): 2′ Fluorine
  misc_feature (11)..(11): 2′ Fluorine
  misc_feature (13)..(13): 2′ Fluorine
  misc_feature (16)..(16): 2′ Fluorine
  misc_feature (20)..(20): 2′ Fluorine
  misc_feature (23)..(23): 2′ Fluorine
  misc_feature (25)..(25): 2′ Fluorine
  misc_feature (27)..(28): 2′ Fluorine
  misc_feature (31)..(33): 2′ Fluorine
  misc_feature (36)..(38): 2′ Fluorine
  misc_feature (40)..(40): 2′ Fluorine
  misc_feature (43)..(43): 2′ Fluorine

SEQ ID NO: 25 - DNA, 47 bp, Artificial; identical to SEQ ID NO: 23
  Synthetic
  misc_feature (1)..(7): 2′Ome
  misc_feature (8)..(8): 2′ Fluorine
  misc_feature (9)..(10): 2′Ome
  misc_feature (11)..(11): 2′ Fluorine
  misc_feature (12)..(12): 2′Ome
  misc_feature (13)..(13): 2′ Fluorine
  misc_feature (14)..(15): 2′Ome
  misc_feature (16)..(16): 2′ Fluorine
  misc_feature (17)..(19): 2′Ome
  misc_feature (20)..(20): 2′ Fluorine
  misc_feature (21)..(22): 2′Ome
  misc_feature (23)..(23): 2′ Fluorine
  misc_feature (24)..(24): 2′Ome
  misc_feature (25)..(25): 2′ Fluorine
  misc_feature (26)..(26): 2′Ome
  misc_feature (27)..(28): 2′ Fluorine
  misc_feature (29)..(30): 2′Ome
  misc_feature (31)..(33): 2′ Fluorine
  misc_feature (34)..(35): 2′Ome
  misc_feature (36)..(38): 2′ Fluorine
  misc_feature (39)..(39): 2′Ome
  misc_feature (40)..(40): 2′ Fluorine
  misc_feature (42)..(42): 2′Ome
  misc_feature (43)..(43): 2′ Fluorine
  misc_feature (44)..(47): 2′Ome

SEQ ID NO: 26 - DNA, 47 bp, Artificial; identical to SEQ ID NO: 23
  Synthetic
  misc_feature (1)..(47): histamine azide clicked on 2′ of A at 8 locations
  misc_feature (8)..(8): 2′ Fluorine
  misc_feature (11)..(11): 2′ Fluorine
  misc_feature (13)..(13): 2′ Fluorine
  misc_feature (16)..(16): 2′ Fluorine
  misc_feature (20)..(20): 2′ Fluorine
  misc_feature (23)..(23): 2′ Fluorine
  misc_feature (25)..(25): 2′ Fluorine
  misc_feature (27)..(28): 2′ Fluorine
  misc_feature (31)..(33): 2′ Fluorine
  misc_feature (36)..(38): 2′ Fluorine
  misc_feature (40)..(40): 2′ Fluorine
  misc_feature (43)..(43): 2′ Fluorine

SEQ ID NO: 27 - DNA, 47 bp, Artificial; identical to SEQ ID NO: 23
  Synthetic
  misc_feature (4)..(6): PEG-7 clicked at 2′
  misc_feature (8)..(8): 2′ Fluorine
  misc_feature (9)..(9): PEG-7 clicked at 2′
  misc_feature (11)..(11): 2′ Fluorine
  misc_feature (13)..(13): 2′ Fluorine
  misc_feature (14)..(14): PEG-7 clicked at 2′
  misc_feature (16)..(16): 2′ Fluorine
  misc_feature (19)..(19): PEG-7 clicked at 2′
  misc_feature (20)..(20): 2′ Fluorine
  misc_feature (23)..(23): 2′ Fluorine
  misc_feature (25)..(25): 2′ Fluorine
  misc_feature (27)..(28): 2′ Fluorine
  misc_feature (29)..(29): PEG-7 clicked at 2′
  misc_feature (31)..(33): 2′ Fluorine
  misc_feature (34)..(35): PEG-7 clicked at 2′
  misc_feature (36)..(38): 2′ Fluorine
  misc_feature (39)..(39): PEG-7 clicked at 2′
  misc_feature (40)..(40): 2′ Fluorine
  misc_feature (41)..(41): PEG-7 clicked at 2′
  misc_feature (43)..(43): 2′ Fluorine
  misc_feature (44)..(44): PEG-7 clicked at 2′
  misc_feature (46)..(46): PEG-7 clicked at 2′
  misc_feature (46)..(46): PEG-7 clicked at 2′

SEQ ID NO: 28 - DNA, 47 bp, Artificial; identical to SEQ ID NO: 23
  Synthetic
  misc_feature (4)..(6): arginine azide clicked onto 2′ position
  misc_feature (8)..(8): 2′ Fluorine
  misc_feature (9)..(9): arginine azide clicked onto 2′ position
  misc_feature (11)..(11): 2′ Fluorine
  misc_feature (13)..(13): 2′ Fluorine
  misc_feature (14)..(14): arginine azide clicked onto 2′ position
  misc_feature (16)..(16): 2′ Fluorine
  misc_feature (19)..(19): arginine azide clicked onto 2′ position
  misc_feature (20)..(20): 2′ Fluorine
  misc_feature (23)..(23): 2′ Fluorine
  misc_feature (25)..(25): 2′ Fluorine
  misc_feature (27)..(28): 2′ Fluorine
  misc_feature (29)..(29): arginine azide clicked onto 2′ position
  misc_feature (31)..(33): 2′ Fluorine
  misc_feature (34)..(35): arginine azide clicked onto 2′ position
  misc_feature (36)..(38): 2′ Fluorine
  misc_feature (39)..(39): arginine azide clicked onto 2′ position
  misc_feature (40)..(40): 2′ Fluorine
  misc_feature (42)..(42): arginine azide clicked onto 2′ position
  misc_feature (43)..(43): 2′ Fluorine
  misc_feature (44)..(44): arginine azide clicked onto 2′ position
  misc_feature (46)..(46): arginine azide clicked onto 2′ position

SEQ ID NO: 29 - DNA, 47 bp, Artificial; identical to SEQ ID NO: 23
  Synthetic
  misc_feature (47)..(47): extended region for hybridization of aptamer sequence

SEQ ID NO: 30 - DNA, 47 bp, Artificial; identical to SEQ ID NO: 23
  Synthetic
  misc_feature (8)..(8): 2′ Fluorine
  misc_feature (11)..(11): 2′ Fluorine
  misc_feature (13)..(13): 2′ Fluorine
  misc_feature (16)..(16): 2′ Fluorine
  misc_feature (20)..(20): 2′ Fluorine
  misc_feature (23)..(23): 2′ Fluorine
  misc_feature (25)..(25): 2′ Fluorine
  misc_feature (27)..(28): 2′ Fluorine
  misc_feature (31)..(33): 2′ Fluorine
  misc_feature (36)..(38): 2′ Fluorine
  misc_feature (40)..(40): 2′ Fluorine
  misc_feature (43)..(43): 2′ Fluorine
  misc_feature (47)..(47): extends to region for hybridization of aptamer sequence

SEQ ID NO: 31 - DNA, 47 bp, Artificial
  Synthetic
  misc_feature (8)..(9): 2′ Fluorine
  misc_feature (15)..(15): 2′ Fluorine
  misc_feature (20)..(20): 2′ Fluorine
  misc_feature (23)..(25): 2′ Fluorine
  misc_feature (29)..(37): 2′ Fluorine
  misc_feature (39)..(40): 2′ Fluorine
  misc_feature (42)..(42): 2′ Fluorine
  misc_feature (44)..(45): 2′ Fluorine
  misc_feature (47)..(47): 2′ Fluorine
gggaaagucagagacaggacggucuaggucuucaaccgcuacacugc

SEQ ID NO: 32 - DNA, 47 bp, Artificial; identical to SEQ ID NO: 31
  Synthetic
  misc_feature (8)..(9): 2′ Fluorine
  misc_feature (15)..(15): 2′ Fluorine
  misc_feature (20)..(20): 2′ Fluorine
  misc_feature (23)..(25): 2′ Fluorine
  misc_feature (29)..(37): 2′ Fluorine
  misc_feature (39)..(40): 2′ Fluorine
  misc_feature (42)..(42): 2′ Fluorine
  misc_feature (44)..(45): 2′ Fluorine
  misc_feature (47)..(47): 5′ norbornene
  misc_feature (47)..(47): 2′ Fluorine

SEQ ID NO: 33 - DNA, 47 bp, Artificial; identical to SEQ ID NO: 31
  Synthetic
  misc_feature (1)..(47): phosphorothioate backbone
  misc_feature (8)..(9): 2′ Fluorine
  misc_feature (15)..(15): 2′ Fluorine
  misc_feature (20)..(20): 2′ Fluorine
  misc_feature (23)..(25): 2′ Fluorine
  misc_feature (29)..(37): 2′ Fluorine
  misc_feature (39)..(40): 2′ Fluorine
  misc_feature (42)..(42): 2′ Fluorine
  misc_feature (44)..(45): 2′ Fluorine
  misc_feature (47)..(47): 5′ norbornene
  misc_feature (47)..(47): 2′ Fluorine

SEQ ID NO: 34 - DNA, 47 bp, Artificial; identical to SEQ ID NO: 31
  Synthetic
  misc_feature (1)..(7): 2′Ome
  misc_feature (8)..(9): 2′ Fluorine
  misc_feature (10)..(14): 2′Ome
  misc_feature (15)..(15): 2′ Fluorine
  misc_feature (16)..(19): 2′Ome
  misc_feature (20)..(20): 2′ Fluorine
  misc_feature (21)..(22): 2′Ome
  misc_feature (23)..(25): 2′ Fluorine
  misc_feature (25)..(27): 2′Ome
  misc_feature (29)..(37): 2′ Fluorine
  misc_feature (34)..(35): 2′Ome
  misc_feature (37)..(37): 2′Ome
  misc_feature (39)..(40): 2′ Fluorine
  misc_feature (41)..(41): 2′Ome
  misc_feature (42)..(42): 2′ Fluorine
  misc_feature (43)..(43): 2′Ome
  misc_feature (44)..(45): 2′ Fluorine
  misc_feature (46)..(46): 2′Ome
  misc_feature (47)..(47): 2′ Fluorine

SEQ ID NO: 35 - DNA, 47 bp, Artificial; identical to SEQ ID NO: 31
  Synthetic
  misc_feature (1)..(47): 2′ Moe
  misc_feature (47)..(47): 5′ norbornene

SEQ ID NO: 36 - DNA, 47 bp, Artificial; identical to SEQ ID NO: 31
  Synthetic
  misc_feature (8)..(9): 2′ Fluorine
  misc_feature (15)..(15): 2′ Fluorine
  misc_feature (20)..(20): 2′ Fluorine
  misc_feature (23)..(25): 2′ Fluorine
  misc_feature (29)..(37): 2′ Fluorine
  misc_feature (39)..(40): 2′ Fluorine
  misc_feature (42)..(42): 2′ Fluorine
  misc_feature (44)..(45): 2′ Fluorine
  misc_feature (47)..(47): conjugated to 2x PPIB
  misc_feature (47)..(47): 2′ Fluorine

SEQ ID NO: 37 - DNA, 47 bp, Artificial; identical to SEQ ID NO: 31
  39
  misc_feature (8)..(9): 2′ Fluorine
  misc_feature (15)..(15): 2′ Fluorine
  misc_feature (20)..(20): 2′ Fluorine
  misc_feature (23)..(25): 2′ Fluorine
  misc_feature (29)..(37): 2′ Fluorine
  misc_feature (39)..(40): 2′ Fluorine
  misc_feature (42)..(42): 2′ Fluorine
  misc_feature (44)..(45): 2′ Fluorine
  misc_feature (47)..(47): conjugated to PLK1 via disulfide bond
  misc_feature (47)..(47): 2′ Fluorine

SEQ ID NO: 38 - DNA, 58 bp, Artificial
  Synthetic
  misc_feature (7)..(8): 2′ Fluorine
  misc_feature (11)..(12): 2′ Fluorine
  misc_feature (18)..(18): 2′ Fluorine
  misc_feature (20)..(20): 2′ Fluorine
  misc_feature (23)..(23): 2′ Fluorine
  misc_feature (25)..(26): 2′ Fluorine
  misc_feature (29)..(29): 2′ Fluorine
  misc_feature (33)..(35): 2′ Fluorine
  misc_feature (37)..(37): 2′ Fluorine
  misc_feature (38)..(39): 2′ Fluorine
  misc_feature (38)..(39): 2′ Fluorine
  misc_feature (43)..(44): 2′ Fluorine
  misc_feature (47)..(47): 2′ Fluorine
  misc_feature (49)..(49): 2′ Fluorine
  misc_feature (49)..(49): 2′ Fluorine
  misc_feature (51)..(51): 2′ Fluorine
  misc_feature (58)..(58): 2′ Fluorine
gggaaacuagauuggaacacaguauuggacagucugauuggacugacacauuggagac

SEQ ID NO: 39 - DNA, 58 bp, Artificial; identical to SEQ ID NO: 38
  Synthetic
  misc_feature (7)..(8): 2′ Fluorine
  misc_feature (11)..(12): 2′ Fluorine
  misc_feature (18)..(18): 2′ Fluorine
  misc_feature (20)..(20): 2′ Fluorine
  misc_feature (23)..(23): 2′ Fluorine
  misc_feature (25)..(26): 2′ Fluorine
  misc_feature (29)..(29): 2′ Fluorine
  misc_feature (33)..(35): 2′ Fluorine
  misc_feature (37)..(37): 2′ Fluorine
  misc_feature (38)..(39): 2′ Fluorine
  misc_feature (38)..(39): 2′ Fluorine
  misc_feature (43)..(44): 2′ Fluorine
  misc_feature (47)..(47): 2′ Fluorine
  misc_feature (49)..(49): 2′ Fluorine
  misc_feature (49)..(49): 2′ Fluorine
  misc_feature (51)..(51): 2′ Fluorine
  misc_feature (58)..(58): 5′ Cy3
  misc_feature (58)..(58): 2′ Fluorine

SEQ ID NO: 40 - DNA, 58 bp, Artificial; identical to SEQ ID NO: 38
  Synthetic
  misc_feature (1)..(58): 1-azidododecane clicked onto 2′ position of A at 8 locations
  misc_feature (7)..(8): 2′ Fluorine
  misc_feature (11)..(12): 2′ Fluorine
  misc_feature (18)..(18): 2′ Fluorine
  misc_feature (20)..(20): 2′ Fluorine
  misc_feature (23)..(23): 2′ Fluorine
  misc_feature (25)..(26): 2′ Fluorine
  misc_feature (29)..(29): 2′ Fluorine
  misc_feature (33)..(35): 2′ Fluorine
  misc_feature (37)..(37): 2′ Fluorine
  misc_feature (38)..(39): 2′ Fluorine
  misc_feature (38)..(39): 2′ Fluorine
  misc_feature (43)..(44): 2′ Fluorine
  misc_feature (47)..(47): 2′ Fluorine
  misc_feature (49)..(49): 2′ Fluorine
  misc_feature (49)..(49): 2′ Fluorine
  misc_feature (51)..(51): 2′ Fluorine
  misc_feature (58)..(58): 5′ Cy3
  misc_feature (58)..(58): 2′ Fluorine

SEQ ID NO: 41 - DNA, 58 bp, Artificial; identical to SEQ ID NO: 38
  Synthetic
  misc_feature (1)..(58): phosphorothioate backbone
  misc_feature (7)..(8): 2′ Fluorine
  misc_feature (11)..(12): 2′ Fluorine
  misc_feature (18)..(18): 2′ Fluorine
  misc_feature (20)..(20): 2′ Fluorine
  misc_feature (23)..(23): 2′ Fluorine
  misc_feature (25)..(26): 2′ Fluorine
  misc_feature (29)..(29): 2′ Fluorine
  misc_feature (33)..(35): 2′ Fluorine
  misc_feature (37)..(37): 2′ Fluorine
  misc_feature (38)..(39): 2′ Fluorine
  misc_feature (38)..(39): 2′ Fluorine
  misc_feature (43)..(44): 2′ Fluorine
  misc_feature (47)..(47): 2′ Fluorine
  misc_feature (49)..(49): 2′ Fluorine
  misc_feature (49)..(49): 2′ Fluorine
  misc_feature (51)..(51): 2′ Fluorine
  misc_feature (58)..(58): 5′ Cy3
  misc_feature (58)..(58): 2′ Fluorine

SEQ ID NO: 42 - DNA, 58 bp, Artificial; identical to SEQ ID NO: 38
  Synthetic
  misc_feature (1)..(58): 1-azidododecane clicked onto the 2′ position of A at 4 locations
  misc_feature (7)..(8): 2′ Fluorine
  misc_feature (11)..(12): 2′ Fluorine
  misc_feature (18)..(18): 2′ Fluorine
  misc_feature (20)..(20): 2′ Fluorine
  misc_feature (23)..(23): 2′ Fluorine
  misc_feature (25)..(26): 2′ Fluorine
  misc_feature (29)..(29): 2′ Fluorine
  misc_feature (33)..(35): 2′ Fluorine
  misc_feature (37)..(37): 2′ Fluorine
  misc_feature (38)..(39): 2′ Fluorine
  misc_feature (38)..(39): 2′ Fluorine
  misc_feature (43)..(44): 2′ Fluorine
  misc_feature (47)..(47): 2′ Fluorine
  misc_feature (49)..(49): 2′ Fluorine
  misc_feature (49)..(49): 2′ Fluorine
  misc_feature (51)..(51): 2′ Fluorine
  misc_feature (58)..(58): 5′ Cy3
  misc_feature (58)..(58): 2′ Fluorine

SEQ ID NO: 43 - DNA, 58 bp, Artificial; identical to SEQ ID NO: 38
  Synthetic
  misc_feature (1)..(6): 2′Moe
  misc_feature (7)..(8): 2′ Fluorine
  misc_feature (9)..(11): 2′Moe
  misc_feature (11)..(12): 2′ Fluorine
  misc_feature (14)..(17): 2′Moe
  misc_feature (14)..(24): 2′Moe
  misc_feature (18)..(18): 2′ Fluorine
  misc_feature (19)..(19): 2′Moe
  misc_feature (20)..(20): 2′ Fluorine
  misc_feature (21)..(22): 2′Moe
  misc_feature (23)..(23): 2′ Fluorine
  misc_feature (25)..(26): 2′ Fluorine
  misc_feature (27)..(29): 2′Moe
  misc_feature (29)..(29): 2′ Fluorine
  misc_feature (31)..(32): 2′Moe
  misc_feature (33)..(35): 2′ Fluorine
  misc_feature (36)..(37): 2′Moe
  misc_feature (37)..(37): 2′ Fluorine
  misc_feature (38)..(39): 2′ Fluorine
  misc_feature (38)..(39): 2′ Fluorine
  misc_feature (40)..(42): 2′Moe
  misc_feature (43)..(44): 2′ Fluorine
  misc_feature (45)..(46): 2′Moe
  misc_feature (47)..(47): 2′ Fluorine
  misc_feature (48)..(48): 2′Moe
  misc_feature (49)..(49): 2′ Fluorine
  misc_feature (49)..(49): 2′ Fluorine
  misc_feature (50)..(50): 2′Moe
  misc_feature (51)..(51): 2′ Fluorine
  misc_feature (52)..(57): 2′Moe
  misc_feature (58)..(58): 5′Cy3
  misc_feature (58)..(58): 2′ Fluorine

SEQ ID NO: 44 - DNA, 58 bp, Artificial; identical to SEQ ID NO: 38
  Synthetic
  misc_feature (7)..(8): 2′ Fluorine
  misc_feature (11)..(12): 2′ Fluorine
  misc_feature (18)..(18): 2′ Fluorine
  misc_feature (20)..(20): 2′ Fluorine
  misc_feature (23)..(23): 2′ Fluorine
  misc_feature (25)..(26): 2′ Fluorine
  misc_feature (29)..(29): 2′ Fluorine
  misc_feature (33)..(35): 2′ Fluorine
  misc_feature (37)..(37): 2′ Fluorine
  misc_feature (38)..(39): 2′ Fluorine
  misc_feature (38)..(39): 2′ Fluorine
  misc_feature (43)..(44): 2′ Fluorine
  misc_feature (47)..(47): 2′ Fluorine
  misc_feature (49)..(49): 2′ Fluorine
  misc_feature (49)..(49): 2′ Fluorine
  misc_feature (51)..(51): 2′ Fluorine
  misc_feature (58)..(58): 5′ Cy3
  misc_feature (58)..(58): 2′ Fluorine

SEQ ID NO: 45 - DNA, 58 bp, Artificial; identical to SEQ ID NO: 38
  Synthetic
  misc_feature (1)..(58): PEG-7 clicked onto 2′ position of A at 19 locations
  misc_feature (7)..(8): 2′ Fluorine
  misc_feature (11)..(12): 2′ Fluorine
  misc_feature (18)..(18): 2′ Fluorine
  misc_feature (20)..(20): 2′ Fluorine
  misc_feature (23)..(23): 2′ Fluorine
  misc_feature (25)..(26): 2′ Fluorine
  misc_feature (29)..(29): 2′ Fluorine
  misc_feature (33)..(35): 2′ Fluorine
  misc_feature (37)..(37): 2′ Fluorine
  misc_feature (38)..(39): 2′ Fluorine
  misc_feature (38)..(39): 2′ Fluorine
  misc_feature (43)..(44): 2′ Fluorine
  misc_feature (47)..(47): 2′ Fluorine
  misc_feature (49)..(49): 2′ Fluorine
  misc_feature (49)..(49): 2′ Fluorine
  misc_feature (51)..(51): 2′ Fluorine
  misc_feature (58)..(58): 5′ Cy3
  misc_feature (58)..(58): 2′ Fluorine

SEQ ID NO: 46 - DNA, 58 bp, Artificial; identical to SEQ ID NO: 38
  Synthetic
  misc_feature (1)..(1): 3′ TEG cholesterol
  misc_feature (7)..(8): 2′ Fluorine
  misc_feature (11)..(12): 2′ Fluorine
  misc_feature (18)..(18): 2′ Fluorine
  misc_feature (20)..(20): 2′ Fluorine
  misc_feature (23)..(23): 2′ Fluorine
  misc_feature (25)..(26): 2′ Fluorine
  misc_feature (29)..(29): 2′ Fluorine
  misc_feature (33)..(35): 2′ Fluorine
  misc_feature (37)..(37): 2′ Fluorine
  misc_feature (38)..(39): 2′ Fluorine
  misc_feature (38)..(39): 2′ Fluorine
  misc_feature (43)..(44): 2′ Fluorine
  misc_feature (47)..(47): 2′ Fluorine
  misc_feature (49)..(49): 2′ Fluorine
  misc_feature (49)..(49): 2′ Fluorine
  misc_feature (51)..(51): 2′ Fluorine
  misc_feature (58)..(58): 5′ Cy3
  misc_feature (58)..(58): 2′ Fluorine

SEQ ID NO: 47 - DNA, 58 bp, Artificial; identical to SEQ ID NO: 38
  Synthetic
  misc_feature (1)..(58): 2′ Moe

SEQ ID NO: 48 - DNA, 13 bp, Artificial
  Synthetic
  misc_feature (2)..(4): 2′ Fluorine
  misc_feature (7)..(8): 2′ Fluorine
  misc_feature (12)..(13): 2′ Fluorine
accugaccauguc

SEQ ID NO: 49 - DNA, 28 bp, Artificial
  Synthetic
  misc_feature (2)..(3): 2′ Fluorine
  misc_feature (6)..(7): 2′ Fluorine
  misc_feature (9)..(9): 2′ Fluorine
  misc_feature (12)..(12): 2′ Fluorine
  misc_feature (14)..(15): 2′ Fluorine
  misc_feature (17)..(17): 2′ Fluorine
  misc_feature (19)..(19): 2′ Fluorine
  misc_feature (23)..(24): 2′ Fluorine
  misc_feature (26)..(27): 2′ Fluorine
acugaucguagcacuguacaagucaccg

SEQ ID NO: 50 - DNA, 13 bp, Artificial
  Synthetic
  misc_feature (1)..(1): 2′ Fluorine
  misc_feature (4)..(4): 2′ Fluorine
  misc_feature (7)..(9): 2′ Fluorine
  misc_feature (11)..(11): 2′ Fluorine
  misc_feature (13)..(13): 2′ Fluorine
cggugacuuguac

SEQ ID NO: 51 - DNA, 28 bp, Artificial
  Synthetic
  misc_feature (3)..(3): 2′ Fluorine
  misc_feature (5)..(6): 2′ Fluorine
  misc_feature (8)..(8): 2′ Fluorine
  misc_feature (11)..(12): 2′ Fluorine
  misc_feature (15)..(15): 2′ Fluorine
  misc_feature (18)..(18): 2′ Fluorine
  misc_feature (20)..(20): 2′ Fluorine
  misc_feature (23)..(24): 2′ Fluorine
  misc_feature (28)..(28): 2′ Fluorine
agugcuacgaucagugacauggucaggu

SEQ ID NO: 52 - DNA, 15 bp, Artificial
  Sythetic
  misc_feature (1)..(15): Ppib sense strand
caaauuccaucguga

SEQ ID NO: 53 - DNA, 15 bp, Artificial
  Synthetic
  misc_feature (1)..(3): PTO linkage
  misc_feature (1)..(1): 2′ Fluorine
  misc_feature (2)..(2): 2′ Moe
  misc_feature (3)..(3): 2′ Fluorine
  misc_feature (4)..(4): 2′ Moe
  misc_feature (5)..(5): 2′ Fluorine
  misc_feature (6)..(6): 2′ Moe
  misc_feature (7)..(7): 2′ Fluorine
  misc_feature (8)..(8): 2′ Moe
  misc_feature (9)..(9): 2′ Fluorine
  misc_feature (10)..(10): 2′ Moe
  misc_feature (11)..(11): 2′ Fluorine
  misc_feature (12)..(12): 2′ Moe
  misc_feature (13)..(15): PTO linkage
  misc_feature (13)..(13): 2′ Fluorine
  misc_feature (14)..(14): 2′ Moe
  misc_feature (15)..(15): 2′ Fluorine
caaauuccaucguga

SEQ ID NO: 54 - DNA, 15 bp, Artificial
  Synthetic
  misc_feature (1)..(1): 5′ amine
  misc_feature (1)..(3): PTO linkage
  misc_feature (1)..(1): 2′ Fluorine
  misc_feature (2)..(2): 2′ Moe
  misc_feature (3)..(3): 2′ Fluorine
  misc_feature (4)..(4): 2′ Moe
  misc_feature (5)..(5): 2′ Fluorine
  misc_feature (6)..(6): 2′ Moe
  misc_feature (7)..(7): 2′ Fluorine
  misc_feature (8)..(8): 2′ Moe
  misc_feature (9)..(9): 2′ Fluorine
  misc_feature (10)..(10): 2′ Moe
  misc_feature (11)..(11): 2′ Fluorine
  misc_feature (12)..(12): 2′ Moe
  misc_feature (13)..(15): PTO linkage
  misc_feature (13)..(13): 2′ Fluorine
  misc_feature (14)..(14): 2′ Moe
  misc_feature (15)..(15): 2′ Fluorine
caaauuccaucguga

SEQ ID NO: 55 - DNA, 15 bp, Artificial
  Synthetic
  misc_feature (1)..(1): 5′ tetrazine
  misc_feature (1)..(3): PTO linkage
  misc_feature (1)..(1): 2′ Fluorine
  misc_feature (2)..(2): 2′ Moe
  misc_feature (3)..(3): 2′ Fluorine
  misc_feature (4)..(4): 2′ Moe
  misc_feature (5)..(5): 2′ Fluorine
  misc_feature (6)..(6): 2′ Moe
  misc_feature (7)..(7): 2′ Fluorine
  misc_feature (8)..(8): 2′ Moe
  misc_feature (9)..(9): 2′ Fluorine
  misc_feature (10)..(10): 2′ Moe
  misc_feature (11)..(11): 2′ Fluorine
  misc_feature (12)..(12): 2′ Moe
  misc_feature (13)..(15): PTO linkage
  misc_feature (13)..(13): 2′ Fluorine
  misc_feature (14)..(14): 2′ Moe
  misc_feature (15)..(15): 2′ Fluorine
caaauuccaucguga

SEQ ID NO: 56 - DNA, 15 bp, Artificial
  Synthetic
  misc_feature (1)..(1): 5′ Cy3
  misc_feature (1)..(3): PTO linkage
  misc_feature (1)..(1): 2′ Fluorine
  misc_feature (2)..(2): 2′ Moe
  misc_feature (3)..(3): 2′ Fluorine
  misc_feature (4)..(4): 2′ Moe
  misc_feature (5)..(5): 2′ Fluorine
  misc_feature (6)..(6): 2′ Moe
  misc_feature (7)..(7): 2′ Fluorine
  misc_feature (8)..(8): 2′ Moe
  misc_feature (9)..(9): 2′ Fluorine
  misc_feature (10)..(10): 2′ Moe
  misc_feature (11)..(11): 2′ Fluorine
  misc_feature (12)..(12): 2′ Moe
  misc_feature (13)..(15): PTO linkage
  misc_feature (13)..(13): 2′ Fluorine
  misc_feature (14)..(14): 2′ Moe
  misc_feature (15)..(15): 2′ Fluorine
caaauuccaucguga

SEQ ID NO: 57 - DNA, 20 bp, Artificial
  Synthetic
  misc_feature (1)..(20): Ppib antisense strand
ucacgauggaauuugcuguu

SEQ ID NO: 58 - DNA, 20 bp, Artificial
  Synthetic
  misc_feature (1)..(1): 2′ Moe
  misc_feature (2)..(4): PTO linkage
  misc_feature (2)..(2): 2′ Fluorine
  misc_feature (3)..(3): 2′ Moe
  misc_feature (4)..(4): 2′ Fluorine
  misc_feature (5)..(5): 2′ Moe
  misc_feature (6)..(6): 2′ Fluorine
  misc_feature (7)..(7): 2′ Moe
  misc_feature (8)..(8): 2′ Fluorine
  misc_feature (9)..(9): 2′ Moe
  misc_feature (10)..(10): 2′ Fluorine
  misc_feature (11)..(20): PTO linkage
  misc_feature (11)..(11): 2′ Moe
  misc_feature (12)..(12): 2′ Fluorine
  misc_feature (13)..(13): 2′ Moe
  misc_feature (14)..(14): 2′ Fluorine
  misc_feature (15)..(15): 2′ Moe
  misc_feature (16)..(16): 2′ Fluorine
  misc_feature (17)..(17): 2′ Moe
  misc_feature (18)..(18): 2′ Fluorine
  misc_feature (19)..(19): 2′ Moe
  misc_feature (20)..(20): 2′ Fluorine
ucacgauggaauuugcuguu

SEQ ID NO: 59 - DNA, 21 bp, Artificial
  Synthetic
  misc_feature (2)..(2): 2′ Fluorine
  misc_feature (5)..(6): 2′ Fluorine
  misc_feature (8)..(8): 2′ Fluorine
  misc_feature (10)..(10): 2′ Fluorine
  misc_feature (14)..(14): 2′ Fluorine
  misc_feature (18)..(18): 2′ Fluorine
gcaauuacaugagcgagcatt

SEQ ID NO: 60 - DNA, 21 bp, Artificial
  Synthetic
gcaauuacaugagcgagcatt

SEQ ID NO: 61 - DNA, 21 bp, Artificial
  Synthetic
  misc_feature (1)..(1): 2′ Fluorine
  misc_feature (3)..(5): 2′ Fluorine
  misc_feature (7)..(9): 2′ Fluorine
  misc_feature (11)..(11): 2′ Fluorine
  misc_feature (13)..(13): 2′ Fluorine
  misc_feature (16)..(17): 2′ Fluorine
  misc_feature (19)..(19): 2′ Fluorine
ugcucgcucauguaauugcgg

SEQ ID NO: 62 - DNA, 21 bp, Artificial
  Synthetic
ugcucgcucauguaauugcgg

SEQ ID NO: 63 - protein, 23 aa, Artificial
  Synthetic
GLFGAIAGFIENGWEGMIDGWYG

SEQ ID NO: 64 - protein, 14 aa, Artificial
  Synthetic
LAEALAEALEALAA

SEQ ID NO: 65 - protein, 4 aa, Artificial
  Synthetic
HHHH

SEQ ID NO: 66 - protein, 8 aa, Artificial
  Synthetic
HHHHHHHH

SEQ ID NO: 67 - protein, 12 aa, Artificial
  Synthetic
HHHHHHHHHHHH

SEQ ID NO: 68 - DNA, 20 bp, Artificial
  Synthetic
gtttggcaaagttctagagg

SEQ ID NO: 69 - DNA, 20 bp, Artificial
  Synthetic
acatccttcaggggtttatc

SEQ ID NO: 70 - DNA, 18 bp, Artificial
  Synthetic
cttcgctggctcccactt

SEQ ID NO: 71 - DNA, 23 bp, Artificial
  Synthetic
ccattgaaatcctgagtgatgtg

SEQ ID NO: 72 - DNA, 27 bp, Artificial
  Synthetic
cttgtcggccatgatatagacgttgtg

SEQ ID NO: 73 - DNA, 27 bp, Artificial sequence
  Synthetic
cuugucggccaugauauagacguugug

SEQ ID NO: 74 - DNA, 34 bp, Artificial Sequence
  Synthetic
  misc_feature (3)..(3): 2′ Ome
  misc_feature (7)..(7): 2′ Ome
  misc_feature (11)..(11): 2′ Ome
  misc_feature (15)..(15): 2′ Ome
  misc_feature (19)..(19): 2′ Ome
  misc_feature (24)..(24): 2′ Ome
  misc_feature (28)..(28): 2′ Ome
gcutctcgtcacgaactccagcaggautccuact

SEQ ID NO: 75 - DNA, 34 bp, Artificial Sequence
  Synthetic
  misc_feature (2)..(2): 2′ OMe
  misc_feature (6)..(6): 2′ OMe
  misc_feature (12)..(12): 2′ Ome
  misc_feature (16)..(16): 2′ Ome
  misc_feature (20)..(20): 2′ Ome
  misc_feature (24)..(24): 2′ OMe
  misc_feature (30)..(30): 2′ Ome
  misc_feature (34)..(34): 2′ OMe
gggtgctcactcgtccatgccgagagtacaggtg

SEQ ID NO: 76 - DNA, 43 bp, Artificial Sequence
  Synthetic
  misc_feature (1)..(1): 2′ OMe
  misc_feature (5)..(5): 2′ OMe
  misc_feature (9)..(9): 2′ OMe
  misc_feature (13)..(13): 2′ OMe
  misc_feature (17)..(17): 2′ OMe
  misc_feature (21)..(21): 2′ OMe
  misc_feature (25)..(21): 2′ OMe
  misc_feature (33)..(33): 2′ OMe
  misc_feature (37)..(37): 2′ OMe
  misc_feature (41)..(41): 2′ OMe
cggggccgugcagcttaattaagcggagaaggcaagccccgca

SEQ ID NO: 77 - DNA, 32 bp, Artificial Sequence
  Synthetic
  misc_feature (2)..(2): 2′ OMe
  misc_feature (6)..(6): 2′ OMe
  misc_feature (10)..(10): 2′ OMe
  misc_feature (14)..(14): 2′ OMe
  misc_feature (18)..(18): 2′ OMe
  misc_feature (22)..(22): 2′ OMe
  misc_feature (26)..(26): 2′ OMe
  misc_feature (30)..(30): 2′ OMe
gcggcggttggggucttugctcagcgatgutg

SEQ ID NO: 78 - DNA, 32 bp, Artificial Sequence
  Synthetic
  misc_feature (3)..(3): 2′ OMe
  misc_feature (7)..(7): 2′ OMe
  misc_feature (9)..(9): 2′ OMe
  misc_feature (13)..(13): 2′ OMe
  misc_feature (17)..(17): 2′ OMe
  misc_feature (21)..(21): 2′ OMe
  misc_feature (27)..(27): 2′ OMe
  misc_feature (31)..(31): 2′ OMe
ttgtacagggtagtggutgtcggggguagtgg

SEQ ID NO: 79 - DNA, 33 bp, Artificial Sequence
  Synthetic
  misc_feature (1)..(1): 2′ OMe
  misc_feature (5)..(5): 2′ OMe
  misc_feature (9)..(9): 2′ OMe
  misc_feature (13)..(13): 2′ OMe
  misc_feature (17)..(17): 2′ OMe
  misc_feature (21)..(21): 2′ OMe
  misc_feature (25)..(25): 2′ OMe
  misc_feature (27)..(27): 2′ OMe
  misc_feature (31)..(31): 2′ OMe
caggctttattcaaagaccaagaggtgatcccg

SEQ ID NO: 80 - DNA, 33 bp, Artificial Sequence
  Synthetic
  misc_feature (4)..(4): 2′ Ome
  misc_feature (8)..(8): 2′ Ome
  misc_feature (12)..(12): 2′ Ome
  misc_feature (16)..(16): 2′ Ome
  misc_feature (20)..(20): 2′ Ome
  misc_feature (24)..(24): 2′ Ome
  misc_feature (28)..(28): 2′ Ome
  misc_feature (32)..(32): 2′ Ome
caagggagagaagaagggcatggccccgcttac

SEQ ID NO: 81 - DNA, 989 bp, Artificial Sequence
  Synthetic
aagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 82 - DNA, 33 bp, Artificial Sequence
  Synthetic
tttcttcttactcttcttttctctcggtcagct

SEQ ID NO: 83 - DNA, 33 bp, Artificial Sequence
  Synthetic
tcctcgcccttgctcaccatggtggctcgccgg

SEQ ID NO: 84 - DNA, 25 bp, Artificial
  Synthetic
gatgggcaccaccccgggtcgccgt

SEQ ID NO: 85 - DNA, 34 bp, Artificial
  Synthetic
tgccgtaggggtggtgcagatgaactcttgaaga

SEQ ID NO: 86 - DNA, 34 bp, Artificial
  Synthetic
acacgctgacacgagggtgggccaggttcgggca

SEQ ID NO: 87 - DNA, 34 bp, Artificial
  Synthetic
ccagctcgagctgaagcactgcacgccttcatgt

SEQ ID NO: 88 - DNA, 32 bp, Artificial
  Synthetic
agcttgcctggcatcgccctcgccctcttata

SEQ ID NO: 89 - DNA, 32 bp, Artificial
  Synthetic
agggtggtacttgtggccgtttactgaacagc

SEQ ID NO: 90 - DNA, 34 bp, Artificial
  Synthetic
agatggtgcggtcttgtagttgccgtgttcttct

SEQ ID NO: 91 - DNA, 34 bp, Artificial
  Synthetic
tggcggactcagggtgtcgccctcgagtggctgt

SEQ ID NO: 92 - DNA, 34 bp, Artificial
  Synthetic
ggtcggggtgaagtcgatgcccttcacaggatgt

SEQ ID NO: 93 - DNA, 32 bp, Artificial
  Synthetic
tcggcgcggctcctggacgtagccgcacgggc

SEQ ID NO: 94 - DNA, 32 bp, Artificial
  Synthetic
cggttcactgaagaagtcgtgctgcgtaggtc

SEQ ID NO: 95 - DNA, 34 bp, Artificial
  Synthetic
gcttgtcggcttgaagttcaccttgagtgatcgc

SEQ ID NO: 96 - DNA, 34 bp, Artificial
  Synthetic
tgtagttgtctgcacgctgccgtcctggcggact

SEQ ID NO: 97 - DNA, 34 bp, Artificial
  Synthetic
tgccgtcctgatgggggtgttctgctcagcagca

SEQ ID NO: 98 - DNA, 32 bp, Artificial
  Synthetic
tggcggatccatgatatagacgttacttcacc

SEQ ID NO: 99 - DNA, 32 bp, Artificial
  Synthetic
tcggcgagactccagcttgtgcccgctcgatg

SEQ ID NO: 100 - DNA, 34 bp, Artificial
  Synthetic
gcttctcgtcacgaactccagcaggattcctact

SEQ ID NO: 101 - DNA, 34 bp, Artificial; identical to SEQ ID NO: 75
  Synthetic

SEQ ID NO: 102 - DNA, 43 bp, Artificial
  Synthetic
cggggccgtgcagcttaattaagcggagaaggcaagccccgca

SEQ ID NO: 103 - DNA, 32 bp, Artificial
  Synthetic
gcggcggttggggtctttgctcagcgatgttg

SEQ ID NO: 104 - DNA, 32 bp, Artificial
  Synthetic
ttgtacagggtagtggttgtcgggggtagtgg

SEQ ID NO: 105 - DNA, 33 bp, Artificial; identical to SEQ ID NO: 79
  Synthetic

SEQ ID NO: 106 - DNA, 33 bp, Artificial; identical to SEQ ID NO: 80
  Synthetic

SEQ ID NO: 107 - DNA, 42 bp, Artificial
  Synthetic
tcgtgctgcttcattctctctcttcctactcaggcggggtgt

SEQ ID NO: 108 - DNA, 42 bp, Artificial
  Synthetic
gtggtcgggcatggcggacttgctgcactctgctggtagtgg

SEQ ID NO: 109 - DNA, 42 bp, Artificial
  Synthetic
gctgccgtcctcgagccttcggggtagcggctgaatatttct

SEQ ID NO: 110 - DNA, 42 bp, Artificial
  Synthetic
tgttgtgggccgtcgccgatgtttattctcttactcttcttt

SEQ ID NO: 111 - DNA, 42 bp, Artificial
  Synthetic
aaagaccaagaggtagcacgggcggatcttgaagtgatggtg

SEQ ID NO: 112 - DNA, 42 bp, Artificial
  Synthetic
acgccgtaggtcagtgctcaccaagggagagaagaaggtagt

SEQ ID NO: 113 - DNA, 42 bp, Artificial
  Synthetic
acaggtgcatggtggctcttagcactgccgctcctggacgta

SEQ ID NO: 114 - DNA, 42 bp, Artificial
  Synthetic
ggtggtctcgtccttgaagaatcaccttggttgtcgggcagc

SEQ ID NO: 115 - DNA, 42 bp, Artificial
  Synthetic
gatgccgttcttctgttgccgacgagggtgggccacggtgaa

SEQ ID NO: 116 - DNA, 42 bp, Artificial
  Synthetic
gcttgtccggactgggtgctcagggcatcagctcctcgccct

SEQ ID NO: 117 - DNA, 42 bp, Artificial
  Synthetic
ggccagaaggcaagctcagggggccatgatatagacacctcg

SEQ ID NO: 118 - DNA, 42 bp, Artificial
  Synthetic
ggcagcttgccggtcgaccaggaaggcagcttaatcgcttct

SEQ ID NO: 119 - DNA, 42 bp, Artificial
  Synthetic
ccccgcagatgggcaccacccgggcacggcgcgggtcttgta

SEQ ID NO: 120 - DNA, 42 bp, Artificial
  Synthetic
ggtgcagtcgccctcgaacttcgttgtgcgttggggtctttg

SEQ ID NO: 121 - DNA, 42 bp, Artificial
  Synthetic
gctgttgtagttgtcagggtgatgaacttcagggtcgtttac

SEQ ID NO: 122 - DNA, 42 bp, Artificial
  Synthetic
actccagggaccatgtgatcgtaagcgggtcgccgtccagct

SEQ ID NO: 123 - DNA, 28 bp, Artificial
  Synthetic
ccgcttacttgtactccagcacttgtgc

SEQ ID NO: 124 - DNA, 35 bp, Artificial
  Synthetic
cccaggacttcagcccgtaggtggcatcgccctcg

SEQ ID NO: 125 - DNA, 42 bp, Artificial
  Synthetic
agctcgtgctgaacttgtggccagcttgtcgatgcggttcac

SEQ ID NO: 126 - DNA, 28 bp, Artificial
  Synthetic
ccctcgccggacacccatgccgagagtg

SEQ ID NO: 127 - DNA, 35 bp, Artificial
  Synthetic
ttgaagtcgatgcctgttgcccggcggtcacgaac

SEQ ID NO: 128 - DNA, 42 bp, Artificial
  Synthetic
ggtcacgaactccatttttttctctcttggtcagcatgaact

SEQ ID NO: 129 - DNA, 28 bp, Artificial
  Synthetic
gaccatggttcacctgccgttagttgcc

SEQ ID NO: 130 - DNA, 35 bp, Artificial
  Synthetic
ggtgcgcggcgcgggtcttgtcttctgcttctttt

SEQ ID NO: 131 - DNA, 28 bp, Artificial
  Synthetic
ttgtcggtggcggatcttgaatgatcgc

SEQ ID NO: 132 - DNA, 42 bp, Artificial
  Synthetic
tccttgaagaagatggtgcagttgccgtaggtggccttactc

SEQ ID NO: 133 - DNA, 35 bp, Artificial
  Synthetic
tttattcatttcttatcgcccggcagcttgccggt

SEQ ID NO: 134 - DNA, 35 bp, Artificial
  Synthetic
cttcctactcaggccggcggcgcttctcgttgggg

SEQ ID NO: 135 - DNA, 35 bp, Artificial
  Synthetic
acgctgccgtcctctctttgcgccgagagtgatcc

SEQ ID NO: 136 - DNA, 35 bp, Artificial
  Synthetic
gatgttgccatgattcacctctcctggacgtagcc

SEQ ID NO: 137 - DNA, 42 bp, Artificial
  Synthetic
acgagggtgggccattcgggcgtcgccctcgaactatagacg

SEQ ID NO: 138 - DNA, 35 bp, Artificial
  Synthetic
gggcacgtcgccctcgccggaatggtggttgtggc

SEQ ID NO: 139 - DNA, 28 bp, Artificial
  Synthetic
ctcttataaagaccaagaggtcgtccat

SEQ ID NO: 140 - DNA, 35 bp, Artificial
  Synthetic
ttacttgtacagctacaggtggctcacccacgctg

SEQ ID NO: 141 - DNA, 35 bp, Artificial
  Synthetic
tcagggcggactgggagctgctgttgtaccagggt

SEQ ID NO: 142 - DNA, 35 bp, Artificial
  Synthetic
gaagtcgctcgatgcggttcagttgtaccgccctt

SEQ ID NO: 143 - DNA, 28 bp, Artificial
  Synthetic
tccagcttggtagtggtcggcgtgctca

SEQ ID NO: 144 - DNA, 42 bp, Artificial
  Synthetic
atggcggacttgaaggtggtcaacttgtggccgttagctcct

SEQ ID NO: 145 - DNA, 28 bp, Artificial
  Synthetic
ggtgaactacgtcgacgccgtaggtcag

SEQ ID NO: 146 - DNA, 42 bp, Artificial
  Synthetic
caagggagagaagacggccgcggtagtggttgtcggttctgc

SEQ ID NO: 147 - DNA, 35 bp, Artificial
  Synthetic
tgggggtggcagcagcagcttaattaagagggcat

SEQ ID NO: 148 - DNA, 35 bp, Artificial
  Synthetic
tgtgcccccttcagtgctgcttcatgtggcactgc

SEQ ID NO: 149 - DNA, 35 bp, Artificial
  Synthetic
ggctgaagtcggggcttgaagtcgatgccaggatg

SEQ ID NO: 150 - DNA, 35 bp, Artificial
  Synthetic
ccgtccagctcgacatgggcattgccgttcgccga

SEQ ID NO: 151 - DNA, 35 bp, Artificial
  Synthetic
ccaccccggccagaaggcaaggcagaaggcacggg

SEQ ID NO: 152 - DNA, 40 bp, Artificial
  Synthetic
ttctctcttctctcttgagatctcgccactctagtccgga

SEQ ID NO: 153 - DNA, 24 bp, Artificial
  Synthetic
tccggactagagtggcgagatctc

SEQ ID NO: 154 - DNA, 37 bp, Artificial
  Synthetic
cgttgtggctgttgtagttgtactcttcttactcttc

SEQ ID NO: 155 - DNA, 25 bp, Artificial
  Synthetic
tgccgttcttctgcttgtcggccat

SEQ ID NO: 156 - DNA, 64 bp, Artificial
  Synthetic
tcgtgctgcttcatgtggtcggggtagcggcttagtggtcggcgagctgcacgctgccgtcctc

SEQ ID NO: 157 - DNA, 64 bp, Artificial
  Synthetic
ttgtacagctcgtccatgccgagagtgatcccgatgaacttcagggtcagcttgccgtaggtgg

SEQ ID NO: 158 - DNA, 31 bp, Artificial
  Synthetic
caggatgggcaccacctacaggtgcaaggga

SEQ ID NO: 159 - DNA, 31 bp, Artificial
  Synthetic
acgttgtggctgttgtagttgccgtcgtcct

SEQ ID NO: 160 - DNA, 21 bp, Artificial Sequence
  Synthetic
  misc_feature (21)..(21): Branching unit
gcaauuacaugagcgagcatt

SEQ ID NO: 161 - DNA, 47 bp, Artificial Sequence; identical to SEQ ID NO: 7
  Synthetic
  misc_feature (1)..(1): Branching unit

SEQ ID NO: 162 - DNA, 15 bp, Artificial Sequence
  Synthetic
  misc_feature (1)..(1): 2′ Fluorine
  misc_feature (2)..(2): 2′ OMe
  misc_feature (3)..(3): 2′ Fluorine
  misc_feature (4)..(4): 2′ OMe
  misc_feature (5)..(5): 2′ Fluorine
  misc_feature (6)..(6): 2′ OMe
  misc_feature (7)..(7): 2′ Fluorine
  misc_feature (8)..(8): 2′ OMe
  misc_feature (9)..(9): 2′ Fluorine
  misc_feature (10)..(10): 2′ OMe
  misc_feature (11)..(11): 2′ Fluorine
  misc_feature (12)..(12): 2′ OMe
  misc_feature (13)..(13): 2′ Fluorine
  misc_feature (14)..(14): 2′ OMe
  misc_feature (15)..(15): 2′ Fluorine
  misc_feature (15)..(15): Branching unit
caaauuccaucguga

SEQ ID NO: 163 - DNA, 47 bp, Artificial Sequence; identical to SEQ ID NO: 7
  Synthetic
  misc_feature (1)..(1): Branching unit

SEQ ID NO: 164 - DNA, 34 bp, Artificial Sequence
  Synthetic
  misc_feature (1)..(1): 5′ Amine
  misc_feature (2)..(2): 2′ OMe
  misc_feature (4)..(4): 2′ OMe
  misc_feature (6)..(6): 2′ OMe
  misc_feature (8)..(8): 2′ OMe
  misc_feature (10)..(10): 2′ OMe
  misc_feature (12)..(12): 2′ OMe
  misc_feature (14)..(14): 2′ OMe
  misc_feature (21)..(21): 2′ OMe
  misc_feature (23)..(23): 2′ OMe
  misc_feature (25)..(25): 2′ OMe
  misc_feature (27)..(27): 2′ OMe
  misc_feature (29)..(29): 2′ OMe
  misc_feature (31)..(31): 2′ OMe
  misc_feature (33)..(33): 2′ OMe
caaauuccaucgugattttcaaauuccaucguga

SEQ ID NO: 165 - DNA, 15 bp, Artificial Sequence
  Synthetic
  misc_feature (1)..(1): 5′ Cy3
  misc_feature (2)..(2): 2′ OMe
  misc_feature (4)..(4): 2′ OMe
  misc_feature (6)..(6): 2′ OMe
  misc_feature (8)..(8): 2′ OMe
  misc_feature (10)..(10): 2′ OMe
  misc_feature (12)..(12): 2′ OMe
  misc_feature (14)..(14): 2′ OMe
  misc_feature (14)..(14): Branching unit
caaauuccaucguga

SEQ ID NO: 166 - DNA, 15 bp, Artificial Sequence
  Synthetic
  misc_feature (1)..(1): Branching unit
  misc_feature (2)..(2): 2′ OMe
  misc_feature (4)..(4): 2′ OMe
  misc_feature (6)..(6): 2′ OMe
  misc_feature (8)..(8): 2′ OMe
  misc_feature (10)..(10): 2′ OMe
  misc_feature (12)..(12): 2′ OMe
  misc_feature (14)..(14): 2′ OMe
  misc_feature (15)..(15): Cy3
agugcuaccuuaaac